SEQUENCE LISTING (35 sequences)

SEQ ID NO: 1 - protein, 409 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQT

SEQ ID NO: 2 - DNA, 1233 bp, Homo sapiens
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagttcgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagcccaagagtgttcgctggatgatgacaccattctaatcccaattatagttggtgctggtctttcaggcttgattatcgttatagtgattgcttacgtaattggcagaagaaaaagttatgctggatatcagactctgtaa

SEQ ID NO: 3 - DNA, 1233 bp, Artificial Sequence
  variant of LAMP-2
atggtctgcttcagactgttccctgtccctggatctggtctggtgcttgtgtgcttggtgctgggtgctgtgagatcctatgcccttgagctgaacctgactgactcagaaaatgccacttgcctgtatgccaagtggcagatgaacttcactgtgagatatgagactaccaacaagacctacaagactgtgaccatctcagaccatggcactgtcacctacaatggatcaatctgtggtgatgatcagaatggcccaaagatagcagtgcagtttgggcccggtttttcctggattgctaacttcaccaaggcagcctccacctacagcattgactcagtcagcttcagctacaacactggggataacaccaccttccctgacgcagaggacaagggaatccttactgtggacgaactcctggcaatcagaatcccccttaacgacctgttcagatgcaactccctttcaacccttgaaaagaatgatgtggtgcaacactattgggacgtcctggtgcaagcctttgtgcagaatgggacagtgagtaccaacgagttcctctgtgacaaggacaagaccagcactgtggcccccactatccacaccactgtgcccagccctaccactacccccacccctaaagagaagccagaagctggaacctactcagtcaacaatggaaatgacacatgcctccttgccaccatgggactgcagctgaacatcactcaggacaaggtggcctcagtgattaacatcaaccctaacaccactcatagcactgggagctgcagatcacatacagctctgctgaggctcaactcctccaccatcaagtacctggactttgtgtttgctgtgaagaatgagaacaggttctacctcaaggaagtgaacatttccatgtacctggtcaatggttcagtgttctctattgccaacaacaatctgagctactgggatgcacccctgggatcctcctacatgtgcaacaaggagcagactgtgagtgtgtcaggtgcttttcagatcaacacttttgacctgagggtgcagcccttcaatgtgactcagggaaagtactccactgcacaagagtgttccttggatgatgacactatcctcatccccattattgtgggagctggactgtcaggattgattatagtgattgtgattgcttatgtgattggaaggagaaagagctatgctggctaccagaccctgtaa

SEQ ID NO: 4 - DNA, 1233 bp, Artificial Sequence
  variant of LAMP-2
atggtgtgctttagactgtttcctgtgcctggttcagggctggtcctggtctgtctggtgctgggggctgtcagaagctatgccttggagctgaacctcactgatagtgaaaatgccacttgtctgtatgctaagtggcagatgaacttcactgtgagatatgaaaccaccaacaagacttacaaaacagtgaccatctcagatcatggaactgtgacctacaacggcagcatttgtggagacgaccagaacggaccaaaaatcgctgtccaatttgggcctggattctcctggattgccaatttcactaaagctgcctccacatattcaattgactcagtgtccttctcctacaacactggggacaacactactttccctgatgctgaagataagggaatcttgacagtggatgagctgctggctatcaggatccctttgaatgacctgtttaggtgtaattcactgagcactctggagaagaacgacgtggtgcagcactactgggacgtgctggtgcaggcctttgtgcagaacggcactgtgtccaccaacgaattcctgtgtgataaggacaaaacttccactgtggcacctacaattcacactactgtgccttcacctaccaccactccaactccaaaggaaaagcctgaagcaggaacctactctgtgaacaatggcaatgatacctgtctgttggccaccatgggcctccaactgaacattactcaggacaaggtggcctcagtgattaacattaaccccaacactacccactccactggcagctgtagatcacacacagccttgctcagactgaatagcagcaccatcaagtatttggattttgtgtttgcagtgaagaatgaaaacaggttctacctgaaggaagtcaacatctcaatgtacctggtgaacggctcagtgttcagcattgccaacaacaacctctcctattgggacgctccactggggagcagctacatgtgtaacaaggaacagactgtgtcagtgtcaggagccttccagattaacacctttgatctgagggtccaaccctttaatgtcactcaaggaaagtatagcactgcccaggagtgctccctggatgatgacaccattctgattccaatcattgtgggtgcaggactttctgggcttattattgtgattgtgattgcctatgtgattggcagaaggaaatcctatgcagggtaccaaactctgtaa

SEQ ID NO: 5 - DNA, 1233 bp, Artificial Sequence
  variant of LAMP-2
atggtctgttttaggctgttccctgtccctggttcaggactggtcttagtgtgtctggtgcttggagctgtcagaagctatgccctggagctgaacctgactgactcagaaaatgccacttgcctgtatgccaagtggcagatgaacttcactgtcagatatgaaaccaccaacaagacctataagactgtgaccatctcagaccatggcactgtgacttacaatgggtcaatttgtggagatgaccagaatggccctaagatagctgtccagtttggtccaggattcagctggattgccaacttcaccaaggcagccagcacctacagcattgactctgtgtccttctcctacaacacaggagacaacaccactttccctgatgcagaggacaaaggtatcctgactgtggatgagttgctggcaatcaggatcccactgaacgatctgttcaggtgcaactcactgtccactctggaaaagaatgatgtggtgcagcactattgggatgtgctagtccaggcctttgtccagaatgggactgtgtcaactaatgagttcctgtgtgacaaggacaagacaagcactgtagcccccactatccataccacagtacctagccccaccactactccaacccccaaggagaagcctgaggctggcacctactcagtgaacaatgggaatgacacctgtttgctggccactatgggactccaactgaacatcacccaggacaaagtggcctctgtgatcaatatcaatcccaacaccacccacagcactgggtcctgcagaagccacactgccctcctgaggctcaactcatcaactatcaagtacttggattttgtgtttgcagtgaagaatgagaacagattctacctcaaagaggtcaacatttcaatgtacctggtgaatgggagtgtgttctccattgctaacaacaacctgagctactgggatgcccctctgggctcctcatacatgtgcaacaaggaacagactgtgagtgtgtcaggggccttccagatcaacacttttgacctgagagtgcagccctttaatgtgacacagggaaagtacagcactgctcaggagtgcagcctggatgatgacactatcctgatccctatcattgtgggggcaggcctgtctggactcattattgtgattgtgattgcctatgtgatagggagaaggaagtcttatgctggataccagaccctgtaa

SEQ ID NO: 6 - DNA, 13 bp, Unknown
  Kozak sequence
gccgccaccatgg

SEQ ID NO: 7 - DNA, 387 bp, Unknown
  full-length polyA sequence
tggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 8 - DNA, 4549 bp, Artificial Sequence
  expression cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagcggccgccagccgccaccatggtctgcttcagactgttccctgtccctggatctggtctggtgcttgtgtgcttggtgctgggtgctgtgagatcctatgcccttgagctgaacctgactgactcagaaaatgccacttgcctgtatgccaagtggcagatgaacttcactgtgagatatgagactaccaacaagacctacaagactgtgaccatctcagaccatggcactgtcacctacaatggatcaatctgtggtgatgatcagaatggcccaaagatagcagtgcagtttgggcccggtttttcctggattgctaacttcaccaaggcagcctccacctacagcattgactcagtcagcttcagctacaacactggggataacaccaccttccctgacgcagaggacaagggaatccttactgtggacgaactcctggcaatcagaatcccccttaacgacctgttcagatgcaactccctttcaacccttgaaaagaatgatgtggtgcaacactattgggacgtcctggtgcaagcctttgtgcagaatgggacagtgagtaccaacgagttcctctgtgacaaggacaagaccagcactgtggcccccactatccacaccactgtgcccagccctaccactacccccacccctaaagagaagccagaagctggaacctactcagtcaacaatggaaatgacacatgcctccttgccaccatgggactgcagctgaacatcactcaggacaaggtggcctcagtgattaacatcaaccctaacaccactcatagcactgggagctgcagatcacatacagctctgctgaggctcaactcctccaccatcaagtacctggactttgtgtttgctgtgaagaatgagaacaggttctacctcaaggaagtgaacatttccatgtacctggtcaatggttcagtgttctctattgccaacaacaatctgagctactgggatgcacccctgggatcctcctacatgtgcaacaaggagcagactgtgagtgtgtcaggtgcttttcagatcaacacttttgacctgagggtgcagcccttcaatgtgactcagggaaagtactccactgcacaagagtgttccttggatgatgacactatcctcatccccattattgtgggagctggactgtcaggattgattatagtgattgtgattgcttatgtgattggaaggagaaagagctatgctggctaccagaccctgtaaaagggcgaattccagcacacgcgtcctaggagctcgagtactactggcggccgttactagtggatccgcggtacaagtaagcatgcaagcttcgaggacggggtgaactacgcctgaatcaagcttatcgataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctcgataccgtcgatatcgatcctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagcaattcgttgatctgaatttcgaccacccataatagatctcccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - DNA, 4549 bp, Artificial Sequence
  expression cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagcggccgccagccgccaccatggtgtgctttagactgtttcctgtgcctggttcagggctggtcctggtctgtctggtgctgggggctgtcagaagctatgccttggagctgaacctcactgatagtgaaaatgccacttgtctgtatgctaagtggcagatgaacttcactgtgagatatgaaaccaccaacaagacttacaaaacagtgaccatctcagatcatggaactgtgacctacaacggcagcatttgtggagacgaccagaacggaccaaaaatcgctgtccaatttgggcctggattctcctggattgccaatttcactaaagctgcctccacatattcaattgactcagtgtccttctcctacaacactggggacaacactactttccctgatgctgaagataagggaatcttgacagtggatgagctgctggctatcaggatccctttgaatgacctgtttaggtgtaattcactgagcactctggagaagaacgacgtggtgcagcactactgggacgtgctggtgcaggcctttgtgcagaacggcactgtgtccaccaacgaattcctgtgtgataaggacaaaacttccactgtggcacctacaattcacactactgtgccttcacctaccaccactccaactccaaaggaaaagcctgaagcaggaacctactctgtgaacaatggcaatgatacctgtctgttggccaccatgggcctccaactgaacattactcaggacaaggtggcctcagtgattaacattaaccccaacactacccactccactggcagctgtagatcacacacagccttgctcagactgaatagcagcaccatcaagtatttggattttgtgtttgcagtgaagaatgaaaacaggttctacctgaaggaagtcaacatctcaatgtacctggtgaacggctcagtgttcagcattgccaacaacaacctctcctattgggacgctccactggggagcagctacatgtgtaacaaggaacagactgtgtcagtgtcaggagccttccagattaacacctttgatctgagggtccaaccctttaatgtcactcaaggaaagtatagcactgcccaggagtgctccctggatgatgacaccattctgattccaatcattgtgggtgcaggactttctgggcttattattgtgattgtgattgcctatgtgattggcagaaggaaatcctatgcagggtaccaaactctgtaaaagggcgaattccagcacacgcgtcctaggagctcgagtactactggcggccgttactagtggatccgcggtacaagtaagcatgcaagcttcgaggacggggtgaactacgcctgaatcaagcttatcgataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctcgataccgtcgatatcgatcctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagcaattcgttgatctgaatttcgaccacccataatagatctcccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 4549 bp, Artificial Sequence
  expression cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagcggccgccagccgccaccatggtctgttttaggctgttccctgtccctggttcaggactggtcttagtgtgtctggtgcttggagctgtcagaagctatgccctggagctgaacctgactgactcagaaaatgccacttgcctgtatgccaagtggcagatgaacttcactgtcagatatgaaaccaccaacaagacctataagactgtgaccatctcagaccatggcactgtgacttacaatgggtcaatttgtggagatgaccagaatggccctaagatagctgtccagtttggtccaggattcagctggattgccaacttcaccaaggcagccagcacctacagcattgactctgtgtccttctcctacaacacaggagacaacaccactttccctgatgcagaggacaaaggtatcctgactgtggatgagttgctggcaatcaggatcccactgaacgatctgttcaggtgcaactcactgtccactctggaaaagaatgatgtggtgcagcactattgggatgtgctagtccaggcctttgtccagaatgggactgtgtcaactaatgagttcctgtgtgacaaggacaagacaagcactgtagcccccactatccataccacagtacctagccccaccactactccaacccccaaggagaagcctgaggctggcacctactcagtgaacaatgggaatgacacctgtttgctggccactatgggactccaactgaacatcacccaggacaaagtggcctctgtgatcaatatcaatcccaacaccacccacagcactgggtcctgcagaagccacactgccctcctgaggctcaactcatcaactatcaagtacttggattttgtgtttgcagtgaagaatgagaacagattctacctcaaagaggtcaacatttcaatgtacctggtgaatgggagtgtgttctccattgctaacaacaacctgagctactgggatgcccctctgggctcctcatacatgtgcaacaaggaacagactgtgagtgtgtcaggggccttccagatcaacacttttgacctgagagtgcagccctttaatgtgacacagggaaagtacagcactgctcaggagtgcagcctggatgatgacactatcctgatccctatcattgtgggggcaggcctgtctggactcattattgtgattgtgattgcctatgtgatagggagaaggaagtcttatgctggataccagaccctgtaaaagggcgaattccagcacacgcgtcctaggagctcgagtactactggcggccgttactagtggatccgcggtacaagtaagcatgcaagcttcgaggacggggtgaactacgcctgaatcaagcttatcgataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctcgataccgtcgatatcgatcctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagcaattcgttgatctgaatttcgaccacccataatagatctcccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 130 bp, Unknown
  inverted terminal repeat (ITR) sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 130 bp, Unknown
  inverted terminal repeat (ITR) sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - skipped

SEQ ID NO: 14 - RNA, 13 nt, Unknown
  alternative Kozak sequence
  misc_feature (1)..(3): May be absent
  misc_feature (1)..(1): g is the most common base, but may vary
  misc_feature (2)..(3): c is the most common base, but may vary
  misc_feature (4)..(4): g is the most common base, but may vary
  misc_feature (5)..(6): c is the most common base, but may vary
  misc_feature (8)..(9): c is the most common base, but may vary
nnnnnnrnnaugg

SEQ ID NO: 15 - RNA, 8 nt, Unknown
  alternative Kozak sequence
  misc_feature (3)..(4): n is a, c, g, or u
  misc_feature (8)..(8): n is a, c, g, or u
agnnaugn

SEQ ID NO: 16 - RNA, 7 nt, Unknown
  alternative Kozak sequence
  misc_feature (2)..(3): n is a, c, g, or u
annaugg

SEQ ID NO: 17 - RNA, 7 nt, Unknown
  alternative Kozak sequence
accaugg

SEQ ID NO: 18 - RNA, 10 nt, Unknown
  alternative Kozak sequence
gacaccaugg

SEQ ID NO: 19 - DNA, 235 bp, Bos taurus
tcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaggacaatagcaggcatgctggggatgcggtgggctctatggcttctg

SEQ ID NO: 20 - DNA, 222 bp, Simian virus 40
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 21 - DNA, 202 bp, Homo sapiens
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 22 - DNA, 1730 bp, Unknown
  CAG promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 23 - DNA, 204 bp, Human cytomegalovirus
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 24 - DNA, 317 bp, Simian virus 40
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 25 - DNA, 500 bp, Unknown
  PGK promoter
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 26 - DNA, 2403 bp, Homo sapiens
cctgcagggcccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctattctcgcagctcacc

SEQ ID NO: 27 - DNA, 596 bp, Woodchuck hepatitis virus
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 28 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - DNA, 1233 bp, Homo sapiens
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctcaagactgcagtgcagatgacgacaacttccttgtgcccatagcggtgggagctgccttggcaggagtacttattctagtgttgctggcttattttattggtctcaagcaccatcatgctggatatgagcaattttag

SEQ ID NO: 30 - DNA, 1236 bp, Homo sapiens
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaa

SEQ ID NO: 31 - DNA, 1080 bp, Artificial Sequence
  variant of LAMP-2
atggtctgcttcagactgttccctgtccctggatctggtctggtgcttgtgtgcttggtgctgggtgctgtgagatcctatgcccttgagctgaacctgactgactcagaaaatgccacttgcctgtatgccaagtggcagatgaacttcactgtgagatatgagactaccaacaagacctacaagactgtgaccatctcagaccatggcactgtcacctacaatggatcaatctgtggtgatgatcagaatggcccaaagatagcagtgcagtttgggcccggtttttcctggattgctaacttcaccaaggcagcctccacctacagcattgactcagtcagcttcagctacaacactggggataacaccaccttccctgacgcagaggacaagggaatccttactgtggacgaactcctggcaatcagaatcccccttaacgacctgttcagatgcaactccctttcaacccttgaaaagaatgatgtggtgcaacactattgggacgtcctggtgcaagcctttgtgcagaatgggacagtgagtaccaacgagttcctctgtgacaaggacaagaccagcactgtggcccccactatccacaccactgtgcccagccctaccactacccccacccctaaagagaagccagaagctggaacctactcagtcaacaatggaaatgacacatgcctccttgccaccatgggactgcagctgaacatcactcaggacaaggtggcctcagtgattaacatcaaccctaacaccactcatagcactgggagctgcagatcacatacagctctgctgaggctcaactcctccaccatcaagtacctggactttgtgtttgctgtgaagaatgagaacaggttctacctcaaggaagtgaacatttccatgtacctggtcaatggttcagtgttctctattgccaacaacaatctgagctactgggatgcacccctgggatcctcctacatgtgcaacaaggagcagactgtgagtgtgtcaggtgcttttcagatcaacacttttgacctgagggtgcagcccttcaatgtgactcaggga

SEQ ID NO: 32 - DNA, 1080 bp, Artificial Sequence
  variant of LAMP-2
atggtgtgctttagactgtttcctgtgcctggttcagggctggtcctggtctgtctggtgctgggggctgtcagaagctatgccttggagctgaacctcactgatagtgaaaatgccacttgtctgtatgctaagtggcagatgaacttcactgtgagatatgaaaccaccaacaagacttacaaaacagtgaccatctcagatcatggaactgtgacctacaacggcagcatttgtggagacgaccagaacggaccaaaaatcgctgtccaatttgggcctggattctcctggattgccaatttcactaaagctgcctccacatattcaattgactcagtgtccttctcctacaacactggggacaacactactttccctgatgctgaagataagggaatcttgacagtggatgagctgctggctatcaggatccctttgaatgacctgtttaggtgtaattcactgagcactctggagaagaacgacgtggtgcagcactactgggacgtgctggtgcaggcctttgtgcagaacggcactgtgtccaccaacgaattcctgtgtgataaggacaaaacttccactgtggcacctacaattcacactactgtgccttcacctaccaccactccaactccaaaggaaaagcctgaagcaggaacctactctgtgaacaatggcaatgatacctgtctgttggccaccatgggcctccaactgaacattactcaggacaaggtggcctcagtgattaacattaaccccaacactacccactccactggcagctgtagatcacacacagccttgctcagactgaatagcagcaccatcaagtatttggattttgtgtttgcagtgaagaatgaaaacaggttctacctgaaggaagtcaacatctcaatgtacctggtgaacggctcagtgttcagcattgccaacaacaacctctcctattgggacgctccactggggagcagctacatgtgtaacaaggaacagactgtgtcagtgtcaggagccttccagattaacacctttgatctgagggtccaaccctttaatgtcactcaagga

SEQ ID NO: 33 - DNA, 1080 bp, Artificial Sequence
  variant of LAMP-2
atggtctgttttaggctgttccctgtccctggttcaggactggtcttagtgtgtctggtgcttggagctgtcagaagctatgccctggagctgaacctgactgactcagaaaatgccacttgcctgtatgccaagtggcagatgaacttcactgtcagatatgaaaccaccaacaagacctataagactgtgaccatctcagaccatggcactgtgacttacaatgggtcaatttgtggagatgaccagaatggccctaagatagctgtccagtttggtccaggattcagctggattgccaacttcaccaaggcagccagcacctacagcattgactctgtgtccttctcctacaacacaggagacaacaccactttccctgatgcagaggacaaaggtatcctgactgtggatgagttgctggcaatcaggatcccactgaacgatctgttcaggtgcaactcactgtccactctggaaaagaatgatgtggtgcagcactattgggatgtgctagtccaggcctttgtccagaatgggactgtgtcaactaatgagttcctgtgtgacaaggacaagacaagcactgtagcccccactatccataccacagtacctagccccaccactactccaacccccaaggagaagcctgaggctggcacctactcagtgaacaatgggaatgacacctgtttgctggccactatgggactccaactgaacatcacccaggacaaagtggcctctgtgatcaatatcaatcccaacaccacccacagcactgggtcctgcagaagccacactgccctcctgaggctcaactcatcaactatcaagtacttggattttgtgtttgcagtgaagaatgagaacagattctacctcaaagaggtcaacatttcaatgtacctggtgaatgggagtgtgttctccattgctaacaacaacctgagctactgggatgcccctctgggctcctcatacatgtgcaacaaggaacagactgtgagtgtgtcaggggccttccagatcaacacttttgacctgagagtgcagccctttaatgtgacacaggga

SEQ ID NO: 34 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 35 - protein, 411 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAEECSADSDLNFLIPVAVGVALGFLIIVVFISYMIGRRKSRTGYQSV

(19) United States
(12) Patent Application Publication
(10) Pub. No.: US 2024/0033325 A1
(43) Pub. Date: Feb. 1, 2024

(54) TREATMENT OF DANON DISEASE

(71) Applicant: Spacecraft Seven, LLC, Cranbury, NJ (US)

(72) Inventors: Jose M. TREVEJO, Cranbury, NJ (US); Gaurav SHAH, Cranbury, NJ (US)

(21) Appl. No.: 18/265,421

(22) PCT Filed: Dec. 7, 2021
(86) PCT No.: PCT/US2021/062112

§ 371 Date: Jun. 5, 2023

Related U.S. Application Data
(60) Provisional application No. 63/122,249, filed on Dec. 7, 2020.

(51) Int. Cl. A61K 38/17; A61K 48/00; A61K 39/395; A61K 31/436; A61K 45/06; A61P 3/00

(52) U.S. Cl. CPC A61K 38/177; A61K 48/0041; A61K 48/0083; A61K 39/3955; A61K 31/436; A61K 45/06; A61K 48/0075; A61K 48/0066; A61P 3/00

(57) ABSTRACT

Methods for treating Danon disease in a subject identified as suffering from or at risk for Danon disease and/or having an inactivating mutation in one or more isoforms of the LAMP-2 gene and provided. The methods may comprise administering to the subject a therapeutically effective amount of a recombinant adeno-associated virus (rAAV) virion comprising a capsid and a vector genome where the vector genome comprises a polynucleotide sequence encoding a LAMP-2 protein, preferably a LAMP-2B protein.

### CROSS REFERENCE TO RELATED APPLICATION

This application claims priority to U.S. Provisional Patent Application No. 63/122,249, filed Dec. 7, 2020 and titled “Treatment of Danon Disease,” which is incorporated herein by reference in its entirety.

### SEQUENCE LISTING

This application is being filed electronically via EFS-Web and includes an electronically submitted sequence listing in .txt format. The .txt file contains a sequence listing entitled “ROPA_023_01WO_SeqList_ST25.txt” created on Dec. 7, 2021 and having a size of ˜62 kilobytes. The sequence listing contained in this .txt file is part of the specification and is incorporated herein by reference in its entirety.

### FIELD OF INVENTION

The invention relates generally to clinical use of an adeno-associated virus (AAV) gene therapy in Danon Disease.

### BACKGROUND

Lysosome-associated membrane protein 2 (LAMP-2, also known as CD107b) is a gene that encodes a lysosome-associated membrane glycoprotein. Alternative splicing of the gene produces three isoforms: LAMP-2A, LAMP-2B, and LAMP-2C. Loss-of-function mutations in LAMP-2 are associated with human diseases, including Danon disease, a familial cardiomyopathy associated with impaired autophagy.

International Patent Application Publication No. WO2017127565A1 discloses that overexpression of LAMP-2 in human induced pluripotent stem cells (hiPSCs) derived from patients with LAMP-2 mutations, as described in Hashem, et al., Stem Cells. 2015 July; 33(7):2343-50, results in reduced oxidative stress levels and apoptotic cell death, confirming the importance of LAMP-2B in disease pathophysiology.

There remains a need in the art for methods and compositions related to treatment of Danon disease in human subjects. The present disclosure provides such methods of compositions.

### SUMMARY OF THE INVENTION

In an aspect, the disclosure provides a method for treating Danon disease in a subject identified as suffering from or at risk for Danon disease and/or having an inactivating mutation in one or more isoforms of the LAMP-2 gene, comprising administering to the subject a therapeutically effective amount of a recombinant adeno-associated virus (rAAV) virion comprising a capsid and a vector genome where the vector genome comprises a polynucleotide sequence encoding a LAMP-2 protein, preferably a LAMP-2B protein.

In some embodiments, the therapeutically effective amount is less than about 2×10¹⁴ vector genomes (vg) per kilogram (kg) of the subject's body weight.

In some embodiments, the therapeutically effective amount is less than about 1.5×10¹⁴ vg/kg of the subject's body weight.

In some embodiments, the therapeutically effective amount is less than about 1×10¹⁴ vg/kg of the subject's body weight.

In some embodiments, the therapeutically effective amount is at least about 1×10¹² vg/kg of the subject's body weight.

In some embodiments, the therapeutically effective amount is at least about 1×10¹³ vg/kg of the subject's body weight.

In some embodiments, the therapeutically effective amount is about 6.7×10¹³ vg/kg of the subject's body weight.

In some embodiments, the therapeutically effective amount is about 1.1×10¹⁴ vg/kg of the subject's body weight.

In some embodiments, the therapeutically effective amount is about 2.0×10¹⁴ vg/kg of the subject's body weight.

In some embodiments, the method further comprises administering to the subject an effective amount of tacrolimus.

In some embodiments, the method further comprises administering to the subject an effective amount of rituximab.

In some embodiments, the method comprises administering to the subject an effective amount of tacrolimus and administering to the subject an effective amount of rituximab.

In some embodiments, the method comprises administering to the subject an effective amount of eculizumab.

In some embodiments, the method further comprises administering to the subject an effective amount of rituximab; administering to the subject an effective amount of tacrolimus;

and/or administering to the subject an effective amount of eculizumab.

In some embodiments, the subject is at risk for sequelae of complement activation, such as atypical hemolytic-uremic syndrome (aHUS), optionally aHUS resulting in reversible thrombocytopenia and/or acute kidney injury (AKI).

In some embodiments, the method further comprises administering to the subject an effective amount of corticosteroids.

In some embodiments, the method further comprises administering to the subject an effective amount of corticosteroids prior to administering the effective amount of tacrolimus.

In some embodiments, the subject is a juvenile subject, optionally having an age of 8-14 years old and/or 15-17 years old.

In some embodiments, the subject is a pediatric subject, optionally having an age of 0-8 years old.

In some embodiments, the subject is an adult subject, optionally having an age of 18 years old or older.

In some embodiments, the therapeutically effective amount of the AAV is administered intravenously.

In some embodiments, the therapeutically effective amount of the AAV is administered by direct cardiac injection, optionally by intrajugular or Swan-Ganz catheter

In some embodiments, the therapeutically effective amount of the AAV is administered by intraperitoneal injection.

In some embodiments, the method results in one or more of: a) transduction of cardiomyocyte and/or skeletal muscle by the AAV; b) expression of exogenous ribonucleic acid polynucleotide encoding LAMP-2B and/or expression of exogenous LAMP-2B protein, optionally in cardiomyocyte and/or skeletal muscle; c) correction or improvement of one or more Danon disease-associated histologic abnormalities, optionally autophagic vacuoles or myofibrillar disarray, optionally determined by histology of sampled endomyocardial biopsy; d) correction or improvement of cardiomyocyte molecular marker expression; and/or e) correction or improvement of cardiomyocyte histology.

In some embodiments, the AAV comprises an expression cassette comprising the polynucleotide sequence encoding the LAMP-2B protein operatively linked to a promoter, and wherein the polynucleotide sequence shares at least 95% identity to SEQ ID NO: 2 and/or the LAMP-2B protein shares at least 95% identity to SEQ ID NO: 1.

In some embodiments, the polynucleotide sequence comprises or consists of SEQ ID NO: 2 and/or the LAMP-2B protein comprises or consists of SEQ ID NO: 1.

In some embodiments, the promoter is a CAG promoter.

In some embodiments, the promoter comprises an enhancer/promoter region that shares at least 95% identity to SEQ ID NO: 22.

In some embodiments, the enhancer/promoter region comprises or consists of SEQ ID NO: 22.

In some embodiments, the expression cassette comprises, in 5′ to 3′ order:
(a) an enhancer/promoter region that comprises SEQ ID NO: 22;
(b) the polynucleotide sequence that encodes the LAMP-2B protein, wherein the polynucleotide sequence comprises SEQ ID NO: 3;
(c) a 3′ UTR sequence comprising SEQ ID NO: 27; and/or
(d) a poly-adenylation sequence comprising SEQ ID NO: 7.

In some embodiments, the expression cassette is flanked by: (i) a 5′ ITR that comprises SEQ ID NO: 11; and (ii) a 3′ ITR that comprises SEQ ID NO: 12.

In some embodiments, the expression cassette comprises SEQ ID NO: 8.

In some embodiments, the capsid is an AAV9 capsid.

In some embodiments, the AAV9 capsid comprises one or more capsid proteins that comprise amino acids 1 to 736 of SEQ ID NO: 28, amino acids 138 to 736 of SEQ ID NO: 28, or amino acids 203 to 736 of SEQ ID NO: 28.

In other aspects, the disclosure provides a unit dose, pharmaceutical composition, or composition for use in treating Danon disease. The unit dose, pharmaceutical composition, or composition for use comprises a therapeutically effective amount of a recombinant adeno-associated virus (rAAV) virion comprising a capsid and a vector genome where the vector genome comprises a polynucleotide sequence encoding a LAMP-2 protein, preferably a LAMP-2B protein.

In some embodiments, the therapeutically effective amount is less than about 2×10¹⁴ vector genomes (vg) per kilogram (kg) of the subject's body weight.

In some embodiments, the therapeutically effective amount is less than about 1.5×10¹⁴ vg/kg.

In another aspect, the disclosure provides a kit comprising the unit dose, pharmaceutical composition, or composition for use of the disclosure; and instructions for use in treating Danon disease.

The kit may further comprising one or more unit dose, pharmaceutical composition, or composition comprising one or more of: rituximab; tacrolimus; eculizumab; and a corticosteroid.

Further aspects and embodiments of the invention are disclosed in the Detailed Description that follows.

### BRIEF DESCRIPTION OF THE DRAWINGS

FIG. 1 depicts a diagram of the pAAV-LAMP2B transfer plasmid used to generate the adeno-associated virus (AAV) particle in the present disclosure. The AAV particle contains an expression cassette flanked by inverted terminal repeat (ITR) elements derived from AAV2, a CAG promoter comprising of cytomegalovirus immediate early (CMV IE) enhancer, chicken beta-actin (CBA) promoter, chicken beta-actin and rabbit globin introns (CBA/RbG Intron), a LAMP2B transgene, a woodchuck hepatitis virus post-transcriptional regulatory element (WPRE), and a rabbit globin poly-adenylation signal (RGpA).

FIGS. 2A-2B depict graphs of cardiac tissue analyses in WT or LAMP2 KO mice injected with PBS or varying doses of AAV9.LAMP2B (1×10¹³, 5×10¹³, and 1×10¹⁴ vg/kg). FIG. 2A shows vector copy number per nucleus (VCN/Nuclei) determined using qPCR. FIG. 2B shows quantitative RT-PCR of LAMP2B mRNA levels (fold change over PBS, normalized by GAPDH). Primers detect both human and mouse LAMP2B mRNA. Values are Mean±SEM. ##p<0.01, ###p<0.0001 vs KO mice injected with PBS.

FIGS. 3A-3C depict graphs of human LAMP2 and LC3-II Protein Expression in heart tissue. FIG. 3A shows protein lysates from hearts of Lamp2 KO mice injected with PBS or increasing doses of AAV9.LAMP2B (1×10¹³, 5×10¹³, and 1×10¹⁴ vg/kg), and naïve control WT mice evaluated by western blot for mouse (mLAMP2) and human LAMP2 (hLAMP2), LC3-II (marker for autophagy), GAPDH used as a loading control. FIG. 3B shows the quantification of hLAMP2 protein (densitometry using ImageJ) normalized to GAPDH. FIG. 3C shows the quantification of LC3-II (densitometry using ImageJ) normalized to GAPDH. Values are Mean±SEM. **p<0.01 vs WT; #p<0.05, ##p<0.01 vs KO mice injected with PBS.

FIG. 4 depicts transmission electron microscopy images of cardiac tissues showing vacuoles (autophagy structures as denoted by yellow arrows). Representative images from WT type or Lamp2 KO mice injected with PBS, or AAV9.LAMP2B at 5×10¹³ or 1×10¹⁴ vg/kg. Scale bars are 2 μm in upper panels and 0.54 μm in lower panels.

FIG. 5 depicts a graph of Vector DNA copies per diploid genome in patients treated with 6.7×10¹³ GC/kg AAV9.LAMP2B. Patients 1001, 1002 and 1005 were analyzed at Baseline, Week 8, Month 6, and Month 12 following treatment.

FIG. 6 depicts immunohistochemistry images of cardiac tissues from patient 1002 showing cardiac LAMP2B expression. Representative images from Pre-dose, Positive Control, or patient treated with 6.7×10¹³ GC/kg AAV9.LAMP2B at Week 8, Month 6, and Month 12 following treatment. Scale bars are 100 μm.

FIGS. 7A-7C depict graphs of the fold change of Brain natriuretic peptide (BNP) over baseline for patient 1001 (FIG. 7A), patient 1002 (FIG. 7B), and patient 1005 (FIG. 7C). Patients were treated with 6.7×10¹³ GC/kg AAV9.LAMP2B at BNP fold change was analyzed at Week 8, Month 6, and Month 12 following treatment.

FIG. 8 depicts a diagram of the enrollment sequence in study cohorts, in a scenario in which no DLT is identified.

FIG. 9 depicts a diagram of the enrollment sequence in any cohort in the setting of a single DLT within a given cohort. Enrollment within a cohort will be halted if a second patient experiences DLT. The triangle/exclamation point indicates the DLT occurrence for patient 2.

FIGS. 10A-10D are charts depicting the AAV9.LAMP2B Study Assessment and Therapy Table of Events. Abbreviations: AAV9: adeno-associated virus serotype 9; ADA: anti-drug antibody; aPTT: activated partial thromboplastin time; BNP: brain natriuretic peptide; C3: complement 3; C4: complement 4; CBC: complete blood count; CK-MB: creatinine kinase-MB; D: day; d/c: discontinue; ECG: electrocardiogram; HBV: hepatitis B virus; HCV: hepatitis C virus; HIV: human immunodeficiency virus; IgG: immunoglobulin G; IgM: immunoglobulin M; IP: Investigational Product; IV: intravenous infusion; LFT: liver function tests; M: Month; MRI: magnetic resonance imaging; PMBC: peripheral blood mononuclear cell; PRO: patient-reported outcome; PT: prothrombin time; QOL: quality-of-life; sC5b-9: serum membrane attack complex; TAT: thrombin-antithrombin complex; VO2: maximal oxygen consumption; W: week.

FIGS. 11A-11B depict graphs of cardiac contractility (FIG. 11A) and cardiac relaxation (FIG. 11B) analyzed by invasive left intraventricular pressure (dP/dt max and dP/dt min, respectively) in Lamp2 KO mice injected with PBS or increasing doses of AAV9.LAMP2B (1×10¹³, 5×10¹³, and 1×10¹⁴ vg/kg) or naïve control WT mice.

FIG. 12 depicts transmission electron microscopy images of cardiac tissues showing vacuoles (autophagy structures as denoted by yellow arrows). Representative images from WT type or Lamp2 KO mice injected with PBS, or AAV9.LAMP2B at 5×10¹³, 1×10¹⁴ vg/kg or 2×10¹⁴ vg/kg. Scale bars are 2 μm in upper panels and 0.6 μm in lower panels.

FIGS. 13A-13B depict graphs of cardiac contractility (FIG. 13A) and cardiac relaxation (FIG. 13B) analyzed by invasive left hemodynamics (dP/dt max and dP/dt min, respectively) in Lamp2 KO mice injected with PBS or increasing doses of AAV9.LAMP2B (1×10¹³, 5×10¹³, 1×10¹⁴, and 2×10¹⁴ vg/kg) or naïve control WT mice.

FIGS. 14A-14B depict LAMP2 protein expression by immunohistochemistry (FIG. 14A) and cell morphology by electron microscopy (FIG. 14B) after treatment with RP-A501 at low dose. Representative images from patient 1005 from biopsy of intra-ventricular septum are shown.

FIG. 15A depicts remodeling of ventricular hypertrophy on echocardiography with reduction or stabilization of wall thickness in both low and high dose patients. All echocardiographic parameters are from local laboratory assessment, conducted by a single reader.

FIG. 15B depicts stabilization or improvement of left ventricular (LV) ejection fraction (EF) and wall thickness in both low and high dose patients. All echocardiographic parameters are from local laboratory assessment, conducted by a single reader.

FIG. 15C depicts invasive hemodynamics demonstrated long term stabilization or improvement of diastolic dysfunction (LV filling pressure) as measured by pulmonary capillary wedge pressure in the low and high dose patients.

FIG. 15D shows hemodynamic stabilization or improvement of systolic function in both high and low dose patients.

### DETAILED DESCRIPTION OF THE INVENTION

The present disclosure provides methods and compositions that relate to treating Danon disease in human subjects. The present inventors have demonstrated successful treatment of Danon disease in human subjects with an adeno-associated virus (AAV) designed to expression the LAMP-2B isoform of LAMP-2. The AAV may be administered in conjunction with treatment with corticosteroids, tacrolimus, rituximab, and/or eculizumab; and the AAV may be administered at various doses. As disclosed herein, doses in a range of approximately 6.7×10¹³ vg/kg, or lower, to approximately 2.0×10¹⁴ vg/kg, or higher, may be safe and effective in Danon disease subjects.

### Vector Sequences

The wild-type polypeptide sequence of human LAMP-2B (SEQ ID NO: 1) and the wild-type polynucleotide sequence encoding human LAMP-2B (SEQ ID NO: 2) are, respectively:

| (SEQ ID NO: 1) | |
|---|---|
| 1 | MVCFRLFPVP GSGLVLVCLV LGAVRSYALE LNLTDSENAT CLYAKWQMNF TVRYETTNKT |
| 61 | YKTVTISDHG TVTYNGSICG DDQNGPKIAV QFGPGFSWIA NFTKAASTYS IDSVSFSYNT |
| 121 | GDNTTFPDAE DKGILTVDEL LAIRIPLNDL FRCNSLSTLE KNDVVQHYWD VLVQAFVQNG |
| 181 | TVSTNEFLCD KDKTSTVAPT IHTTVPSPTT TPTPKEKPEA GTYSVNNGND TCLLATMGLQ |
| 241 | LNITQDKVAS VININPNTTH STGSCRSHTA LLRLNSSTIK YLDFVFAVKN ENRFYLKEVN |
| 301 | ISMYLVNGSV FSIANNNLSY WDAPLGSSYM CNKEQTVSVS GAFQINTFDL RVQPFNVTQG |
| 361 | KYSTAQECSL DDDTILIPII VGAGLSGLII VIVIAYVIGR RKSYAGYQT; |
| and | |
| (SEQ ID NO: 2) | |
| 1 | ATGGTGTGCT TCCGCCTCTT CCCGGTTCCG GGCTCAGGGC TCGTTCTGGT CTGCCTAGTC |
| 61 | CTGGGAGCTG TGCGGTCTTA TGCATTGGAA CTTAATTTGA CAGATTCAGA AAATGCCACT |
| 121 | TGCCTTTATG CAAAATGGCA GATGAATTTC ACAGTTCGCT ATGAAACTAC AAATAAAACT |
| 181 | TATAAAACTG TAACCATTTC AGACCATGGC ACTGTGACAT ATAATGGAAG CATTTGTGGG |
| 241 | GATGATCAGA ATGGTCCCAA AATAGCAGTG CAGTTCGGAC CTGGCTTTTC CTGGATTGCG |
| 301 | AATTTTACCA AGGCAGCATC TACTTATTCA ATTGACAGCG TCTCATTTTC CTACAACACT |
| 361 | GGTGATAACA CAACATTTCC TGATGCTGAA GATAAAGGAA TTCTTACTGT TGATGAACTT |
| 421 | TTGGCCATCA GAATTCCATT GAATGACCTT TTTAGATGCA ATAGTTTATC AACTTTGGAA |
| 481 | AAGAATGATG TTGTCCAACA CTACTGGGAT GTTCTTGTAC AAGCTTTTGT CCAAAATGGC |
| 541 | ACAGTGAGCA CAAATGAGTT CCTGTGTGAT AAAGACAAAA CTTCAACAGT GGCACCCACC |
| 601 | ATACACACCA CTGTGCCATC TCCTACTACA ACACCTACTC CAAAGGAAAA ACCAGAAGCT |
| 661 | GGAACCTATT CAGTTAATAA TGGCAATGAT ACTTGTCTGC TGGCTACCAT GGGGCTGCAG |
| 721 | CTGAACATCA CTCAGGATAA GGTTGCTTCA GTTATTAACA TCAACCCCAA TACAACTCAC |
| 781 | TCCACAGGCA GCTGCCGTTC TCACACTGCT CTACTTAGAC TCAATAGCAG CACCATTAAG |
| 841 | TATCTAGACT TTGTCTTTGC TGTGAAAAAT GAAAACCGAT TTTATCTGAA GGAAGTGAAC |
| 901 | ATCAGCATGT ATTTGGTTAA TGGCTCCGTT TTCAGCATTG CAAATAACAA TCTCAGCTAC |
| 961 | TGGGATGCCC CCCTGGGAAG TTCTTATATG TGCAACAAAG AGCAGACTGT TTCAGTGTCT |
| 1021 | GGAGCATTTC AGATAAATAC CTTTGATCTA AGGGTTCAGC CTTTCAATGT GACACAAGGA |
| 1081 | AAGTATTCTA CAGCCCAAGA GTGTTCGCTG GATGATGACA CCATTCTAAT CCCAATTATA |
| 1141 | GTTGGTGCTG GTCTTTCAGG CTTGATTATC GTTATAGTGA TTGCTTACGT AATTGGCAGA |
| 1201 | AGAAAAAGTT ATGCTGGATA TCAGACTCTG TAA. |

Disclosed herein are modified polynucleotide sequences encoding an isoform of lysosome-associated membrane protein 2 (LAMP-2) or a functional variant thereof. In certain embodiments, the modified polynucleotide sequences comprise one or more of the following modifications as compared to the wild-type polynucleotide encoding the isoform of LAMP-2: codon-optimization, CpG depletion, removal of cryptic splice sites, or a reduced number of alternative open-reading frames (ORFs). In some embodiments, the modified polynucleotide encodes LAMP-2A, LAMP-2B, LAMP-2C or a functional variant of any of these isoforms. In embodiments, the disclosure provides a polynucleotide sequence or transgene encoding LAMP-2B or a functional variant thereof comprising one or more nucleotide substitutions as compared to SEQ ID NO:2. In embodiments, the transgene shares at least 85%, 86%, 87%, 88%, 89%, 90%, 91%, 92%, 93%, 94%, 95%, 96%, 97%, 98%, 99%, or complete identity to a sequence selected from SEQ ID NOs: 3-5. The disclosure provides at least three illustrative variant transgene sequences encoding LAMP-2B (SEQ ID NOs: 3-5):

| (SEQ ID NO: 3) | |
|---|---|
| 1 | ATGGTCTGCT TCAGACTGTT CCCTGTCCCT GGATCTGGTC TGGTGCTTGT GTGCTTGGTG |
| 61 | CTGGGTGCTG TGAGATCCTA TGCCCTTGAG CTGAACCTGA CTGACTCAGA AAATGCCACT |
| 121 | TGCCTGTATG CCAAGTGGCA GATGAACTTC ACTGTGAGAT ATGAGACTAC CAACAAGACC |
| 181 | TACAAGACTG TGACCATCTC AGACCATGGC ACTGTCACCT ACAATGGATC AATCTGTGGT |
| 241 | GATGATCAGA ATGGCCCAAA GATAGCAGTG CAGTTTGGGC CCGGTTTTTC CTGGATTGCT |
| 301 | AACTTCACCA AGGCAGCCTC CACCTACAGC ATTGACTCAG TCAGCTTCAG CTACAACACT |
| 361 | GGGGATAACA CCACCTTCCC TGACGCAGAG GACAAGGGAA TCCTTACTGT GGACGAACTC |
| 421 | CTGGCAATCA GAATCCCCCT TAACGACCTG TTCAGATGCA ACTCCCTTTC AACCCTTGAA |
| 481 | AAGAATGATG TGGTGCAACA CTATTGGGAC GTCCTGGTGC AAGCCTTTGT GCAGAATGGG |
| 541 | ACAGTGAGTA CCAACGAGTT CCTCTGTGAC AAGGACAAGA CCAGCACTGT GGCCCCCACT |
| 601 | ATCCACACCA CTGTGCCCAG CCCTACCACT ACCCCCACCC CTAAAGAGAA GCCAGAAGCT |
| 661 | GGAACCTACT CAGTCAACAA TGGAAATGAC ACATGCCTCC TTGCCACCAT GGGACTGCAG |
| 721 | CTGAACATCA CTCAGGACAA GGTGGCCTCA GTGATTAACA TCAACCCTAA CACCACTCAT |
| 781 | AGCACTGGGA GCTGCAGATC ACATACAGCT CTGCTGAGGC TCAACTCCTC CACCATCAAG |
| 841 | TACCTGGACT TTGTGTTTGC TGTGAAGAAT GAGAACAGGT TCTACCTCAA GGAAGTGAAC |
| 901 | ATTTCCATGT ACCTGGTCAA TGGTTCAGTG TTCTCTATTG CCAACAACAA TCTGAGCTAC |
| 961 | TGGGATGCAC CCCTGGGATC CTCCTACATG TGCAACAAGG AGCAGACTGT GAGTGTGTCA |
| 1021 | GGTGCTTTTC AGATCAACAC TTTTGACCTG AGGGTGCAGC CCTTCAATGT GACTCAGGGA |
| 1081 | AAGTACTCCA CTGCACAAGA GTGTTCCTTG GATGATGACA CTATCCTCAT CCCCATTATT |
| 1141 | GTGGGAGCTG GACTGTCAGG ATTGATTATA GTGATTGTGA TTGCTTATGT GATTGGAAGG |
| 1201 | AGAAAGAGCT ATGCTGGCTA CCAGACCCTG TAA; |
| (SEQ ID NO: 4) | |
| 1 | ATGGTGTGCT TTAGACTGTT TCCTGTGCCT GGTTCAGGGC TGGTCCTGGT CTGTCTGGTG |
| 61 | CTGGGGGCTG TCAGAAGCTA TGCCTTGGAG CTGAACCTCA CTGATAGTGA AAATGCCACT |
| 121 | TGTCTGTATG CTAAGTGGCA GATGAACTTC ACTGTGAGAT ATGAAACCAC CAACAAGACT |
| 181 | TACAAAACAG TGACCATCTC AGATCATGGA ACTGTGACCT ACAACGGCAG CATTTGTGGA |
| 241 | GACGACCAGA ACGGACCAAA AATCGCTGTC CAATTTGGGC CTGGATTCTC CTGGATTGCC |
| 301 | AATTTCACTA AAGCTGCCTC CACATATTCA ATTGACTCAG TGTCCTTCTC CTACAACACT |
| 361 | GGGGACAACA CTACTTTCCC TGATGCTGAA GATAAGGGAA TCTTGACAGT GGATGAGCTG |
| 421 | CTGGCTATCA GGATCCCTTT GAATGACCTG TTTAGGTGTA ATTCACTGAG CACTCTGGAG |
| 481 | AAGAACGACG TGGTGCAGCA CTACTGGGAC GTGCTGGTGC AGGCCTTTGT GCAGAACGGC |
| 541 | ACTGTGTCCA CCAACGAATT CCTGTGTGAT AAGGACAAAA CTTCCACTGT GGCACCTACA |
| 601 | ATTCACACTA CTGTGCCTTC ACCTACCACC ACTCCAACTC CAAAGGAAAA GCCTGAAGCA |
| 661 | GGAACCTACT CTGTGAACAA TGGCAATGAT ACCTGTCTGT TGGCCACCAT GGGCCTCCAA |
| 721 | CTGAACATTA CTCAGGACAA GGTGGCCTCA GTGATTAACA TTAACCCCAA CACTACCCAC |
| 781 | TCCACTGGCA GCTGTAGATC ACACACAGCC TTGCTCAGAC TGAATAGCAG CACCATCAAG |
| 841 | TATTTGGATT TTGTGTTTGC AGTGAAGAAT GAAAACAGGT TCTACCTGAA GGAAGTCAAC |
| 901 | ATCTCAATGT ACCTGGTGAA CGGCTCAGTG TTCAGCATTG CCAACAACAA CCTCTCCTAT |
| 961 | TGGGACGCTC CACTGGGGAG CAGCTACATG TGTAACAAGG AACAGACTGT GTCAGTGTCA |
| 1021 | GGAGCCTTCC AGATTAACAC CTTTGATCTG AGGGTCCAAC CCTTTAATGT CACTCAAGGA |
| 1081 | AAGTATAGCA CTGCCCAGGA GTGCTCCCTG GATGATGACA CCATTCTGAT TCCAATCATT |
| 1141 | GTGGGTGCAG GACTTTCTGG GCTTATTATT GTGATTGTGA TTGCCTATGT GATTGGCAGA |
| 1201 | AGGAAATCCT ATGCAGGGTA CCAAACTCTG TAA; |
| and | |
| (SEQ ID NO: 5) | |
| 1 | ATGGTCTGTT TTAGGCTGTT CCCTGTCCCT GGTTCAGGAC TGGTCTTAGT GTGTCTGGTG |
| 61 | CTTGGAGCTG TCAGAAGCTA TGCCCTGGAG CTGAACCTGA CTGACTCAGA AAATGCCACT |
| 121 | TGCCTGTATG CCAAGTGGCA GATGAACTTC ACTGTCAGAT ATGAAACCAC CAACAAGACC |
| 181 | TATAAGACTG TGACCATCTC AGACCATGGC ACTGTGACTT ACAATGGGTC AATTTGTGGA |
| 241 | GATGACCAGA ATGGCCCTAA GATAGCTGTC CAGTTTGGTC CAGGATTCAG CTGGATTGCC |
| 301 | AACTTCACCA AGGCAGCCAG CACCTACAGC ATTGACTCTG TGTCCTTCTC CTACAACACA |
| 361 | GGAGACAACA CCACTTTCCC TGATGCAGAG GACAAAGGTA TCCTGACTGT GGATGAGTTG |
| 421 | CTGGCAATCA GGATCCCACT GAACGATCTG TTCAGGTGCA ACTCACTGTC CACTCTGGAA |
| 481 | AAGAATGATG TGGTGCAGCA CTATTGGGAT GTGCTAGTCC AGGCCTTTGT CCAGAATGGG |
| 541 | ACTGTGTCAA CTAATGAGTT CCTGTGTGAC AAGGACAAGA CAAGCACTGT AGCCCCCACT |
| 601 | ATCCATACCA CAGTACCTAG CCCCACCACT ACTCCAACCC CCAAGGAGAA GCCTGAGGCT |
| 661 | GGCACCTACT CAGTGAACAA TGGGAATGAC ACCTGTTTGC TGGCCACTAT GGGACTCCAA |
| 721 | CTGAACATCA CCCAGGACAA AGTGGCCTCT GTGATCAATA TCAATCCCAA CACCACCCAC |
| 781 | AGCACTGGGT CCTGCAGAAG CCACACTGCC CTCCTGAGGC TCAACTCATC AACTATCAAG |
| 841 | TACTTGGATT TTGTGTTTGC AGTGAAGAAT GAGAACAGAT TCTACCTCAA AGAGGTCAAC |
| 901 | ATTTCAATGT ACCTGGTGAA TGGGAGTGTG TTCTCCATTG CTAACAACAA CCTGAGCTAC |
| 961 | TGGGATGCCC CTCTGGGCTC CTCATACATG TGCAACAAGG AACAGACTGT GAGTGTGTCA |
| 1021 | GGGGCCTTCC AGATCAACAC TTTTGACCTG AGAGTGCAGC CCTTTAATGT GACACAGGGA |
| 1081 | AAGTACAGCA CTGCTCAGGA GTGCAGCCTG GATGATGACA CTATCCTGAT CCCTATCATT |
| 1141 | GTGGGGGCAG GCCTGTCTGG ACTCATTATT GTGATTGTGA TTGCCTATGT GATAGGGAGA |
| 1201 | AGGAAGTCTT ATGCTGGATA CCAGACCCTG TAA. |

In an embodiment, the transgene shares at least 95% identity to a sequence selected from SEQ ID NOs: 2-5. In an embodiment, the transgene shares at least 99% identity to a sequence selected from SEQ ID NOs: 2-5. In an embodiment, the transgene comprises a sequence selected from SEQ ID NOs: 2-5. In an embodiment, the transgene shares at least 85%, 86%, 87%, 88%, 89%, 90%, 91%, 92%, 93%, 94%, 95%, 96%, 97%, 98%, 99%, or complete identity to SEQ ID NO: 3. In an embodiment, the transgene shares at least 85%, 86%, 87%, 88%, 89%, 90%, 91%, 92%, 93%, 94%, 95%, 96%, 97%, 98%, 99%, or complete identity to SEQ ID NO: 4. In an embodiment, the transgene shares at least 85%, 86%, 87%, 88%, 89%, 90%, 91%, 92%, 93%, 94%, 95%, 96%, 97%, 98%, 99%, or complete identity to SEQ ID NO: 5.

In some embodiments, the transgene is similar to or identical to a subsequence of any one of SEQ ID NOs: 2-5. In some embodiments, the transgene comprises a subsequence of any one of SEQ ID NOs: 2-5. In various embodiments, the subsequence may comprise any set of consecutive nucleotides (nt) in the full sequence having a length of at least about 50 nt, at least about 100 nt, at least about 150 nt, at least about 250 nt, at least about 200 nt, at least about 350 nt, at least about 450 nt, at least about 400 nt, at least about 450 nt, at least about 550 nt, at least about 600 nt, at least about 650 nt, at least about 600 nt, at least about 650 nt, at least about 700 nt, at least about 750 nt, at least about 800 nt, at least about 850 nt, at least about 900 nt, at least about 950 nt, or at least about 1000 nt.

In some embodiments, the transgene shares at least 95% identity to a subsequence that comprises nucleotides 1-500, 250-750, 500-1000, or 750-1240 of any one of SEQ ID NO: 3-5. In an embodiment, the transgene shares at least 99% identity to a subsequence that comprises nucleotides 1-500, 250-750, 500-1000, or 750-1240 of any one of SEQ ID NO: 3-5. In an embodiment, the transgene comprises a sequence that comprises nucleotides 1-500, 250-750, 500-1000, or 750-1240 of any one of SEQ ID NOs: 2-5. In embodiment, the transgene shares at least 85%, 86%, 87%, 88%, 89%, 90%, 91%, 92%, 93%, 94%, 95%, 96%, 97%, 98%, 99%, or complete identity to a subsequence that comprises nucleotides 1-500, 250-750, 500-1000, or 750-1240 of any one of SEQ ID NOs: 2-5. In embodiments, the transgene shares at least 85%, 86%, 87%, 88%, 89%, 90%, 91%, 92%, 93%, 94%, 95%, 96%, 97%, 98%, 99%, or complete identity to a subsequence that comprises nucleotides 1-500, 250-750, 500-1000, or 750-1240 of SEQ ID NO: 3. In embodiment, the transgene shares at least 85%, 86%, 87%, 88%, 89%, 90%, 91%, 92%, 93%, 94%, 95%, 96%, 97%, 98%, 99%, or complete identity to a subsequence that comprises nucleotides 1-500, 250-750, 500-1000, or 750-1240 of SEQ ID NO: 3.

In certain embodiments, the transgene encodes any of the various isoforms of LAMP-2, including any of LAMP-2A, LAMP-2B, or LAMP-2C, or a functional fragment or variant of any of these isoforms. Thus, in particular embodiments, the expression cassette is an optimized polynucleotide sequence encoding any of LAMP-2A, LAMP-2B, or LAMP-2C, or a functional fragment or variant thereof, which comprises one or more modifications as compared to the corresponding wild-type polynucleotide sequence, including one or more modification selected from: codon-optimization of the transgene sequence encoding LAMP-2A, LAMP-2B, or LAMP-2C; the expression cassette or transgene sequence contains fewer CpG sites than its corresponding wild-type sequence; the expression cassette or transgene sequence contains fewer CpG sites than its corresponding wild-type sequence; the expression cassette or transgene sequence contains fewer cryptic splice sites than its corresponding wild-type sequence; and/or the expression cassette or transgene sequence contains fewer open reading frames than its corresponding wild-type sequence. In particular embodiments, the optimized sequence is optimized for increased expression in human cells. The wild-type human polynucleotide sequences encoding the LAMP-2A and LAMP-2C isoforms are set forth in SEQ ID NOs: 29 and 30, respectively. The wild-type sequences of human LAMP-2A and LAMP-2C proteins are set forth in SEQ ID NOs: 34 and 35, respectively. The sequences of the wild-type LAMP-2 isoforms and coding sequences are also publicly available. While the specification describes specific embodiments with respect to LAMP-2B, it is understood that LAMP-2A or LAMP-2C could alternatively be used in each embodiment.

The coding sequences of wild-type LAMP-2A (SEQ ID NO: 29) and wild-type LAMP-2C (SEQ ID NO: 30) are 100% identical to the coding sequence of wild-type LAMP-2B (SEQ ID NO: 2) across at least nucleotides 1-1080. Accordingly, it will be readily recognized by those in the art that that transgenes, expression cassettes, and vectors disclosed herein can be adapted for expression of these isoforms of LAMP-2 by substituting the 3′ end (nucleotides 1081—end) of either of LAMP-2A (SEQ ID NO: 29) or wild-type LAMP-2C (SEQ ID NO: 30) in place of nucleotides 1081-1233 of LAMP-2B (e.g., an optimized LAMP-2B of any of SEQ ID NO: 3-5). For example, embodiments of the invention utilize nucleotides 1-1080 of the optimized LAMP-2B gene sequences, SEQ ID NOs: 3-5, which are, respectively, SEQ ID NOs: 31-33.

In an embodiment, the transgene shares at least 95% identity to a sequence selected from SEQ ID NOs: 31-33. In an embodiment, the transgene shares at least 99% identity to a sequence selected from SEQ ID NOs: 31-33. In an embodiment, the transgene comprises a sequence selected from SEQ ID NOs: 31-33. In an embodiment, the transgene shares at least 85%, 86%, 87%, 88%, 89%, 90%, 91%, 92%, 93%, 94%, 95%, 96%, 97%, 98%, 99%, or complete identity to SEQ ID NO: 31. In an embodiment, the transgene shares at least 85%, 86%, 87%, 88%, 89%, 90%, 91%, 92%, 93%, 94%, 95%, 96%, 97%, 98%, 99%, or complete identity to SEQ ID NO: 32. In an embodiment, the transgene shares at least 85%, 86%, 87%, 88%, 89%, 90%, 91%, 92%, 93%, 94%, 95%, 96%, 97%, 98%, 99%, or complete identity to SEQ ID NO: 33. In some cases, the transgene has a polynucleotide sequence that is different from the polynucleotide sequence of a reference sequence, e.g., a “native” or “wild-type” LAMP-2B sequence. In some embodiments, the transgene shares at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% identity with a reference sequence. In some embodiments, the reference sequence is SEQ ID NO: 2. For example, SEQ ID NO: 3 shares 78.5% identity to SEQ ID NO: 2.

In some cases, the transgene has a polynucleotide sequence that is different from the polynucleotide sequence of a reference sequence, e.g., a “native” or “wild-type” LAMP-2A sequence. In some embodiments, the transgene shares at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% identity with a reference sequence. In some embodiments, the reference sequence is the wild-type human LAMP-2A sequence set forth in SEQ ID NO: 29.

In some cases, the transgene has a polynucleotide sequence that is different from the polynucleotide sequence of a reference sequence, e.g., a “native” or “wild-type” LAMP-2C sequence. In some embodiments, the transgene shares at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% identity with a reference sequence. In some embodiments, the reference sequence is the wild-type human LAMP-2C sequence set forth in SEQ ID NO: 30.

In an embodiment, the transgene is codon-optimized for expression in a human host cell. In an embodiment, the transgene coding sequence is modified, or “codon optimized” to enhance expression by replacing infrequently represented codons with more frequently represented codons. The coding sequence is the portion of the mRNA sequence that encodes the amino acids for translation. During translation, each of 61 trinucleotide codons are translated to one of 20 amino acids, leading to a degeneracy, or redundancy, in the genetic code. However, different cell types, and different animal species, utilize tRNAs (each bearing an anticodon) coding for the same amino acids at different frequencies. When a gene sequence contains codons that are infrequently represented by the corresponding tRNA, the ribosome translation machinery may slow, impeding efficient translation. Expression can be improved via “codon optimization” for a particular species, where the coding sequence is altered to encode the same protein sequence, but utilizing codons that are highly represented, and/or utilized by highly expressed human proteins (Cid-Arregui et al., 2003; J. Virol. 77: 4928).

In some embodiments, the coding sequence of the transgene is modified to replace codons infrequently expressed in mammal or in primates with codons frequently expressed in primates. For example, in some embodiments, the transgene encodes a polypeptide having at least 85% sequence identity to a reference polypeptide (e.g. wild-type LAMP-2B; SEQ ID NO: 3)—for example, at least 90% sequence identity, at least 95% sequence identity, at least 98% identity, or at least 99% identity to the reference polypeptide—wherein at least one codon of the coding sequence has a higher tRNA frequency in humans than the corresponding codon in the sequence disclosed above or herein.

In an embodiment, the transgene comprises fewer alternative open reading frames than SEQ ID: 2. In an embodiment, the transgene is modified to enhance expression by termination or removal of open reading frames (ORFs) that do not encode the desired transgene. An open reading frame (ORF) is the nucleic acid sequence that follows a start codon and does not contain a stop codon. ORFs may be in the forward or reverse orientation, and may be “in frame” or “out of frame” compared with the gene of interest. Such open reading frames have the potential to be expressed in an expression cassette alongside the gene of interest, and could lead to undesired adverse effects. In some embodiments the transgene has been modified to remove open reading frames by further altering codon usage. This may be done by eliminating one or more start codons (ATG) and/or introducing one or more stop codons (TAG, TAA, or TGA) in reverse orientation or out-of-frame to the desired ORF, while preserving the encoded amino acid sequence and, optionally, maintaining highly utilized codons in the gene of interest (i.e., avoiding codons with frequency <20%).

In some embodiments, the expression cassette comprises at most one, at most two, at most three, at most four, or at most five start codons 5′ to the start codon of the transgene. In some embodiments, the expression cassette comprises no start codon 5′ to the start codon of the transgene. In some embodiments, one or more ATG codons in the 5′ UTR, the promoter, the enhance, the promoter/enhancer element, or other sequences 5′ to the start codon of the transgene remain after one or more cryptic start sites are removed. In some embodiments, the expression cassette comprises no cryptic starts sites upstream of transgene to generate erroneous mRNAs.

In variations of the present disclosure, the transgene coding sequence may be optimized by either codon optimization or removal of non-transgene ORFs or using both techniques. In some cases, one removes or minimizes non-transgene ORFs after codon optimization in order to remove ORFs introduced during codon optimization.

In an embodiment, the transgene contains fewer CpG sites than SEQ ID: 2. Without being bound by theory, it is believed that the presence of CpG sites in a polynucleotide sequence is associated with the undesirable immunological responses of the host against a viral vector comprising the polynucleotide sequence. In some embodiments, the transgene is designed to reduce the number of CpG sites. Exemplary methods are provides in U.S. Patent Application Publication No. US20020065236A1.

In an embodiment, the transgene contains fewer cryptic splice sites than SEQ ID: 2. For the optimization, GeneArt® software may be used, e.g., to increase the GC content and/or remove cryptic splice sites in order to avoid transcriptional silencing and, therefore, increase transgene expression. Alternatively, any optimization method known in the art may be used. Removal of cryptic splice sites is described, for example, in International Patent Application Publication No. WO2004015106A1.

Also disclosed herein are expression cassettes and gene therapy vectors encoding LAMP-2B. In certain embodiments, the expression cassettes and gene therapy vectors comprise a codon-optimized or variant LAMP-2B polynucleotide sequence or transgene sequence disclosed herein.

In particular embodiments, an expression cassette or gene therapy vector encoding LAMP-2B comprises: a consensus optimal Kozak sequence, a full-length polyadenylation (polyA) sequence (or substitution of full-length polyA by a truncated polyA), and minimal or no upstream (i.e. 5′) or cryptic start codons (i.e. ATG sites). In some embodiments, the expression cassette comprises no start site 5′ to the transgene capable of generating alternative mRNAs. In certain embodiments, the expression cassette or gene therapy vector comprises a sequence encoding LMAP-2B, e.g., a codon-optimized or variant LAMP-2B polynucleotide sequence or transgene sequence disclosed herein.

In some cases, the expression cassette contains two or more of a first inverted terminal repeat, an enhancer/promoter region, a consensus optimal Kozak sequence, a transgene (e.g., a transgene encoding a LAMP-2B disclosed herein), a 3′ untranslated region including a full-length polyA sequence, and a second inverted terminal repeat. In some embodiments, one or both of the inverted terminal repeats (ITRs) are AAV1, AAV2, AAV3, AAV4, AAV5, AAV6, AAV7, or AAV9 ITRs, or any one ITR known in the art. In some embodiments, the expression cassette comprises exactly two ITRs. In some embodiments, both ITRs are AAV2, AAV5, or AAV9 ITRs. In some embodiments, both ITRs are AAV2 ITRs.

In an embodiment, the expression cassette comprises a Kozak sequence operatively linked to the transgene. In an embodiment, the Kozak sequence is a consensus optimal Kozak sequence comprising or consisting of SEQ ID NO: 6:

(SEQ ID NO: 6)
GCCGCCACCATGG.

In various embodiments, the expression cassette comprises an alternative Kozak sequence operatively linked to the transgene. In an embodiment, the Kozak sequence is an alternative Kozak sequence comprising or consisting of any one of SEQ ID NOs. 14-18:

(SEQ ID NO: 14)
(gcc)gccRccAUGG;
(SEQ ID NO: 15)
AGNNAUGN;
(SEQ ID NO: 16)
ANNAUGG;
(SEQ ID NO: 17)
ACCAUGG;
(SEQ ID NO: 18)
GACACCAUGG.

In some embodiments, the expression cassette comprises no Kozak sequence.

In SEQ ID NO: 14, a lower-case letter denotes the most common base at a position where the base can nevertheless vary; an upper-case letter indicates a highly conserved base; R indicates adenine or guanine. In SEQ ID NO: 14, the sequence in parentheses (GCC) is optional. In SEQ ID NOs: 15-17, ‘N’ denotes any base.

A variety of sequences can be used in place of this consensus optimal Kozak sequence as the translation-initiation site and it is within the skill of those in the art to identify and test other sequences. See Kozak M. An analysis of vertebrate mRNA sequences: intimations of translational control. J. Cell Biol. 115 (4): 887-903 (1991).

In an embodiment, the expression cassette comprises a full-length polyA sequence operatively linked to the transgene. In an embodiment, the full-length polyA sequence comprises SEQ ID NO: 7:

| (SEQ ID NO: 7) | |
|---|---|
| 1 | TGGCTAATAA AGGAAATTTA TTTTCATTGC AATAGTGTGT TGGAATTTTT TGTGTCTCTC |
| 61 | ACTCGGAAGG ACATATGGGA GGGCAAATCA TTTAAAACAT CAGAATGAGT ATTTGGTTTA |
| 121 | GAGTTTGGCA ACATATGCCC ATATGCTGGC TGCCATGAAC AAAGGTTGGC TATAAAGAGG |
| 181 | TCATCAGTAT ATGAAACAGC CCCCTGCTGT CCATTCCTTA TTCCATAGAA AAGCCTTGAC |
| 241 | TTGAGGTTAG ATTTTTTTTA TATTTTGTTT TGTGTTATTT TTTTCTTTAA CATCCCTAAA |
| 301 | ATTTTCCTTA CATGTTTTAC TAGCCAGATT TTTCCTCCTC TCCTGACTAC TCCCAGTCAT |
| 361 | AGCTGTCCCT CTTCTCTTAT GGAGATC. |

Various alternative polyA sequences may be used in expression cassettes of the present disclosure, including without limitation, bovine growth hormone polyadenylation signal (bGHpA) (SEQ ID NO: 19), the SV40 early/late polyadenylation signal (SEQ ID NO: 20), and human growth hormone (HGH) polyadenylation signal (SEQ ID NO: 21):

| (SEQ ID NO: 19) | |
|---|---|
| 1 | TCGACTGTGC CTTCTAGTTG CCAGCCATCT GTTGTTTGCC CCTCCCCCGT GCCTTCCTTG |
| 61 | ACCCTGGAAG GTGCCACTCC CACTGTCCTT TCCTAATAAA ATGAGGAAAT TGCATCGCAT |
| 121 | TGTCTGAGTA GGTGTCATTC TATTCTGGGG GGTGGGGTGG GGCAGGACAG CAAGGGGGAG |
| 181 | GATTGGGAGG ACAATAGCAG GCATGCTGGG GATGCGGTGG GCTCTATGGC TTCTG; |
| (SEQ ID NO: 20) | |
| 1 | CAGACATGAT AAGATACATT GATGAGTTTG GACAAACCAC AACTAGAATG CAGTGAAAAA |
| 61 | AATGCTTTAT TTGTGAAATT TGTGATGCTA TTGCTTTATT TGTAACCATT ATAAGCTGCA |
| 121 | ATAAACAAGT TAACAACAAC AATTGCATTC ATTTTATGTT TCAGGTTCAG GGGGAGATGT |
| 181 | GGGAGGTTTT TTAAAGCAAG TAAAACCTCT ACAAATGTGG TA; |
| (SEQ ID NO: 21) | |
| 1 | CTGCCCGGGT GGCATCCCTG TGACCCCTCC CCAGTGCCTC TCCTGGCCCT GGAAGTTGCC |
| 61 | ACTCCAGTGC CCACCAGCCT TGTCCTAATA AAATTAAGTT GCATCATTTT GTCTGACTAG |
| 121 | GTGTCCTTCT ATAATATTAT GGGGTGGAGG GGGGTGGTAT GGAGCAAGGG GCCCAAGTTG |
| 181 | GGAAGAAACC TGTAGGGCCT GC. |

In some embodiments, the expression cassette comprises an active fragment of a polyA sequence. In particular embodiments, the active fragment of the polyA sequence comprises or consists of less than 20 base pair (bp), less than 50 bp, less than 100 bp, or less than 150 bp, e.g., of any of the polyA sequences disclosed herein.

In some cases, expression of the transgene is increased by ensuring that the expression cassette does not contain competing ORFs. In an embodiment, the expression cassette comprises no start codon within 20, 30, 40, 50, 60, 70, 80, 90, 100, 200, 300, 400, or 500 base pairs 5′ of the start codon of the transgene. In some embodiment, the expression cassette comprises no start codon 5′ of the start codon of the transgene. In some embodiments, the expression cassette comprises no start site 5′ to the transgene capable of generating alternative mRNAs.

In an embodiment, the expression cassette comprises operatively linked, in the 5′ to 3′ direction, a first inverted terminal repeat, an enhancer/promoter region, introns, a consensus optimal Kozak sequence, the transgene, a 3′ untranslated region including a full-length polyA sequence, and a second inverted terminal repeat, wherein the expression cassette comprises no start site 5′ to the transgene capable of generating alternative mRNAs.

In some embodiments, the enhancer/promoter region comprises, in the 5′ to 3′ direction: a CMV IE enhancer and a chicken beta-actin promoter. In an embodiment, the enhancer/promoter region comprises a CAG promoter (SEQ ID NO: 22). As used herein “CAG promoter” refers to a polynucleotide sequence comprising a CMV early enhancer element, a chicken beta-actin promoter, the first exon and first intron of the chicken beta-actin gene, and a splice acceptor from the rabbit beta-globin gene.

| | |
|---|---|
| 1 | CTAGTCGACA TTGATTATTG ACTAGTTATT AATAGTAATC AATTACGGGG TCATTAGTTC |
| 61 | ATAGCCCATA TATGGAGTTC CGCGTTACAT AACTTACGGT AAATGGCCCG CCTGGCTGAC |
| 121 | CGCCCAACGA CCCCCGCCCA TTGACGTCAA TAATGACGTA TGTTCCCATA GTAACGCCAA |
| 181 | TAGGGACTTT CCATTGACGT CAATGGGTGG AGTATTTACG GTAAACTGCC CACTTGGCAG |
| 241 | TACATCAAGT GTATCATATG CCAAGTACGC CCCCTATTGA CGTCAATGAC GGTAAATGGC |
| 301 | CCGCCTGGCA TTATGCCCAG TACATGACCT TATGGGACTT TCCTACTTGG CAGTACATCT |
| 361 | ACGTATTAGT CATCGCTATT ACCATGGTCG AGGTGAGCCC CACGTTCTGC TTCACTCTCC |
| 421 | CCATCTCCCC CCCCTCCCCA CCCCCAATTT TGTATTTATT TATTTTTTAA TTATTTTGTG |
| 481 | CAGCGATGGG GGCGGGGGGG GGGGGGGGGC GCGCGCCAGG CGGGGCGGGG CGGGGCGAGG |
| 541 | GGCGGGGCGG GGCGAGGCGG AGAGGTGCGG CGGCAGCCAA TCAGAGCGGC GCGCTCCGAA |
| 601 | AGTTTCCTTT TATGGCGAGG CGGCGGCGGC GGCGGCCCTA TAAAAAGCGA AGCGCGCGGC |
| 661 | GGGCGGGAGT CGCTGCGCGC TGCCTTCGCC CCGTGCCCCG CTCCGCCGCC GCCTCGCGCC |
| 721 | GCCCGCCCCG GCTCTGACTG ACCGCGTTAC TCCCACAGGT GAGCGGGCGG GACGGCCCTT |
| 781 | CTCCTCCGGG CTGTAATTAG CGCTTGGTTT AATGACGGCT TGTTTCTTTT CTGTGGCTGC |
| 841 | GTGAAAGCCT TGAGGGGCTC CGGGAGGGCC CTTTGTGCGG GGGGAGCGGC TCGGGGGGTG |
| 901 | CGTGCGTGTG TGTGTGCGTG GGGAGCGCCG CGTGCGGCTC CGCGCTGCCC GGCGGCTGTG |
| 961 | AGCGCTGCGG GCGCGGCGCG GGGCTTTGTG CGCTCCGCAG TGTGCGCGAG GGGAGCGCGG |
| 1021 | CCGGGGGCGG TGCCCCGCGG TGCGGGGGGG GCTGCGAGGG GAACAAAGGC TGCGTGCGGG |
| 1081 | GTGTGTGCGT GGGGGGGTGA GCAGGGGGTG TGGGCGCGTC GGTCGGGCTG CAACCCCCCC |
| 1141 | TGCACCCCCC TCCCCGAGTT GCTGAGCACG GCCCGGCTTC GGGTGCGGGG CTCCGTACGG |
| 1201 | GGCGTGGCGC GGGGCTCGCC GTGCCGGGCG GGGGGTGGCG GCAGGTGGGG GTGCCGGGCG |
| 1261 | GGGCGGGGCC GCCTCGGGCC GGGGAGGGCT CGGGGGAGGG GCGCGGCGGC CCCCGGAGCG |
| 1321 | CCGGCGGCTG TCGAGGCGCG GCGAGCCGCA GCCATTGCCT TTTATGGTAA TCGTGCGAGA |
| 1381 | GGGCGCAGGG ACTTCCTTTG TCCCAAATCT GTGCGGAGCC GAAATCTGGG AGGCGCCGCC |
| 1441 | GCACCCCCTC TAGCGGGCGC GGGGCGAAGC GGTGCGGCGC CGGCAGGAAG GAAATGGGCG |
| 1501 | GGGAGGGCCT TCGTGCGTCG CCGCGCCGCC GTCCCCTTCT CCCTCTCCAG CCTCGGGGCT |
| 1561 | GTCCGCGGGG GGACGGCTGC CTTCGGGGGG GACGGGGCAG GGCGGGGTTC GGCTTCTGGC |
| 1621 | GTGTGACCGG CGGCTCTAGA GCCTCTGCTA ACCATGTTCA TGCCTTCTTC TTTTTCCTAC |
| 1681 | AGCTCCTGGG CAACGTGCTG GTTATTGTGC TGTCTCATCA TTTTGGCAAA |

### (SEQ ID NO: 22).

In some embodiments, the enhancer/promoter region comprises a ubiquitous promoter. In some embodiments, the enhancer/promoter region comprises a CMV promoter (SEQ ID NO: 23), an SV40 promoter (SEQ ID NO: 24), a PGK promoter (SEQ ID NO: 25), and/or a human beta-actin promoter (SEQ ID NO: 26). In some embodiments, the enhancer/promoter region comprises a polynucleotide that shares at least 75%, 80%, 85%, 90%, 95%, 96%, 97%, 98%, 99%, or 100% sequence identity with any one of SEQ ID NOs: 23-26:

| (SEQ ID NO: 23) | |
|---|---|
| 1 | GTGATGCGGT TTTGGCAGTA CATCAATGGG CGTGGATAGC GGTTTGACTC ACGGGGATTT |
| 61 | CCAAGTCTCC ACCCCATTGA CGTCAATGGG AGTTTGTTTT GGCACCAAAA TCAACGGGAC |
| 121 | TTTCCAAAAT GTCGTAACAA CTCCGCCCCA TTGACGCAAA TGGGCGGTAG GCGTGTACGG |
| 181 | TGGGAGGTCT ATATAAGCAG AGCT; |
| (SEQ ID NO: 24) | |
| 1 | GGTGTGGAAA GTCCCCAGGC TCCCCAGCAG GCAGAAGTAT GCAAAGCATG CATCTCAATT |
| 61 | AGTCAGCAAC CAGGTGTGGA AAGTCCCCAG GCTCCCCAGC AGGCAGAAGT ATGCAAAGCA |
| 121 | TGCATCTCAA TTAGTCAGCA ACCATAGTCC CGCCCCTAAC TCCGCCCATC CCGCCCCTAA |
| 181 | CTCCGCCCAG TTCCGCCCAT TCTCCGCCCC ATGGCTGACT AATTTTTTTT ATTTATGCAG |
| 241 | AGGCCGAGGC CGCCTCGGCC TCTGAGCTAT TCCAGAAGTA GTGAGGAGGC TTTTTTGGAG |
| 301 | GCCTAGGCTT TTGCAAA; |
| (SEQ ID NO: 25) | |
| 1 | GGGTAGGGGA GGCGCTTTTC CCAAGGCAGT CTGGAGCATG CGCTTTAGCA GCCCCGCTGG |
| 61 | GCACTTGGCG CTACACAAGT GGCCTCTGGC CTCGCACACA TTCCACATCC ACCGGTAGGC |
| 121 | GCCAACCGGC TCCGTTCTTT GGTGGCCCCT TCGCGCCACC TTCTACTCCT CCCCTAGTCA |
| 181 | GGAAGTTCCC CCCCGCCCCG CAGCTCGCGT CGTGCAGGAC GTGACAAATG GAAGTAGCAC |
| 241 | GTCTCACTAG TCTCGTGCAG ATGGACAGCA CCGCTGAGCA ATGGAAGCGG GTAGGCCTTT |
| 301 | GGGGCAGCGG CCAATAGCAG CTTTGCTCCT TCGCTTTCTG GGCTCAGAGG CTGGGAAGGG |
| 361 | GTGGGTCCGG GGGCGGGCTC AGGGGCGGGC TCAGGGGCGG GGCGGGCGCC CGAAGGTCCT |
| 421 | CCGGAGGCCC GGCATTCTGC ACGCTTCAAA AGCGCACGTC TGCCGCGCTG TTCTCCTCTT |
| 481 | CCTCATCTCC GGGCCTTTCG; |
| (SEQ ID NO: 26) | |
| 1 | CCTGCAGGGC CCACTAGTTC CATGTCCTTA TATGGACTCA TCTTTGCCTA TTGCGACACA |
| 61 | CACTCAATGA ACACCTACTA CGCGCTGCAA AGAGCCCCGC AGGCCTGAGG TGCCCCCACC |
| 121 | TCACCACTCT TCCTATTTTT GTGTAAAAAT CCAGCTTCTT GTCACCACCT CCAAGGAGGG |
| 181 | GGAGGAGGAG GAAGGCAGGT TCCTCTAGGC TGAGCCGAAT GCCCCTCTGT GGTCCCACGC |
| 241 | CACTGATCGC TGCATGCCCA CCACCTGGGT ACACACAGTC TGTGATTCCC GGAGCAGAAC |
| 301 | GGACCCTGCC CACCCGGTCT TGTGTGCTAC TCAGTGGACA GACCCAAGGC AAGAAAGGGT |
| 361 | GACAAGGACA GGGTCTTCCC AGGCTGGCTT TGAGTTCCTA GCACCGCCCC GCCCCCAATC |
| 421 | CTCTGTGGCA CATGGAGTCT TGGTCCCCAG AGTCCCCCAG CGGCCTCCAG ATGGTCTGGG |
| 481 | AGGGCAGTTC AGCTGTGGCT GCGCATAGCA GACATACAAC GGACGGTGGG CCCAGACCCA |
| 541 | GGCTGTGTAG ACCCAGCCCC CCCGCCCCGC AGTGCCTAGG TCACCCACTA ACGCCCCAGG |
| 601 | CCTGGTCTTG GCTGGGCGTG ACTGTTACCC TCAAAAGCAG GCAGCTCCAG GGTAAAAGGT |
| 661 | GCCCTGCCCT GTAGAGCCCA CCTTCCTTCC CAGGGCTGCG GCTGGGTAGG TTTGTAGCCT |
| 721 | TCATCACGGG CCACCTCCAG CCACTGGACC GCTGGCCCCT GCCCTGTCCT GGGGAGTGTG |
| 781 | GTCCTGCGAC TTCTAAGTGG CCGCAAGCCA CCTGACTCCC CCAACACCAC ACTCTACCTC |
| 841 | TCAAGCCCAG GTCTCTCCCT AGTGACCCAC CCAGCACATT TAGCTAGCTG AGCCCCACAG |
| 901 | CCAGAGGTCC TCAGGCCCTG CTTTCAGGGC AGTTGCTCTG AAGTCGGCAA GGGGGAGTGA |
| 961 | CTGCCTGGCC ACTCCATGCC CTCCAAGAGC TCCTTCTGCA GGAGCGTACA GAACCCAGGG |
| 1021 | CCCTGGCACC CGTGCAGACC CTGGCCCACC CCACCTGGGC GCTCAGTGCC CAAGAGATGT |
| 1081 | CCACACCTAG GATGTCCCGC GGTGGGTGGG GGGCCCGAGA GACGGGCAGG CCGGGGGCAG |
| 1141 | GCCTGGCCAT GCGGGGCCGA ACCGGGCACT GCCCAGCGTG GGGCGCGGGG GCCACGGCGC |
| 1201 | GCGCCCCCAG CCCCCGGGCC CAGCACCCCA AGGCGGCCAA CGCCAAAACT CTCCCTCCTC |
| 1261 | CTCTTCCTCA ATCTCGCTCT CGCTCTTTTT TTTTTTCGCA AAAGGAGGGG AGAGGGGGTA |
| 1321 | AAAAAATGCT GCACTGTGCG GCGAAGCCGG TGAGTGAGCG GCGCGGGGCC AATCAGCGTG |
| 1381 | CGCCGTTCCG AAAGTTGCCT TTTATGGCTC GAGCGGCCGC GGCGGCGCCC TATAAAACCC |
| 1441 | AGCGGCGCGA CGCGCCACCA CCGCCGAGAC CGCGTCCGCC CCGCGAGCAC AGAGCCTCGC |
| 1501 | CTTTGCCGAT CCGCCGCCCG TCCACACCCG CCGCCAGGTA AGCCCGGCCA GCCGACCGGG |
| 1561 | GCAGGCGGCT CACGGCCCGG CCGCAGGCGG CCGCGGCCCC TTCGCCCGTG CAGAGCCGCC |
| 1621 | GTCTGGGCCG CAGCGGGGGG CGCATGGGGG GGGAACCGGA CCGCCGTGGG GGGCGCGGGA |
| 1681 | GAAGCCCCTG GGCCTCCGGA GATGGGGGAC ACCCCACGCC AGTTCGGAGG CGCGAGGCCG |
| 1741 | CGCTCGGGAG GCGCGCTCCG GGGGTGCCGC TCTCGGGGCG GGGGCAACCG GCGGGGTCTT |
| 1801 | TGTCTGAGCC GGGCTCTTGC CAATGGGGAT CGCAGGGTGG GCGCGGCGGA GCCCCCGCCA |
| 1861 | GGCCCGGTGG GGGCTGGGGC GCCATTGCGC GTGCGCGCTG GTCCTTTGGG CGCTAACTGC |
| 1921 | GTGCGCGCTG GGAATTGGCG CTAATTGCGC GTGCGCGCTG GGACTCAAGG CGCTAACTGC |
| 1981 | GCGTGCGTTC TGGGGCCCGG GGTGCCGCGG CCTGGGCTGG GGCGAAGGCG GGCTCGGCCG |
| 2041 | GAAGGGGTGG GGTCGCCGCG GCTCCCGGGC GCTTGCGCGC ACTTCCTGCC CGAGCCGCTG |
| 2101 | GCCGCCCGAG GGTGTGGCCG CTGCGTGCGC GCGCGCCGAC CCGGCGCTGT TTGAACCGGG |
| 2161 | CGGAGGCGGG GCTGGCGCCC GGTTGGGAGG GGGTTGGGGC CTGGCTTCCT GCCGCGCGCC |
| 2221 | GCGGGGACGC CTCCGACCAG TGTTTGCCTT TTATGGTAAT AACGCGGCCG GCCCGGCTTC |
| 2281 | CTTTGTCCCC AATCTGGGCG CGCGCCGGCG CCCCCTGGCG GCCTAAGGAC TCGGCGCGCC |
| 2341 | GGAAGTGGCC AGGGCGGGGG CGACCTCGGC TCACAGCGCG CCCGGCTATT CTCGCAGCTC |
| 2401 | ACC. |

Further exemplary promoters include, but are not limited to, human Elongation Factor 1 alpha promoter (EFS), SV40 early promoter, mouse mammary tumor virus long terminal repeat (LTR) promoter; adenovirus major late promoter (Ad MLP); a herpes simplex virus (HSV) promoter, an endogenous cellular promoter that is heterologous to the gene of interest, a cytomegalovirus (CMV) promoter such as the CMV immediate early promoter region (CMVIE), a Rous sarcoma virus (RSV) promoter, synthetic promoters, hybrid promoters, and the like.

In some embodiments, the 3′ UTR comprises a polynucleotide (WPRE element) that shares at least 75%, 80%, 85%, 90%, 95%, 96%, 97%, 98%, 99%, or 100% sequence identity with SEQ ID NO: 27:

| (SEQ ID NO: 27) | |
|---|---|
| 1 | ATTCGAGCAT CTTACCGCCA TTTATTCCCA TATTTGTTCT GTTTTTCTTG ATTTGGGTAT |
| 61 | ACATTTAAAT GTTAATAAAA CAAAATGGTG GGGCAATCAT TTACATTTTT AGGGATATGT |
| 121 | AATTACTAGT TCAGGTGTAT TGCCACAAGA CAAACATGTT AAGAAACTTT CCCGTTATTT |
| 181 | ACGCTCTGTT CCTGTTAATC AACCTCTGGA TTACAAAATT TGTGAAAGAT TGACTGATAT |
| 241 | TCTTAACTAT GTTGCTCCTT TTACGCTGTG TGGATATGCT GCTTTAATGC CTCTGTATCA |
| 301 | TGCTATTGCT TCCCGTACGG CTTTCGTTTT CTCCTCCTTG TATAAATCCT GGTTGCTGTC |
| 361 | TCTTTATGAG GAGTTGTGGC CCGTTGTCCG TCAACGTGGC GTGGTGTGCT CTGTGTTTGC |
| 421 | TGACGCAACC CCCACTGGCT GGGGCATTGC CACCACCTGT CAACTCCTTT CTGGGACTTT |
| 481 | CGCTTTCCCC CTCCCGATCG CCACGGCAGA ACTCATCGCC GCCTGCCTTG CCCGCTGCTG |
| 541 | GACAGGGGCT AGGTTGCTGG GCACTGATAA TTCCGTGGTG TTGTCGGGGA AGGGCC. |

In some embodiment, the expression cassette shares at least 95% identity to a sequence selected from SEQ ID NOs: 8-10. In an embodiment, the expression cassette shares complete identity to a sequence selected from SEQ ID NOs: 8-10, or shares at least 80%, at least 85%, at least 90%, at least 95%, at least 98%, or at least 99% identity to a sequence selected from SEQ ID NOs: 8-10:

| (SEQ ID NO: 8) | |
|---|---|
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT TGTAGTTAAT GATTAACCCG CCATGCTACT TATCTACCAG GGTAATGGGG |
| 181 | ATCCTCTAGA ACTATAGCTA GTCGACATTG ATTATTGACT AGTTATTAAT AGTAATCAAT |
| 241 | TACGGGGTCA TTAGTTCATA GCCCATATAT GGAGTTCCGC GTTACATAAC TTACGGTAAA |
| 301 | TGGCCCGCCT GGCTGACCGC CCAACGACCC CCGCCCATTG ACGTCAATAA TGACGTATGT |
| 361 | TCCCATAGTA ACGCCAATAG GGACTTTCCA TTGACGTCAA TGGGTGGAGT ATTTACGGTA |
| 421 | AACTGCCCAC TTGGCAGTAC ATCAAGTGTA TCATATGCCA AGTACGCCCC CTATTGACGT |
| 481 | CAATGACGGT AAATGGCCCG CCTGGCATTA TGCCCAGTAC ATGACCTTAT GGGACTTTCC |
| 541 | TACTTGGCAG TACATCTACG TATTAGTCAT CGCTATTACC ATGGTCGAGG TGAGCCCCAC |
| 601 | GTTCTGCTTC ACTCTCCCCA TCTCCCCCCC CTCCCCACCC CCAATTTTGT ATTTATTTAT |
| 661 | TTTTTAATTA TTTTGTGCAG CGATGGGGGC GGGGGGGGGG GGGGGGCGCG CGCCAGGCGG |
| 721 | GGCGGGGCGG GGCGAGGGGC GGGGCGGGGC GAGGCGGAGA GGTGCGGCGG CAGCCAATCA |
| 781 | GAGCGGCGCG CTCCGAAAGT TTCCTTTTAT GGCGAGGCGG CGGCGGCGGC GGCCCTATAA |
| 841 | AAAGCGAAGC GCGCGGCGGG CGGGAGTCGC TGCGCGCTGC CTTCGCCCCG TGCCCCGCTC |
| 901 | CGCCGCCGCC TCGCGCCGCC CGCCCCGGCT CTGACTGACC GCGTTACTCC CACAGGTGAG |
| 961 | CGGGCGGGAC GGCCCTTCTC CTCCGGGCTG TAATTAGCGC TTGGTTTAAT GACGGCTTGT |
| 1021 | TTCTTTTCTG TGGCTGCGTG AAAGCCTTGA GGGGCTCCGG GAGGGCCCTT TGTGCGGGGG |
| 1081 | GAGCGGCTCG GGGGGTGCGT GCGTGTGTGT GTGCGTGGGG AGCGCCGCGT GCGGCTCCGC |
| 1141 | GCTGCCCGGC GGCTGTGAGC GCTGCGGGCG CGGCGCGGGG CTTTGTGCGC TCCGCAGTGT |
| 1201 | GCGCGAGGGG AGCGCGGCCG GGGGCGGTGC CCCGCGGTGC GGGGGGGGCT GCGAGGGGAA |
| 1261 | CAAAGGCTGC GTGCGGGGTG TGTGCGTGGG GGGGTGAGCA GGGGGTGTGG GCGCGTCGGT |
| 1321 | CGGGCTGCAA CCCCCCCTGC ACCCCCCTCC CCGAGTTGCT GAGCACGGCC CGGCTTCGGG |
| 1381 | TGCGGGGCTC CGTACGGGGC GTGGCGCGGG GCTCGCCGTG CCGGGCGGGG GGTGGCGGCA |
| 1441 | GGTGGGGGTG CCGGGCGGGG CGGGGCCGCC TCGGGCCGGG GAGGGCTCGG GGGAGGGGCG |
| 1501 | CGGCGGCCCC CGGAGCGCCG GCGGCTGTCG AGGCGCGGCG AGCCGCAGCC ATTGCCTTTT |
| 1561 | ATGGTAATCG TGCGAGAGGG CGCAGGGACT TCCTTTGTCC CAAATCTGTG CGGAGCCGAA |
| 1621 | ATCTGGGAGG CGCCGCCGCA CCCCCTCTAG CGGGCGCGGG GCGAAGCGGT GCGGCGCCGG |
| 1681 | CAGGAAGGAA ATGGGCGGGG AGGGCCTTCG TGCGTCGCCG CGCCGCCGTC CCCTTCTCCC |
| 1741 | TCTCCAGCCT CGGGGCTGTC CGCGGGGGGA CGGCTGCCTT CGGGGGGGAC GGGGCAGGGC |
| 1801 | GGGGTTCGGC TTCTGGCGTG TGACCGGCGG CTCTAGAGCC TCTGCTAACC ATGTTCATGC |
| 1861 | CTTCTTCTTT TTCCTACAGC TCCTGGGCAA CGTGCTGGTT ATTGTGCTGT CTCATCATTT |
| 1921 | TGGCAAAGAA TTCGAGCGGC CGCCAGCCGC CACCATGGTC TGCTTCAGAC TGTTCCCTGT |
| 1981 | CCCTGGATCT GGTCTGGTGC TTGTGTGCTT GGTGCTGGGT GCTGTGAGAT CCTATGCCCT |
| 2041 | TGAGCTGAAC CTGACTGACT CAGAAAATGC CACTTGCCTG TATGCCAAGT GGCAGATGAA |
| 2101 | CTTCACTGTG AGATATGAGA CTACCAACAA GACCTACAAG ACTGTGACCA TCTCAGACCA |
| 2161 | TGGCACTGTC ACCTACAATG GATCAATCTG TGGTGATGAT CAGAATGGCC CAAAGATAGC |
| 2221 | AGTGCAGTTT GGGCCCGGTT TTTCCTGGAT TGCTAACTTC ACCAAGGCAG CCTCCACCTA |
| 2281 | CAGCATTGAC TCAGTCAGCT TCAGCTACAA CACTGGGGAT AACACCACCT TCCCTGACGC |
| 2341 | AGAGGACAAG GGAATCCTTA CTGTGGACGA ACTCCTGGCA ATCAGAATCC CCCTTAACGA |
| 2401 | CCTGTTCAGA TGCAACTCCC TTTCAACCCT TGAAAAGAAT GATGTGGTGC AACACTATTG |
| 2461 | GGACGTCCTG GTGCAAGCCT TTGTGCAGAA TGGGACAGTG AGTACCAACG AGTTCCTCTG |
| 2521 | TGACAAGGAC AAGACCAGCA CTGTGGCCCC CACTATCCAC ACCACTGTGC CCAGCCCTAC |
| 2581 | CACTACCCCC ACCCCTAAAG AGAAGCCAGA AGCTGGAACC TACTCAGTCA ACAATGGAAA |
| 2641 | TGACACATGC CTCCTTGCCA CCATGGGACT GCAGCTGAAC ATCACTCAGG ACAAGGTGGC |
| 2701 | CTCAGTGATT AACATCAACC CTAACACCAC TCATAGCACT GGGAGCTGCA GATCACATAC |
| 2761 | AGCTCTGCTG AGGCTCAACT CCTCCACCAT CAAGTACCTG GACTTTGTGT TTGCTGTGAA |
| 2821 | GAATGAGAAC AGGTTCTACC TCAAGGAAGT GAACATTTCC ATGTACCTGG TCAATGGTTC |
| 2881 | AGTGTTCTCT ATTGCCAACA ACAATCTGAG CTACTGGGAT GCACCCCTGG GATCCTCCTA |
| 2941 | CATGTGCAAC AAGGAGCAGA CTGTGAGTGT GTCAGGTGCT TTTCAGATCA ACACTTTTGA |
| 3001 | CCTGAGGGTG CAGCCCTTCA ATGTGACTCA GGGAAAGTAC TCCACTGCAC AAGAGTGTTC |
| 3061 | CTTGGATGAT GACACTATCC TCATCCCCAT TATTGTGGGA GCTGGACTGT CAGGATTGAT |
| 3121 | TATAGTGATT GTGATTGCTT ATGTGATTGG AAGGAGAAAG AGCTATGCTG GCTACCAGAC |
| 3181 | CCTGTAAAAG GGCGAATTCC AGCACACGCG TCCTAGGAGC TCGAGTACTA CTGGCGGCCG |
| 3241 | TTACTAGTGG ATCCGCGGTA CAAGTAAGCA TGCAAGCTTC GAGGACGGGG TGAACTACGC |
| 3301 | CTGAATCAAG CTTATCGATA AATTCGAGCA TCTTACCGCC ATTTATTCCC ATATTTGTTC |
| 3361 | TGTTTTTCTT GATTTGGGTA TACATTTAAA TGTTAATAAA ACAAAATGGT GGGGCAATCA |
| 3421 | TTTACATTTT TAGGGATATG TAATTACTAG TTCAGGTGTA TTGCCACAAG ACAAACATGT |
| 3481 | TAAGAAACTT TCCCGTTATT TACGCTCTGT TCCTGTTAAT CAACCTCTGG ATTACAAAAT |
| 3541 | TTGTGAAAGA TTGACTGATA TTCTTAACTA TGTTGCTCCT TTTACGCTGT GTGGATATGC |
| 3601 | TGCTTTAATG CCTCTGTATC ATGCTATTGC TTCCCGTACG GCTTTCGTTT TCTCCTCCTT |
| 3661 | GTATAAATCC TGGTTGCTGT CTCTTTATGA GGAGTTGTGG CCCGTTGTCC GTCAACGTGG |
| 3721 | CGTGGTGTGC TCTGTGTTTG CTGACGCAAC CCCCACTGGC TGGGGCATTG CCACCACCTG |
| 3781 | TCAACTCCTT TCTGGGACTT TCGCTTTCCC CCTCCCGATC GCCACGGCAG AACTCATCGC |
| 3841 | CGCCTGCCTT GCCCGCTGCT GGACAGGGGC TAGGTTGCTG GGCACTGATA ATTCCGTGGT |
| 3901 | GTTGTCGGGG AAGGGCCTCG ATACCGTCGA TATCGATCCT GGCTAATAAA GGAAATTTAT |
| 3961 | TTTCATTGCA ATAGTGTGTT GGAATTTTTT GTGTCTCTCA CTCGGAAGGA CATATGGGAG |
| 4021 | GGCAAATCAT TTAAAACATC AGAATGAGTA TTTGGTTTAG AGTTTGGCAA CATATGCCCA |
| 4081 | TATGCTGGCT GCCATGAACA AAGGTTGGCT ATAAAGAGGT CATCAGTATA TGAAACAGCC |
| 4141 | CCCTGCTGTC CATTCCTTAT TCCATAGAAA AGCCTTGACT TGAGGTTAGA TTTTTTTTAT |
| 4201 | ATTTTGTTTT GTGTTATTTT TTTCTTTAAC ATCCCTAAAA TTTTCCTTAC ATGTTTTACT |
| 4261 | AGCCAGATTT TTCCTCCTCT CCTGACTACT CCCAGTCATA GCTGTCCCTC TTCTCTTATG |
| 4321 | GAGATCGAAG CAATTCGTTG ATCTGAATTT CGACCACCCA TAATAGATCT CCCATTACCC |
| 4381 | TGGTAGATAA GTAGCATGGC GGGTTAATCA TTAACTACAA GGAACCCCTA GTGATGGAGT |
| 4441 | TGGCCACTCC CTCTCTGCGC GCTCGCTCGC TCACTGAGGC CGGGCGACCA AAGGTCGCCC |
| 4501 | GACGCCCGGG CTTTGCCCGG GCGGCCTCAG TGAGCGAGCG AGCGCGCAG; |
| (SEQ ID NO: 9) | |
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT TGTAGTTAAT GATTAACCCG CCATGCTACT TATCTACCAG GGTAATGGGG |
| 181 | ATCCTCTAGA ACTATAGCTA GTCGACATTG ATTATTGACT AGTTATTAAT AGTAATCAAT |
| 241 | TACGGGGTCA TTAGTTCATA GCCCATATAT GGAGTTCCGC GTTACATAAC TTACGGTAAA |
| 301 | TGGCCCGCCT GGCTGACCGC CCAACGACCC CCGCCCATTG ACGTCAATAA TGACGTATGT |
| 361 | TCCCATAGTA ACGCCAATAG GGACTTTCCA TTGACGTCAA TGGGTGGAGT ATTTACGGTA |
| 421 | AACTGCCCAC TTGGCAGTAC ATCAAGTGTA TCATATGCCA AGTACGCCCC CTATTGACGT |
| 481 | CAATGACGGT AAATGGCCCG CCTGGCATTA TGCCCAGTAC ATGACCTTAT GGGACTTTCC |
| 541 | TACTTGGCAG TACATCTACG TATTAGTCAT CGCTATTACC ATGGTCGAGG TGAGCCCCAC |
| 601 | GTTCTGCTTC ACTCTCCCCA TCTCCCCCCC CTCCCCACCC CCAATTTTGT ATTTATTTAT |
| 661 | TTTTTAATTA TTTTGTGCAG CGATGGGGGC GGGGGGGGGG GGGGGGCGCG CGCCAGGCGG |
| 721 | GGCGGGGCGG GGCGAGGGGC GGGGCGGGGC GAGGCGGAGA GGTGCGGCGG CAGCCAATCA |
| 781 | GAGCGGCGCG CTCCGAAAGT TTCCTTTTAT GGCGAGGCGG CGGCGGCGGC GGCCCTATAA |
| 841 | AAAGCGAAGC GCGCGGCGGG CGGGAGTCGC TGCGCGCTGC CTTCGCCCCG TGCCCCGCTC |
| 901 | CGCCGCCGCC TCGCGCCGCC CGCCCCGGCT CTGACTGACC GCGTTACTCC CACAGGTGAG |
| 961 | CGGGCGGGAC GGCCCTTCTC CTCCGGGCTG TAATTAGCGC TTGGTTTAAT GACGGCTTGT |
| 1021 | TTCTTTTCTG TGGCTGCGTG AAAGCCTTGA GGGGCTCCGG GAGGGCCCTT TGTGCGGGGG |
| 1081 | GAGCGGCTCG GGGGGTGCGT GCGTGTGTGT GTGCGTGGGG AGCGCCGCGT GCGGCTCCGC |
| 1141 | GCTGCCCGGC GGCTGTGAGC GCTGCGGGCG CGGCGCGGGG CTTTGTGCGC TCCGCAGTGT |
| 1201 | GCGCGAGGGG AGCGCGGCCG GGGGCGGTGC CCCGCGGTGC GGGGGGGGCT GCGAGGGGAA |
| 1261 | CAAAGGCTGC GTGCGGGGTG TGTGCGTGGG GGGGTGAGCA GGGGGTGTGG GCGCGTCGGT |
| 1321 | CGGGCTGCAA CCCCCCCTGC ACCCCCCTCC CCGAGTTGCT GAGCACGGCC CGGCTTCGGG |
| 1381 | TGCGGGGCTC CGTACGGGGC GTGGCGCGGG GCTCGCCGTG CCGGGCGGGG GGTGGCGGCA |
| 1441 | GGTGGGGGTG CCGGGGGGGG CGGGGCCGCC TCGGGCCGGG GAGGGCTCGG GGGAGGGGCG |
| 1501 | CGGCGGCCCC CGGAGCGCCG GCGGCTGTCG AGGCGCGGCG AGCCGCAGCC ATTGCCTTTT |
| 1561 | ATGGTAATCG TGCGAGAGGG CGCAGGGACT TCCTTTGTCC CAAATCTGTG CGGAGCCGAA |
| 1621 | ATCTGGGAGG CGCCGCCGCA CCCCCTCTAG CGGGCGCGGG GCGAAGCGGT GCGGCGCCGG |
| 1681 | CAGGAAGGAA ATGGGGGGGG AGGGCCTTCG TGCGTCGCCG CGCCGCCGTC CCCTTCTCCC |
| 1741 | TCTCCAGCCT CGGGGCTGTC CGCGGGGGGA CGGCTGCCTT CGGGGGGGAC GGGGCAGGGC |
| 1801 | GGGGTTCGGC TTCTGGCGTG TGACCGGCGG CTCTAGAGCC TCTGCTAACC ATGTTCATGC |
| 1861 | CTTCTTCTTT TTCCTACAGC TCCTGGGCAA CGTGCTGGTT ATTGTGCTGT CTCATCATTT |
| 1921 | TGGCAAAGAA TTCGAGCGGC CGCCAGCCGC CACCATGGTG TGCTTTAGAC TGTTTCCTGT |
| 1981 | GCCTGGTTCA GGGCTGGTCC TGGTCTGTCT GGTGCTGGGG GCTGTCAGAA GCTATGCCTT |
| 2041 | GGAGCTGAAC CTCACTGATA GTGAAAATGC CACTTGTCTG TATGCTAAGT GGCAGATGAA |
| 2101 | CTTCACTGTG AGATATGAAA CCACCAACAA GACTTACAAA ACAGTGACCA TCTCAGATCA |
| 2161 | TGGAACTGTG ACCTACAACG GCAGCATTTG TGGAGACGAC CAGAACGGAC CAAAAATCGC |
| 2221 | TGTCCAATTT GGGCCTGGAT TCTCCTGGAT TGCCAATTTC ACTAAAGCTG CCTCCACATA |
| 2281 | TTCAATTGAC TCAGTGTCCT TCTCCTACAA CACTGGGGAC AACACTACTT TCCCTGATGC |
| 2341 | TGAAGATAAG GGAATCTTGA CAGTGGATGA GCTGCTGGCT ATCAGGATCC CTTTGAATGA |
| 2401 | CCTGTTTAGG TGTAATTCAC TGAGCACTCT GGAGAAGAAC GACGTGGTGC AGCACTACTG |
| 2461 | GGACGTGCTG GTGCAGGCCT TTGTGCAGAA CGGCACTGTG TCCACCAACG AATTCCTGTG |
| 2521 | TGATAAGGAC AAAACTTCCA CTGTGGCACC TACAATTCAC ACTACTGTGC CTTCACCTAC |
| 2581 | CACCACTCCA ACTCCAAAGG AAAAGCCTGA AGCAGGAACC TACTCTGTGA ACAATGGCAA |
| 2641 | TGATACCTGT CTGTTGGCCA CCATGGGCCT CCAACTGAAC ATTACTCAGG ACAAGGTGGC |
| 2701 | CTCAGTGATT AACATTAACC CCAACACTAC CCACTCCACT GGCAGCTGTA GATCACACAC |
| 2761 | AGCCTTGCTC AGACTGAATA GCAGCACCAT CAAGTATTTG GATTTTGTGT TTGCAGTGAA |
| 2821 | GAATGAAAAC AGGTTCTACC TGAAGGAAGT CAACATCTCA ATGTACCTGG TGAACGGCTC |
| 2881 | AGTGTTCAGC ATTGCCAACA ACAACCTCTC CTATTGGGAC GCTCCACTGG GGAGCAGCTA |
| 2941 | CATGTGTAAC AAGGAACAGA CTGTGTCAGT GTCAGGAGCC TTCCAGATTA ACACCTTTGA |
| 3001 | TCTGAGGGTC CAACCCTTTA ATGTCACTCA AGGAAAGTAT AGCACTGCCC AGGAGTGCTC |
| 3061 | CCTGGATGAT GACACCATTC TGATTCCAAT CATTGTGGGT GCAGGACTTT CTGGGCTTAT |
| 3121 | TATTGTGATT GTGATTGCCT ATGTGATTGG CAGAAGGAAA TCCTATGCAG GGTACCAAAC |
| 3181 | TCTGTAAAAG GGCGAATTCC AGCACACGCG TCCTAGGAGC TCGAGTACTA CTGGCGGCCG |
| 3241 | TTACTAGTGG ATCCGCGGTA CAAGTAAGCA TGCAAGCTTC GAGGACGGGG TGAACTACGC |
| 3301 | CTGAATCAAG CTTATCGATA AATTCGAGCA TCTTACCGCC ATTTATTCCC ATATTTGTTC |
| 3361 | TGTTTTTCTT GATTTGGGTA TACATTTAAA TGTTAATAAA ACAAAATGGT GGGGCAATCA |
| 3421 | TTTACATTTT TAGGGATATG TAATTACTAG TTCAGGTGTA TTGCCACAAG ACAAACATGT |
| 3481 | TAAGAAACTT TCCCGTTATT TACGCTCTGT TCCTGTTAAT CAACCTCTGG ATTACAAAAT |
| 3541 | TTGTGAAAGA TTGACTGATA TTCTTAACTA TGTTGCTCCT TTTACGCTGT GTGGATATGC |
| 3601 | TGCTTTAATG CCTCTGTATC ATGCTATTGC TTCCCGTACG GCTTTCGTTT TCTCCTCCTT |
| 3661 | GTATAAATCC TGGTTGCTGT CTCTTTATGA GGAGTTGTGG CCCGTTGTCC GTCAACGTGG |
| 3721 | CGTGGTGTGC TCTGTGTTTG CTGACGCAAC CCCCACTGGC TGGGGCATTG CCACCACCTG |
| 3781 | TCAACTCCTT TCTGGGACTT TCGCTTTCCC CCTCCCGATC GCCACGGCAG AACTCATCGC |
| 3841 | CGCCTGCCTT GCCCGCTGCT GGACAGGGGC TAGGTTGCTG GGCACTGATA ATTCCGTGGT |
| 3901 | GTTGTCGGGG AAGGGCCTCG ATACCGTCGA TATCGATCCT GGCTAATAAA GGAAATTTAT |
| 3961 | TTTCATTGCA ATAGTGTGTT GGAATTTTTT GTGTCTCTCA CTCGGAAGGA CATATGGGAG |
| 4021 | GGCAAATCAT TTAAAACATC AGAATGAGTA TTTGGTTTAG AGTTTGGCAA CATATGCCCA |
| 4081 | TATGCTGGCT GCCATGAACA AAGGTTGGCT ATAAAGAGGT CATCAGTATA TGAAACAGCC |
| 4141 | CCCTGCTGTC CATTCCTTAT TCCATAGAAA AGCCTTGACT TGAGGTTAGA TTTTTTTTAT |
| 4201 | ATTTTGTTTT GTGTTATTTT TTTCTTTAAC ATCCCTAAAA TTTTCCTTAC ATGTTTTACT |
| 4261 | AGCCAGATTT TTCCTCCTCT CCTGACTACT CCCAGTCATA GCTGTCCCTC TTCTCTTATG |
| 4321 | GAGATCGAAG CAATTCGTTG ATCTGAATTT CGACCACCCA TAATAGATCT CCCATTACCC |
| 4381 | TGGTAGATAA GTAGCATGGC GGGTTAATCA TTAACTACAA GGAACCCCTA GTGATGGAGT |
| 4441 | TGGCCACTCC CTCTCTGCGC GCTCGCTCGC TCACTGAGGC CGGGCGACCA AAGGTCGCCC |
| 4501 | GACGCCCGGG CTTTGCCCGG GCGGCCTCAG TGAGCGAGCG AGCGCGCAG; |
| and | |
| (SEQ ID NO: 10) | |
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT TGTAGTTAAT GATTAACCCG CCATGCTACT TATCTACCAG GGTAATGGGG |
| 181 | ATCCTCTAGA ACTATAGCTA GTCGACATTG ATTATTGACT AGTTATTAAT AGTAATCAAT |
| 241 | TACGGGGTCA TTAGTTCATA GCCCATATAT GGAGTTCCGC GTTACATAAC TTACGGTAAA |
| 301 | TGGCCCGCCT GGCTGACCGC CCAACGACCC CCGCCCATTG ACGTCAATAA TGACGTATGT |
| 361 | TCCCATAGTA ACGCCAATAG GGACTTTCCA TTGACGTCAA TGGGTGGAGT ATTTACGGTA |
| 421 | AACTGCCCAC TTGGCAGTAC ATCAAGTGTA TCATATGCCA AGTACGCCCC CTATTGACGT |
| 481 | CAATGACGGT AAATGGCCCG CCTGGCATTA TGCCCAGTAC ATGACCTTAT GGGACTTTCC |
| 541 | TACTTGGCAG TACATCTACG TATTAGTCAT CGCTATTACC ATGGTCGAGG TGAGCCCCAC |
| 601 | GTTCTGCTTC ACTCTCCCCA TCTCCCCCCC CTCCCCACCC CCAATTTTGT ATTTATTTAT |
| 661 | TTTTTAATTA TTTTGTGCAG CGATGGGGGC GGGGGGGGGG GGGGGGCGCG CGCCAGGCGG |
| 721 | GGCGGGGCGG GGCGAGGGGC GGGGCGGGGC GAGGCGGAGA GGTGCGGCGG CAGCCAATCA |
| 781 | GAGCGGCGCG CTCCGAAAGT TTCCTTTTAT GGCGAGGCGG CGGCGGCGGC GGCCCTATAA |
| 841 | AAAGCGAAGC GCGCGGCGGG CGGGAGTCGC TGCGCGCTGC CTTCGCCCCG TGCCCCGCTC |
| 901 | CGCCGCCGCC TCGCGCCGCC CGCCCCGGCT CTGACTGACC GCGTTACTCC CACAGGTGAG |
| 961 | CGGGCGGGAC GGCCCTTCTC CTCCGGGCTG TAATTAGCGC TTGGTTTAAT GACGGCTTGT |
| 1021 | TTCTTTTCTG TGGCTGCGTG AAAGCCTTGA GGGGCTCCGG GAGGGCCCTT TGTGCGGGGG |
| 1081 | GAGCGGCTCG GGGGGTGCGT GCGTGTGTGT GTGCGTGGGG AGCGCCGCGT GCGGCTCCGC |
| 1141 | GCTGCCCGGC GGCTGTGAGC GCTGCGGGCG CGGCGCGGGG CTTTGTGCGC TCCGCAGTGT |
| 1201 | GCGCGAGGGG AGCGCGGCCG GGGGCGGTGC CCCGCGGTGC GGGGGGGGCT GCGAGGGGAA |
| 1261 | CAAAGGCTGC GTGCGGGGTG TGTGCGTGGG GGGGTGAGCA GGGGGTGTGG GCGCGTCGGT |
| 1321 | CGGGCTGCAA CCCCCCCTGC ACCCCCCTCC CCGAGTTGCT GAGCACGGCC CGGCTTCGGG |
| 1381 | TGCGGGGCTC CGTACGGGGC GTGGCGCGGG GCTCGCCGTG CCGGGGGGGG GGTGGCGGCA |
| 1441 | GGTGGGGGTG CCGGGGGGGG CGGGGCCGCC TCGGGCCGGG GAGGGCTCGG GGGAGGGGCG |
| 1501 | CGGCGGCCCC CGGAGCGCCG GCGGCTGTCG AGGCGCGGCG AGCCGCAGCC ATTGCCTTTT |
| 1561 | ATGGTAATCG TGCGAGAGGG CGCAGGGACT TCCTTTGTCC CAAATCTGTG CGGAGCCGAA |
| 1621 | ATCTGGGAGG CGCCGCCGCA CCCCCTCTAG CGGGCGCGGG GCGAAGCGGT GCGGCGCCGG |
| 1681 | CAGGAAGGAA ATGGGCGGGG AGGGCCTTCG TGCGTCGCCG CGCCGCCGTC CCCTTCTCCC |
| 1741 | TCTCCAGCCT CGGGGCTGTC CGCGGGGGGA CGGCTGCCTT CGGGGGGGAC GGGGCAGGGC |
| 1801 | GGGGTTCGGC TTCTGGCGTG TGACCGGCGG CTCTAGAGCC TCTGCTAACC ATGTTCATGC |
| 1861 | CTTCTTCTTT TTCCTACAGC TCCTGGGCAA CGTGCTGGTT ATTGTGCTGT CTCATCATTT |
| 1921 | TGGCAAAGAA TTCGAGCGGC CGCCAGCCGC CACCATGGTC TGTTTTAGGC TGTTCCCTGT |
| 1981 | CCCTGGTTCA GGACTGGTCT TAGTGTGTCT GGTGCTTGGA GCTGTCAGAA GCTATGCCCT |
| 2041 | GGAGCTGAAC CTGACTGACT CAGAAAATGC CACTTGCCTG TATGCCAAGT GGCAGATGAA |
| 2101 | CTTCACTGTC AGATATGAAA CCACCAACAA GACCTATAAG ACTGTGACCA TCTCAGACCA |
| 2161 | TGGCACTGTG ACTTACAATG GGTCAATTTG TGGAGATGAC CAGAATGGCC CTAAGATAGC |
| 2221 | TGTCCAGTTT GGTCCAGGAT TCAGCTGGAT TGCCAACTTC ACCAAGGCAG CCAGCACCTA |
| 2281 | CAGCATTGAC TCTGTGTCCT TCTCCTACAA CACAGGAGAC AACACCACTT TCCCTGATGC |
| 2341 | AGAGGACAAA GGTATCCTGA CTGTGGATGA GTTGCTGGCA ATCAGGATCC CACTGAACGA |
| 2401 | TCTGTTCAGG TGCAACTCAC TGTCCACTCT GGAAAAGAAT GATGTGGTGC AGCACTATTG |
| 2461 | GGATGTGCTA GTCCAGGCCT TTGTCCAGAA TGGGACTGTG TCAACTAATG AGTTCCTGTG |
| 2521 | TGACAAGGAC AAGACAAGCA CTGTAGCCCC CACTATCCAT ACCACAGTAC CTAGCCCCAC |
| 2581 | CACTACTCCA ACCCCCAAGG AGAAGCCTGA GGCTGGCACC TACTCAGTGA ACAATGGGAA |
| 2641 | TGACACCTGT TTGCTGGCCA CTATGGGACT CCAACTGAAC ATCACCCAGG ACAAAGTGGC |
| 2701 | CTCTGTGATC AATATCAATC CCAACACCAC CCACAGCACT GGGTCCTGCA GAAGCCACAC |
| 2761 | TGCCCTCCTG AGGCTCAACT CATCAACTAT CAAGTACTTG GATTTTGTGT TTGCAGTGAA |
| 2821 | GAATGAGAAC AGATTCTACC TCAAAGAGGT CAACATTTCA ATGTACCTGG TGAATGGGAG |
| 2881 | TGTGTTCTCC ATTGCTAACA ACAACCTGAG CTACTGGGAT GCCCCTCTGG GCTCCTCATA |
| 2941 | CATGTGCAAC AAGGAACAGA CTGTGAGTGT GTCAGGGGCC TTCCAGATCA ACACTTTTGA |
| 3001 | CCTGAGAGTG CAGCCCTTTA ATGTGACACA GGGAAAGTAC AGCACTGCTC AGGAGTGCAG |
| 3061 | CCTGGATGAT GACACTATCC TGATCCCTAT CATTGTGGGG GCAGGCCTGT CTGGACTCAT |
| 3121 | TATTGTGATT GTGATTGCCT ATGTGATAGG GAGAAGGAAG TCTTATGCTG GATACCAGAC |
| 3181 | CCTGTAAAAG GGCGAATTCC AGCACACGCG TCCTAGGAGC TCGAGTACTA CTGGCGGCCG |
| 3241 | TTACTAGTGG ATCCGCGGTA CAAGTAAGCA TGCAAGCTTC GAGGACGGGG TGAACTACGC |
| 3301 | CTGAATCAAG CTTATCGATA AATTCGAGCA TCTTACCGCC ATTTATTCCC ATATTTGTTC |
| 3361 | TGTTTTTCTT GATTTGGGTA TACATTTAAA TGTTAATAAA ACAAAATGGT GGGGCAATCA |
| 3421 | TTTACATTTT TAGGGATATG TAATTACTAG TTCAGGTGTA TTGCCACAAG ACAAACATGT |
| 3481 | TAAGAAACTT TCCCGTTATT TACGCTCTGT TCCTGTTAAT CAACCTCTGG ATTACAAAAT |
| 3541 | TTGTGAAAGA TTGACTGATA TTCTTAACTA TGTTGCTCCT TTTACGCTGT GTGGATATGC |
| 3601 | TGCTTTAATG CCTCTGTATC ATGCTATTGC TTCCCGTACG GCTTTCGTTT TCTCCTCCTT |
| 3661 | GTATAAATCC TGGTTGCTGT CTCTTTATGA GGAGTTGTGG CCCGTTGTCC GTCAACGTGG |
| 3721 | CGTGGTGTGC TCTGTGTTTG CTGACGCAAC CCCCACTGGC TGGGGCATTG CCACCACCTG |
| 3781 | TCAACTCCTT TCTGGGACTT TCGCTTTCCC CCTCCCGATC GCCACGGCAG AACTCATCGC |
| 3841 | CGCCTGCCTT GCCCGCTGCT GGACAGGGGC TAGGTTGCTG GGCACTGATA ATTCCGTGGT |
| 3901 | GTTGTCGGGG AAGGGCCTCG ATACCGTCGA TATCGATCCT GGCTAATAAA GGAAATTTAT |
| 3961 | TTTCATTGCA ATAGTGTGTT GGAATTTTTT GTGTCTCTCA CTCGGAAGGA CATATGGGAG |
| 4021 | GGCAAATCAT TTAAAACATC AGAATGAGTA TTTGGTTTAG AGTTTGGCAA CATATGCCCA |
| 4081 | TATGCTGGCT GCCATGAACA AAGGTTGGCT ATAAAGAGGT CATCAGTATA TGAAACAGCC |
| 4141 | CCCTGCTGTC CATTCCTTAT TCCATAGAAA AGCCTTGACT TGAGGTTAGA TTTTTTTTAT |
| 4201 | ATTTTGTTTT GTGTTATTTT TTTCTTTAAC ATCCCTAAAA TTTTCCTTAC ATGTTTTACT |
| 4261 | AGCCAGATTT TTCCTCCTCT CCTGACTACT CCCAGTCATA GCTGTCCCTC TTCTCTTATG |
| 4321 | GAGATCGAAG CAATTCGTTG ATCTGAATTT CGACCACCCA TAATAGATCT CCCATTACCC |
| 4381 | TGGTAGATAA GTAGCATGGC GGGTTAATCA TTAACTACAA GGAACCCCTA GTGATGGAGT |
| 4441 | TGGCCACTCC CTCTCTGCGC GCTCGCTCGC TCACTGAGGC CGGGCGACCA AAGGTCGCCC |
| 4501 | GACGCCCGGG CTTTGCCCGG GCGGCCTCAG TGAGCGAGCG AGCGCGCAG. |

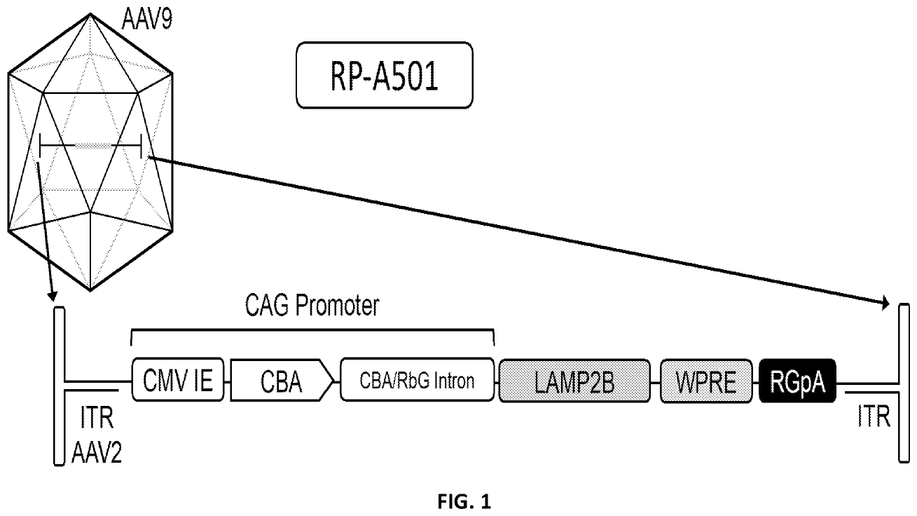

In certain embodiments, the expression cassette comprises one or more modifications as compared to a sequence selected from SEQ ID NOs: 8-10, including but not limited to any of the modifications disclosed herein. In particular embodiments, the one or more modifications comprise one or more of: removal of one or more (e.g., all) upstream ATG sequences, replacement of the Kozak sequence with an optimized consensus Kozak sequence or another Kozak sequence, including but not limited to any of those disclosed herein, and/or replacement of the polyadenylation sequence with a full-length polyadenylation sequence or another polyadenylation sequence, including but not limited to any of those disclosed herein. An illustrative configuration of genetic elements within these exemplary expression cassettes is depicted in FIG. 1.

In an embodiment, the vector is an adeno-associated virus (AAV) vector. In an embodiment, the expression cassette comprises inverted terminal repeat (ITR) sequences selected from SEQ ID NOs: 11 and 12:

| | (SEQ ID NO: 11) |
|---|---|
| 1 | CTGCGCGCTC GCTCGCTCAC TGAGGCCGCC CGGGCAAAGC CCGGGCGTCG GGCGACCTTT |
| 61 | GGTCGCCCGG CCTCAGTGAG CGAGCGAGCG CGCAGAGAGG GAGTGGCCAA CTCCATCACT |
| 121 | AGGGGTTCCT; |
| (SEQ ID NO: 12) | |
| 1 | AGGAACCCCT AGTGATGGAG TTGGCCACTC CCTCTCTGCG CGCTCGCTCG CTCACTGAGG |
| 61 | CCGGGCGACC AAAGGTCGCC CGACGCCCGG GCTTTGCCCG GGCGGCCTCA GTGAGCGAGC |
| 121 | GAGCGCGCAG. |

In related embodiments, the disclosure provides gene therapy vectors comprising an expression cassette disclosed herein. Generally, the gene therapy vectors described herein comprise an expression cassette comprising a polynucleotide encoding one or more isoforms of lysosome-associated membrane protein 2 (LAMP-2), and allows for the expression of LAMP-2 to partially or wholly rectify deficient LAMP-2 protein expression levels and/or autophagic flux in a subject in need thereof (e.g., a subject having Danon disease or another disorder characterized by deficient autophagic flux at least in part due to deficient LAMP-2 expression). In particular embodiments, the expression cassette comprises a polynucleotide sequence encoding LAMP-2 disclosed herein, e.g., SEQ ID NOs: 2-5, or a functional variant thereof. In some embodiments, the variant sequence has at least 90%, at least 95%, at least 98%, or at least 99% identity to any of SEQ ID NOs: 2-5. In some embodiments, the variant is a fragment of any of SEQ ID NOs: 2-5, e.g., a fragment having at least 50%, at least 60%, at least 70%, at least 80%, at least 90%, or at least 95% of the sequence of any of SEQ ID NOs: 2-5.

### Gene Therapy Vectors

Gene therapy vectors can be viral or non-viral vectors. Illustrative non-viral vectors include, e.g., naked DNA, cationic liposome complexes, cationic polymer complexes, cationic liposome-polymer complexes, and exosomes. Examples of viral vectors include, but are not limited to, adenoviral, retroviral, lentiviral, herpesvirus and adeno-associated virus (AAV) vectors.

In composition and methods according to the present disclosure, the viral vector generally is an AAV vector. AAV is a 4.7 kb, single stranded DNA virus. Recombinant vectors based on AAV are associated with excellent clinical safety, since wild-type AAV is nonpathogenic and has no etiologic association with any known diseases. In addition, AAV offers the capability for highly efficient gene delivery and sustained transgene expression in numerous tissues. By an “AAV vector” is meant a vector derived from an adeno-associated virus serotype, including without limitation, AAV1, AAV2, AAV3, AAV4, AAV5, AAV6, AAV7, AAV8, AAV9, AAV10, AAVrh.10, AAVrh.74, etc. AAV vectors can have one or more of the AAV wild-type genes deleted in whole or part, e.g., the rep and/or cap genes, but retain functional flanking inverted terminal repeat (ITR) sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g. by the insertion, deletion or substitution of nucleotides, as long as the sequences provide for functional rescue, replication and packaging. AAV vectors may comprise other modifications, including but not limited to one or more modified capsid protein (e.g., VP1, VP2 and/or VP3). For example, a capsid protein may be modified to alter tropism and/or reduce immunogenicity.

Immunogenicity against AAV is anticipated. Following administration of AAV therapy, the development of antibodies to the viral capsid and/or transgene have been reported in the literature (Mendell et al. New Engl. J. Med. 377:1713-1722 (2017); Rangaraj an et al. New Engl. J. Med. 377:2519-2530 (2017). Consistent with published literature, the studies conducted in the present disclosure similarly showed an increase in serum neutralizing antibodies to AAV9 at Days 30 and 91 post-dosing. The vector delivery resulted in characteristic binding antibody response to AAV9 capsid, but no significant anti-drug antibody (ADA) response was noted to LAMP2B, with no detectable consequences regarding transduction-mediated LAMP2B function in target tissues.

Recombinant vectors based on AAV are associated with excellent clinical safety, since wild-type AAV is nonpathogenic and has no etiologic association with any known diseases. In addition, AAV offers the capability for highly efficient gene delivery and sustained transgene expression in numerous tissues. Various serotypes of AAV are known, including, AAV1, AAV2, AAV3, AAV4, AAV5, AAV6, AAV7, AAV8, AAV9, AAV10, AAVrh.10, AAVrh.74, etc. AAV vectors can have one or more of the AAV wild-type genes deleted in whole or part, e.g., the rep and/or cap genes, but retain functional flanking inverted terminal repeat (ITR) sequences. The serotype of a recombinant AAV vector is determined by its capsid. International Patent Publication No. WO2003042397A2 discloses various capsid sequences including those of AAV1, AAV2, AAV3, AAV8, AAV9, and rh10. International Patent Publication No. WO2013078316A1 discloses the polypeptide sequence of the VP1 from AAVrh74. Numerous diverse naturally occurring or genetically modified AAV capsid sequences are known in the art.

An illustrative, non-limiting capsid is an AAV9 capsid, having the sequence of SEQ ID NO: 28 (or the VP1, VP2, or VP3 fragments thereof). In some embodiments, the AAV vectors of the disclosure comprise capsid proteins that share at least 93%, at least 94%, at least 95%, at least 96%, at least 97%, at least 98%, at least 99%, or at least 100% identity of the entire sequence of SEQ ID NO: 28, or over amino acids 138 to 736 of SEQ ID NO: 28, or over amino acids 203 to 736 of SEQ ID NO: 28.

| (SEQ ID NO: 28) | |
|---|---|
| 1 | MAADGYLPDW LEDNLSEGIR EWWALKPGAP QPKANQQHQD NARGLVLPGY KYLGPGNGLD |
| 61 | KGEPVNAADA AALEHDKAYD QQLKAGDNPY LKYNHADAEF QERLKEDTSF GGNLGRAVFQ |
| 121 | AKKRLLEPLG LVEEAAKTAP GKKRPVEQSP QEPDSSAGIG KSGAQPAKKR LNFGQTGDTE |
| 181 | SVPDPQPIGE PPAAPSGVGS LTMASGGGAP VADNNEGADG VGSSSGNWHC DSQWLGDRVI |
| 241 | TTSTRTWALP TYNNHLYKQI SNSTSGGSSN DNAYFGYSTP WGYFDFNRFH CHFSPRDWQR |
| 301 | LINNNWGFRP KRLNFKLFNI QVKEVTDNNG VKTIANNLTS TVQVFTDSDY QLPYVLGSAH |
| 361 | EGCLPPFPAD VFMIPQYGYL TLNDGSQAVG RSSFYCLEYF PSQMLRTGNN FQFSYEFENV |
| 421 | PFHSSYAHSQ SLDRLMNPLI DQYLYYLSKT INGSGQNQQT LKFSVAGPSN MAVQGRNYIP |
| 481 | GPSYRQQRVS TTVTQNNNSE FAWPGASSWA LNGRNSLMNP GPAMASHKEG EDRFFPLSGS |
| 541 | LIFGKQGTGR DNVDADKVMI TNEEEIKTTN PVATESYGQV ATNHQSAQAQ AQTGWVQNQG |
| 601 | ILPGMVWQDR DVYLQGPIWA KIPHTDGNFH PSPLMGGFGM KHPPPQILIK NTPVPADPPT |
| 661 | AFNKDKLNSF ITQYSTGQVS VEIEWELQKE NSKRWNPEIQ YTSNYYKSNN VEFAVNTEGV |
| 721 | YSEPRPIGTR YLTRNL. |

AAV expression vectors are constructed using known techniques to at least provide as operatively linked components in the direction of transcription, control elements including a transcriptional initiation region, the DNA of interest (i.e. the LAMP-2 gene) and a transcriptional termination region.

In some embodiments, the viral vector is an AAV9 vector. In some embodiments, the expression cassette of the viral vector is flanked by AAV2 inverted terminal repeats (ITRs). ITRs used in alternative embodiments of the disclosed vectors include, but are not limited to, AAV2, AAV3, AAV4, AAV5, AAV6, AAV7, AAV8, and AAV9. In some embodiments, the viral vector is an AAV2/9 vector. The notation AAV2/9 refers to an AAV vector have the ITRs of AAV2 and the capsid of AAV9. Other embodiments of the disclosure include without limitation AAV2/9, AAV5/9, AAVrh74, AAV2/rh74, AAV5/9, and AAV5/rh74 vectors. Other ITRs known in the art may be used. Exemplary ITRs (and other AAV components) useful in the vectors of the present disclosure include, without limitation, those described in U.S. Pat. Nos. 6,936,466B2, 9,169,494B2, US20050220766A1, US20190022249A1, and U.S. Pat. No. 7,282,199B2, which are each incorporated by reference herein in their entireties.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

The present disclosure also provides pharmaceutical compositions comprising an expression cassette or vector (e.g., gene therapy vector) disclosed herein and one or more pharmaceutically acceptable carriers, diluents or excipients. In particular embodiments, the pharmaceutical composition comprises an AAV vector comprising an expression cassette disclosed herein, e.g., wherein the expression cassette comprises a codon-optimized transgene encoding LAMP-2B, e.g., any of SEQ ID NOs: 3-5 and variants thereof. Provided are pharmaceutical compositions, e.g., for use in preventing or treating a disorder characterized by deficient autophagic flux (e.g., Danon disease) which comprises a therapeutically effective amount of an expression cassette or vector disclosed herein that comprises a nucleic acid sequence of a polynucleotide that encodes one or more isoforms of LAMP-2.

AAV vectors useful in the practice of the present invention can be packaged into AAV virions (viral particles) using various systems including adenovirus-based and helper-free systems. Standard methods in AAV biology include those described in Kwon and Schaffer. Pharm Res. (2008) 25(3):489-99; Wu et al. Mol. Ther. (2006) 14(3):316-27. Burger et al. Mol. Ther. (2004) 10(2):302-17; Grimm et al. Curr Gene Ther. (2003) 3(4):281-304; Deyle D R, Russell D W. Curr Opin Mol Ther. (2009) 11(4):442-447; McCarty et al. Gene Ther. (2001) 8(16):1248-54; and Duan et al. Mol Ther. (2001) 4(4):383-91. Helper-free systems included those described in U.S. Pat. Nos. 6,004,797; 7,588,772; and 7,094,604;

The pharmaceutical compositions that contain the expression cassette or vector may be in any form that is suitable for the selected mode of administration, for example, for intraventricular, intramyocardial, intracoronary, intravenous, intra-arterial, intra-renal, intraurethral, epidural or intramuscular administration. The gene therapy vector comprising a polynucleotide encoding one or more LAMP-2 isoforms can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the gene therapy vectors of the disclosure.

### Treatment of Danon Disease

Exemplary methods of treating lysosomal disorders and/or Danon disease are provided in WO 2018/170239 A1, which is incorporated herein in its entirety.

In an aspect, the disclosure provides methods of preventing, mitigating, ameliorating, reducing, inhibiting, eliminating and/or reversing one or more symptoms of Danon disease or another autophagy disorder in a subject in need thereof, wherein the method comprises administering to the subject a gene therapy vector of the disclosure. The term “Danon disease” refers to an X-linked dominant skeletal and cardiac muscle disorder with multisystem clinical manifestations. Danon disease mutations lead to an absence of lysosome-associated membrane protein 2 (LAMP-2) protein expression. Major clinical features include skeletal and cardiac myopathy, cardiac conduction abnormalities, cognitive difficulties, and retinal disease. Men are typically affected earlier and more severely than women.

Cardiac injection may be performed by central vein access, e.g., intrajugular vein. A Swan-Ganz pulmonary artery catheter (PAC) or other PAC may be used to deliver the AAV to the heart.

In an embodiment, the vector is administered via a route selected from the group consisting of parenteral, intravenous, intra-arterial, intracardiac, intracoronary, intramyocardial, intrarenal, intraurethral, epidural, and intramuscular. In an embodiment, the vector is administered multiple times. In an embodiment, the vector is administered by intramuscular injection of the vector. In an embodiment, the vector is administered by injection of the vector into skeletal muscle. In an embodiment, the expression cassette comprises a muscle-specific promoter, optionally a muscle creatine kinase (MCK) promoter or a MCK/SV40 hybrid promoter as described in Takeshita et al. Muscle creatine kinase/SV40 hybrid promoter for muscle-targeted long-term transgene expression. Int J Mol Med 2007 February; 19(2):309-15. In an embodiment, the vector is administered by intracardiac injection.

In an embodiment, the vector, e.g., AAV vector, is administered systemically, and more particularly, intravenously. Advantageously, the vector is administered at a dose (in vg per mL, vg/kg body mass, or vg/min/kg) less than the dose required to observe the same response when an original or wild-type LAMP-2B sequence is used. In particular embodiments, the vector is an AAV2/9 vector comprising an expression cassette comprising a polynucleotide encoding LAMP-2B disclosed herein.

In some embodiments, the disclosure provides a method of expressing LAMP-2B in a subject, comprising systemically administering an adeno-associated viral (AAV) vector to the subject, wherein the AAV vector comprises an expression cassette comprising a transgene sharing at least 95% identity with SEQ ID NO: 2 or is identical to SEQ ID NO: 2, the transgene operatively linked to an enhancer/promoter region, wherein systemic administration of the AAV vector to the subject results in increased expression of LAMP-2B compared to expression of LAMP-2B prior to administration of the AAV vector or expression of LAMP-2B in an untreated control subject. In some embodiments, the AAV virion is an AAV2/9 vector, which is a vector having an AAV9 capsid and AAV2 ITRs in the vector genome. In particular embodiments, the expression cassette comprises any of the elements disclosed herein. In some embodiment, systemic administration comprises intravenous administration. In some embodiments, the subject is exhibiting symptoms of Danon disease. In some embodiments, the subject suffers from, or is at risk for, Danon disease.

Systemic (or more particularly intravenous) administration in some embodiments results in expression of LAMP-2B polynucleotide as mRNA, in the form of an mRNA expressed from the transgene, in one or more tissues (e.g. heart, muscle, and/or liver) of the subject. In some embodiments, expression of the LAMP-2B polynucleotide as mRNA is increased at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.2-fold, at least about 2.3-fold, at least about 2.4-fold, at least about 2.5-fold, at least about 3-fold, or at least about 4-fold in the heart compared to expression in an untreated subject or a subject treated with a control vector. In some embodiments, expression of LAMP-2B polynucleotide as mRNA is increased at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.2-fold, at least 2.3-fold, at least 2.4-fold, at least 2.5-fold, at least 3-fold, or at least 4-fold in the heart compared to expression in an untreated subject or a subject treated with a control vector. In some embodiments, expression of LAMP-2B polynucleotide as mRNA is increased 1.2-fold, 1.3-fold, 1.4-fold, 1.5-fold, 1.6-fold, 1.7-fold, 1.8-fold, 1.9-fold, 2.0-fold, 2.2-fold, 2.3-fold, 2.4-fold, 2.5-fold, 3-fold, or 4-fold in the heart compared to expression in an untreated subject or a subject treated with a control vector.

In some embodiments, expression of LAMP-2B polynucleotide as mRNA is increased at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.2-fold, at least about 2.3-fold, at least about 2.4-fold, at least about 2.5-fold, at least about 3-fold, or at least about 4-fold in the muscle compared to expression in an untreated subject or a subject treated with a control vector. In some embodiments, expression of LAMP-2B polynucleotide as mRNA is increased at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.2-fold, at least 2.3-fold, at least 2.4-fold, at least 2.5-fold, at least 3-fold, or at least 4-fold in the muscle compared to expression in an untreated subject or a subject treated with a control vector. In some embodiments, expression of LAMP-2B polynucleotide as mRNA is increased 1.2-fold, 1.3-fold, 1.4-fold, 1.5-fold, 1.6-fold, 1.7-fold, 1.8-fold, 1.9-fold, 2.0-fold, 2.2-fold, 2.3-fold, 2.4-fold, 2.5-fold, 3-fold, or 4-fold in the muscle compared to expression in an untreated subject or a subject treated with a control vector.

In some embodiments, the LAMP-2B transgene is expressed in the heart and not expressed in the liver of the subject. In some embodiments, expression of LAMP-2B polynucleotide as mRNA is observed to be at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.2-fold, at least about 2.3-fold, at least about 2.4-fold, at least about 2.5-fold, at least about 3-fold, or at least about 4-fold in the heart compared to the liver. In some embodiments, expression of LAMP-2B polynucleotide as mRNA is observed to be at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.2-fold, at least 2.3-fold, at least 2.4-fold, at least 2.5-fold, at least 3-fold, or at least 4-fold in the heart compared to the liver. In some embodiments, expression of LAMP-2B polynucleotide as mRNA is observed to be 1.2-fold, 1.3-fold, 1.4-fold, 1.5-fold, 1.6-fold, 1.7-fold, 1.8-fold, 1.9-fold, 2.0-fold, 2.2-fold, 2.3-fold, 2.4-fold, 2.5-fold, 3-fold, or 4-fold in the heart compared to the liver.

In some embodiments, expression of wild-type or functional LAMP-2B protein is increased at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.2-fold, at least about 2.3-fold, at least about 2.4-fold, at least about 2.5-fold, at least about 3-fold, or at least about 4-fold in the heart compared to expression in an untreated subject or a subject treated with a control vector. In some embodiments, expression of wild-type or functional LAMP-2B protein is increased at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.2-fold, at least 2.3-fold, at least 2.4-fold, at least 2.5-fold, at least 3-fold, or at least 4-fold in the heart compared to expression in an untreated subject or a subject treated with a control vector. In some embodiments, expression of wild-type or functional LAMP-2B protein is increased 1.2-fold, 1.3-fold, 1.4-fold, 1.5-fold, 1.6-fold, 1.7-fold, 1.8-fold, 1.9-fold, 2.0-fold, 2.2-fold, 2.3-fold, 2.4-fold, 2.5-fold, 3-fold, or 4-fold in the heart compared to expression in an untreated subject or a subject treated with a control vector.

In some embodiments, expression of wild-type or functional LAMP-2B protein is observed to be at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.2-fold, at least about 2.3-fold, or at least 5-fold, in the heart compared to the liver. In some embodiments, expression of wild-type or functional LAMP-2B protein is observed to be at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.2-fold, at least 2.3-fold, at least 2.4-fold, at least 2.5-fold, at least 3-fold, or at least 4-fold in the heart compared to the liver. In some embodiments, expression of wild-type or functional LAMP-2B protein is observed to be 1.2-fold, 1.3-fold, 1.4-fold, 1.5-fold, 1.6-fold, 1.7-fold, 1.8-fold, 1.9-fold, 2.0-fold, 2.2-fold, 2.3-fold, 2.4-fold, 2.5-fold, 3-fold, or 4-fold in the heart compared to the liver.

In some embodiments, administration of the gene therapy vector results in expression of wild-type or functional LAMP-2B protein in the liver of at most about 1.1-fold, at most about 1.2-fold, at most about 1.3-fold, at most about 1.4-fold, at most about 1.5-fold, at most about 1.6-fold, at most about 1.7-fold, at most about 1.8-fold, at most about 1.9-fold, or at most about 2-fold increased compared to expression in the liver of an untreated subject. In some embodiments, administration of the gene therapy vector results in expression of wild-type or functional LAMP-2B protein in the liver of at most 1.1-fold, at most 1.2-fold, at most 1.3-fold, at most 1.4-fold, at most 1.5-fold, at most 1.6-fold, at most 1.7-fold, at most 1.8-fold, at most 1.9-fold, or at most 2-fold increased compared to expression in the liver of an untreated subject. In some embodiments, administration of the gene therapy vector results in expression of wild-type or functional LAMP-2B protein in the liver of 1.1-fold, 1.2-fold, 1.3-fold, 1.4-fold, 1.5-fold, 1.6-fold, 1.7-fold, 1.8-fold, 1.9-fold, or 2-fold increased compared to expression in the liver of an untreated subject.

In an embodiment, the disclosure provides a method of treating a disease or disorder, optionally Danon disease, in a subject in need thereof, comprising contacting cells with a gene therapy vector according to the present disclosure and administering the cells to the subject. In an embodiment, the cells are stem cells, optionally pluripotent stem cells. In an embodiment, the stem cells are capable of differentiation into cardiac tissue. In an embodiment, the stem cells are capable of differentiation into muscle tissue, e.g., cardiac muscle tissue and/or skeletal muscle tissue. In an embodiment, the stem cells are autologous. In an embodiment, the stem cells are induced pluripotent stem cells (iPSCs).

In an embodiment, the disease or disorder is an autophagy disorder. In some embodiments, the autophagy disorder is selected from the group consisting of, but not limited to, end-stage heart failure, myocardial infarction, drug toxicities, diabetes, end-stage renal failure, and aging. In an embodiment, the subject is a mammal, e.g., a human. In an embodiment, the subject is exhibiting symptoms of Danon disease or another autophagy disorder. In an embodiment, the subject has been identified as having reduced or non-detectable LAMP-2 expression. In an embodiment, the subject has been identified as having a mutated LAMP-2 gene.

Subjects/patients amenable to treatment using the methods described herein include, but are not limited to, individuals at risk of a disease or disorder characterized by insufficient autophagic flux (e.g., Danon disease as well as other known disorders of autophagy including, but not limited to, systolic and diastolic heart failure, myocardial infarction, drug toxicities (for example, anthracyclines chloroquine and its derivatives), diabetes, end-stage renal disease, and aging) but not showing symptoms, as well as subjects presently showing symptoms. Such subject may have been identified as having a mutated LAMP-2 gene or as having reduced or non-detectable levels of LAMP-2 expression.

In some embodiments, the patient is a human. In some embodiments, the patient is a pediatric, adolescent, or adult human. In some embodiments, the patient is 1, 2, 3, 4, 5, 6, 7, 8, 9, 10, 11, 12, 13, 14, 15, 16, 17, 18, 19, or 20 years old, or more than 20 years old. In some embodiments, the patient is 20 to 50 years old. In some embodiments, the patient is 50 to 65 years old. In some embodiments, the patient is 1 to 5, 2 to 6, 3 to 7, 4 to 8, 5 to 9, 6 to 10, 7 to 11, 8 to 12, 9 to 13, 10 to 14, 11 to 15, 12 to 16, 13 to 17, 14 to 18, 15 to 19, or 16 to 20 years old. In some embodiments, the patient is 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, or 20 to 21 years old. In a particular embodiment, the patient is 15 to 16 years old.

In some embodiments, the patient is a human male. In some embodiments, the patient is a pediatric, adolescent, or adult human male. In some embodiments, the patient is a 1, 2, 3, 4, 5, 6, 7, 8, 9, 10, 11, 12, 13, 14, 15, 16, 17, 18, 19, or 20 years old male, ora more than 20 years old male. In some embodiments, the patient is a 20 to 50 years old male. In some embodiments, the patient is a 50 to 65 years old male. In some embodiments, the patient is a 1 to 5, 2 to 6, 3 to 7, 4 to 8, 5 to 9, 6 to 10, 7 to 11, 8 to 12, 9 to 13, 10 to 14, 11 to 15, 12 to 16, 13 to 17, 14 to 18, 15 to 19, or 16 to 20 years old male. In some embodiments, the patient is a 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, or 20 to 21 year old male. In a particular embodiment, the patient is 15 to 16 years old.

In some embodiments, the patient is a human female. In some embodiments, the patient is a pediatric, adolescent, or adult human female. In some embodiments, the patient is a 1, 2, 3, 4, 5, 6, 7, 8, 9, 10, 11, 12, 13, 14, 15, 16, 17, 18, 19, or 20 years old female, or a more than 20 years old female. In some embodiments, the patient is a 20 to 50 years old female. In some embodiments, the patient is a 50 to 65 years old female.

In some embodiments, the subject is exhibiting symptoms of a disease or disorder characterized by insufficient autophagic flux (e.g., Danon disease as well as other known disorders of autophagy including, but not limited to, systolic and diastolic heart failure, myocardial infarction, drug toxicities, diabetes, end-stage renal disease, and aging). The symptoms may be actively manifesting, or may be suppressed or controlled (e.g., by medication) or in remission. The subject may or may not have been diagnosed with the disorder, e.g., by a qualified physician.

In some embodiments, the viral vector (e.g. AAV vector), or a pharmaceutical composition comprising that vector, is effective when administered systemically. For example, the viral vectors of the disclosure, in some cases, demonstrate efficacy when administered intravenously to subject (e.g., a primate, such as a non-human primate or a human). In some embodiments, the viral vectors of the disclosure are capable of inducing expression of LAMP-2B in various tissues when administered systemically (e.g., in heart, muscle, and/or lung). In particular embodiments, administration of an AAV9 vector comprising a transgene substantially identical to, or identical to, SEQ ID NO: 2 to a subject intravenously results in detectable expression of LAMP-2B in heart tissue. In some embodiments, expression of LAMP-2B is detectable in one or more, or all, of the left ventricle, the right ventricle, the left atrium, and the right atrium of the heart of the subject. In some embodiments, expression of LAMP-2B is detectable in sub-region 1 and/or sub-region 2 of the left ventricle of the heart of the subject.

“Detectable expression” typically refers to transgene expression at least 5%, 10%, 15%, 20% or more compared to a control subject or tissue not treated with the viral vector. In some embodiments, detectable expression means expression at least 1.5-fold, at least 2-fold, at least 2.5-fold, at least 3-fold, at least 5-fold, at least 10-fold, at least 20-fold, at least 50-fold, or at least 100-fold greater than a no-vector control. Transgene expression can be determined as the increase over expression of the wild-type or endogenous gene in the cell (accounting for the potential that expression of the transgene may influence expression of the endogenous gene). Transgene expression can also be determined by RT-PCR detection of sequences that are present on the transgene mRNA transcript but not on the mRNA transcript of the endogenous gene. For example, the 3′ UTR of the transcript may be used to determine the expression of the transgene independent of the expression of the endogenous gene (which may have a different 3′ UTR). Expression of the polypeptide encoded by the transgene can be assessed by western blot or enzyme-linked immunosorbent assay (ELISA), as described in the examples that follow, or other methods known in the art. Antibodies cross-reactive to the wild-type and exogenous copies of the protein may be used. In some cases, an antibody specific to the exogenous protein can be identified and used to determine transgene expression. Those of skill in the art can design appropriate detection methodologies taking into account the target cell or tissue. In some cases, expression is measured quantitatively using a standard curve. Standard curves can be generated using purified LAMP-2 protein, by methods described in the examples or known in the art. Alternatively, expression of the transgene can be assessed by quantification of the corresponding mRNA.

As used herein, the terms “vector genome” and “genome copies” refer, interchangeably, to the number of single-stranded AAV genome polynucleotides in a sample. Vector genome copies can be measured using quantitative polymerase chain reaction (qPCR) or digital droplet polymerase chain reaction (ddPCR) using primers specific to the recombinant AAV genome, such as primers flanking the WPRE sequence of the genome. Quantification may be made with respect to a standard curve generated with a reference sample, such as a sample containing plasmid DNA bearing the target amplicon for the primers used. Methods of ddPCR and qPCR are well known in the art. The dose units for preclinical studies are expressed in vector genomes (vg) per kg body weight. The clinical doses are expressed as vector genome copies (GC) per kg. Both of unit terminologies (GC/kg and vg/kg) are intended to describe the same entity and are used interchangeably in the present disclosure.

In some embodiments, detectable expression of LAMP-2B in heart tissue occurs at doses, in vector genomes (vg) per kilogram weight of subject (kg), of 5×10¹⁴ vg/kg or less, 3×10¹⁴ vg/kg or less, 2×10¹⁴ vg/kg or less, 1×10¹⁴ vg/kg or less, 9×10¹³ vg/kg or less, 8×10¹³ vg/kg or less, 7×10¹³ vg/kg or less, 6×10¹³ vg/kg or less, 5×10¹³ vg/kg or less, 4×10¹³ vg/kg or less, 3×10¹³ vg/kg or less, 2×10¹³ vg/kg or less, or 1×10¹³ vg/kg or less.

In some embodiments, detectable expression of LAMP-2B in heart tissue occurs at doses, in vector genomes (vg) per kilogram weight of subject (kg), of 1×10¹³ vg/kg to 2×10¹³ vg/kg, 2×10¹³ vg/kg to 3×10¹³ vg/kg, 3×10¹³ vg/kg to 4×10¹³ vg/kg, 4×10¹³ vg/kg to 5×10¹³ vg/kg, 5×10¹³ vg/kg to 6×10¹³ vg/kg, 6×10¹³ vg/kg to 7×10¹³ vg/kg, 7×10¹³ vg/kg to 8×10¹³ vg/kg, 8×10¹³ vg/kg to 9×10¹³ vg/kg, 9×10¹³ vg/kg to 1×10¹⁴ vg/kg, 1×10¹⁴ vg/kg to 2×10¹⁴ vg/kg, 2×10¹⁴ vg/kg to 3×10¹⁴ vg/kg, or 3×10¹⁴ vg/kg to 5×10¹⁴ vg/kg.

In some embodiments, detectable expression of LAMP-2B in heart tissue occurs at doses, in vector genomes (vg) per kilogram weight of subject (kg), of 1×10¹³ vg/kg to 3×10¹³ vg/kg, 3×10¹³ vg/kg to 5×10¹³ vg/kg, 5×10¹³ vg/kg to 7×10¹³ vg/kg, 7×10¹³ vg/kg to 9×10¹³ vg/kg, 9×10¹³ vg/kg to 2×10¹⁴ vg/kg, or 2×10¹⁴ vg/kg to 5×10¹⁴ vg/kg. In some embodiments, detectable expression of LAMP-2B in heart tissue occurs at doses, in vector genomes (vg) per kilogram weight of subject (kg), of 1×10¹³ vg/kg to 5×10¹³ vg/kg, 5×10¹³ vg/kg to 9×10¹³ vg/kg, 9×10¹³ vg/kg or to 5×10¹⁴ vg/kg. In some embodiments, detectable expression of LAMP-2B in heart tissue occurs at doses, in vector genomes (vg) per kilogram weight of subject (kg), of 1×10¹³ vg/kg to 9×10¹³ vg/kg, or 9×10¹³ vg/kg or to 5×10¹⁴ vg/kg.

In some embodiments, detectable expression of LAMP-2B in heart tissue occurs at doses, in vector genomes (vg) per kilogram weight of subject (kg), of 1×10¹³ vg/kg to 5×10¹³ vg/kg, 5×10¹³ vg/kg to 1×10¹⁴ vg/kg, or 1×10¹⁴ vg/kg to 5×10¹⁴ vg/kg.

In some embodiments, detectable expression of LAMP-2B in heart tissue occurs at doses, in vector genomes (vg) per kilogram weight of subject (kg), of 1×10¹³ vg/kg to 5×10¹⁴ vg/kg. In some embodiments, detectable expression of LAMP-2B in heart tissue occurs at doses, in vector genomes (vg) per kilogram weight of subject (kg), of 1×10¹³ vg/kg to 1×10¹⁴.

### Co-Administration

Safety and/or efficacy may be increase, in some cases, by co-administration of one or more secondary agents, including but not limited to immunomodulatory agents.

In some embodiments, the method comprises administering to the subject an effective amount of corticosteroid, including without limitation dexamethasone, methylprednisolone, or prednisone. Appropriate dosages and dose regimens of corticosteroid for administration of AAV therapy are known in the art. See Diehl et al. Cell. & Mol. Immunol. 14, 146-179 (2017).

In some embodiments, the method comprises administering to the subject an effective amount of tacrolimus, cyclosporine, rapamycin, sirolimus, or a derivative thereof. In some embodiments, the method further comprises administering to the subject an effective amount of corticosteroids prior to administering the effective amount of tacrolimus. As disclosed herein, tacrolimus may in some cases permit more rapid taper of corticosteroid levels after administrating to the subject of the AAV. Tacrolimus may be administered 7-21 days prior to the AAV, e.g. 21 days, 14 days, 10 days, 7 days, 5 days, 2 days, or 1 day before the AAV, preferably 1 day before. Tacrolimus administration may be continued after administration of the AAV. Tacrolimus may be administered for the duration of 120 days subsequent to the AAV, e.g. 1 day, 5 days, 10 days, 20 days, 30 days, 40 days, 50 days, 60 days, 70 days, 80 days, 90 days, 100 days, 110 days, or for 120 days after the AAV, preferably for 90 days after. See Tardieu et al. Hum. Gen. Ther. 25(6):506-516 (2014).

Tacrolimus may be administered at a dose of 0.01 mg/kg, 0.05 mg/kg, 0.1 mg·kg, 0.2 mg/kg, 0.3 mg/kg, 0.4 mg/kg, 0.5 mg/kg, or 0.6 mg/kg. Tacrolimus may be administered concurrently with 700 mg/m2, 800 mg/m2, 900 mg/m2, 1000 mg/m2, 1100 mg/m2, 1200 mg/m2, 1300 mg/m2, 1400 mg/m2, 1500 mg/m2, 1600 mg/m2, or 1700 mg/m2 mycophenolate mofetil.

In some embodiments, the method comprises administering to the subject 0.2 mg/kg tacrolimus concurrently with 1200 mg/m2 mycophenolate mofetil.

Tacrolimus may be administered per oral daily in 2 divided doses. Tacrolimus may be administered at an effective amount to maintain trough serum levels of 2 ng/mL, 2.5 ng/mL, 3 ng/mL, 3.5 ng/ml, 4 ng/mL, 4.5 ng/mL, or 5 ng/mL. Alternatives to tacrolimus include, without limitation mycophenolate, cyclosporine, cyclosporine modified, sirolimus, everolimus, or belatacept.

In some embodiments, the method comprises administering to the subject an effective amount of rituximab. As disclosed herein, rituximab may in some cases reduce and/or prevent an immune response to the AAV. Rituximab may be administered 7-21 days prior to the AAV, e.g. 21 days, 14 days, 10 days, 7 days, 5 days, 2 days, or 1 day before the AAV, preferably 1 day before. Rituximab may be administered 7-21 days after to the AAV, e.g. 21 days, 14 days, 10 days, 7 days, 5 days, 2 days, or 1 day after the AAV, preferably 1 day after. Rituximab may be administration 1, 2, 3, or more times to the subject. See Corti et al. Hum. Gene Ther. Clin. Dev. 28(4):208-218 (2017) and Corti et al. Mol. Ther. Meth. Clin. Dev. 1, 14033 (2014).

Rituximab may be administered at a dose of 300 mg/m2, 400 mg/m2, 500 mg/m2, 600 mg/m2, 700 mg/m2, 750 mg/m2, 800 mg/m2, 900 mg/m2, 1000 mg/m2, 1100 mg/m2, or 1200 mg/m2, preferably 750 mg/m2. Alternatives to ritixumab include, without limitation obinutuzumab, Tuxima (rituximab-abbs), or lenalidomide.

Tacrolimus may be administered before rituximab. Tacrolimus may be administered concurrently with rituximab. Tacrolimus may be administered after rituximab. Rituximab administration may be discontinued while tacrolimus administration is continued.

In some embodiments, rituximab is administered on Days −14 and −7 prior to administration of AAV and tacrolimus is administered beginning Day −7 prior to administration of AVV through 3 months following administration of AAV.

In some embodiments, the method comprises administering to the subject an effective amount of eculizumab, ravulizumab, or another complement inhibitor. Methods of treatment with complement inhibitors are known in the art. See Zipfel et al. Front. Immunol. (2019). Eculizumab is approved for treatment of atypical hemolytic-uremic syndrome (aHUS).

In some embodiments, the subject is at risk for sequelae of complement activation, such as atypical hemolytic-uremic syndrome (aHUS), optionally aHUS resulting in reversible thrombocytopenia and/or acute kidney injury (AKI).

In some embodiments, the method further comprises administering to the subject an effective amount of rituximab; administering to the subject an effective amount of tacrolimus; and/or administering to the subject an effective amount of eculizumab.

Various corticosteroids known in the art may be used. In some embodiments, the method comprises administering to the subject an effective amount of dexamethasone, methylprednisolone, bethamethasone, prednisone, prednisolone, triamcinolone, hydrocortisone, cortisone, fludrocortisone, or a combination thereof.

### Pharmaceutical Compositions and Dosages

In another aspect, the disclosure provides pharmaceutical compositions. In various embodiments, the pharmaceutical compositions contain vehicles (e.g., carriers, diluents and excipients) that are pharmaceutically acceptable for a formulation capable of being injected. Exemplary excipients include a poloxamer. Formulation buffers for viral vectors (including AAV) general contains salts to prevent aggregation and other excipients (e.g. poloxamer) to reduce stickiness of the vector. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. Advantageously, the formulation is stable for storage and use when frozen (e.g. at less than 0° C., about −60° C., or about −72° C.).

In some embodiments, the pharmaceutical compositions comprises a buffer (e.g., a phosphate buffer) at a suitable concentration (e.g., 200 mM) and pH (e.g., pH 7.2±0.1) for administration to a subject. The pharmaceutical composition may include Poloxamer at a suitable concentration (e.g., 0.01%.). The pharmaceutical composition may be provided at the site of treatment0 as a frozen product. The final volume of the unit dose of the AAV may be determined, in whole or in part, on the patient weight, e.g., in kilograms (kg), and the calculated or experimentally determined level of vector genome (vg) copies of the AAV per volume, e.g., milliliter (mL), or the pharmaceutical composition. The pharmaceutical composition may be diluted as necessary to obtain a desired concentration or volume for injection.

In some embodiments, the pharmaceutical composition comprises 200 mM NaCl, 10 mM NaH₂PO4, 1% (w/v) sucrose, 0.01% Poloxamer 188, pH 7.2±0.1.

In some embodiments, the AAV vector is administered at a dose of between about 1×10¹² and about 5×10¹⁴ vector genomes (vg) of the AAV vector per kilogram (vg) of total body mass of the subject (vg/kg). In some embodiments, the AAV vector is administered at a dose of between about 1×10¹³ and about 5×10¹⁴ vg/kg. In some embodiments, the AAV vector is administered at a dose of between about 5×10¹³ and about 3×10¹⁴ vg/kg. In some embodiments, the AAV vector is administered at a dose of between about 5×10¹³ and about 1×10¹⁴ vg/kg. In some embodiments, the AAV vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, or less than about 5×10¹⁴ vg/kg.

In some embodiments, the AAV vector is administered at a dose of between about 6.7×10¹³ and 2×10¹⁴ vg/kg. In some embodiments, the AAV vector is administered at a dose of between about 6.7×10¹³ and about 1.1×10¹⁴ vg/kg.

In some embodiments, the AAV vector is administered at a dose of about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, or about 5×10¹⁴ vg/kg. In some embodiments, the AAV vector is administered at a dose of about 6.7×10¹³ vg/kg, about 1.1×10¹³ vg/kg, or about 2.0×10¹³ vg/kg.

In some embodiments, the AAV vector is administered at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg. In some embodiments, the AAV vector is administered at a dose of 6.7×10¹³ vg/kg, 1.1×10¹³ vg/kg, or 2.0×10¹³ vg/kg.

In some embodiments, the AAV vector is administered systemically at a dose of between about 1×10¹³ and 5×10¹⁴ vector genomes (vg) of the AAV vector per kilogram (vg) of total body mass of the subject (vg/kg). In some embodiments, the AAV vector is administered systemically at a dose of between about 1×10¹³ and 5×10¹⁴ vg/kg. In some embodiments, the AAV vector is administered systemically at a dose of between about 5×10¹³ and 3×10¹⁴ vg/kg. In some embodiments, the AAV vector is administered systemically at a dose of between about 5×10¹³ and 1×10¹⁴ vg/kg. In some embodiments, the AAV vector is administered systemically at a dose of less than about less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, or less than about 5×10¹⁴ vg/kg.

In some embodiments, the AAV vector is administered systemically at a dose of between about 6.7×10¹³ and 2×10¹⁴ vg/kg. In some embodiments, the AAV vector is administered at a dose of between about 6.7×10¹³ and about 1.1×10¹⁴ vg/kg.

In some embodiments, the AAV vector is administered systemically at a dose of about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, or about 5×10¹⁴ vg/kg. In some embodiments, the AAV vector is systemically administered at a dose of about 6.7×10¹³ vg/kg, about 1.1×10¹³ vg/kg, or about 2.0×10¹³ vg/kg.

In some embodiments, the AAV vector is administered systemically at a dose of 1×10¹³ vg/kg, 3×10¹³ vg/kg, 5×10¹³ vg/kg, 7×10¹³ vg/kg, 1×10¹⁴ vg/kg, 3×10¹⁴ vg/kg, or 5×10¹⁴ vg/kg. In some embodiments, the AAV vector is systemically administered at a dose of 6.7×10¹³ vg/kg, 1.1×10¹³ vg/kg, or 2.0×10¹³ vg/kg.

In some embodiments, the AAV vector is administered intravenously at a dose of between about 1×10¹³ and 5×10¹⁴ vector genomes (vg) of the AAV vector per kilogram (vg) of total body mass of the subject (vg/kg). In some embodiments, the AAV vector is administered intravenously at a dose of between about 1×10¹³ and 5×10¹⁴ vg/kg. In some embodiments, the AAV vector is administered intravenously at a dose of between about 5×10¹³ and 3×10¹⁴ vg/kg. In some embodiments, the AAV vector is administered intravenously at a dose of between about 1×10¹³ and 1×10¹⁴ vg/kg. In some embodiments, the AAV vector is administered intravenously at a dose of less than about less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, or less than about 5×10¹⁴ vg/kg.

In some embodiments, the AAV vector is administered intravenously at a dose of between about 6.7×10¹³ and 2×10¹⁴ vg/kg. In some embodiments, the AAV vector is administered at a dose of between about 6.7×10¹³ and about 1.1×10¹⁴ vg/kg.

In some embodiments, the AAV vector is administered intravenously at a dose of about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, or about 5×10¹⁴ vg/kg. In some embodiments, the AAV vector is intravenously administered at a dose of about 6.7×10¹³ vg/kg, about 1.1×10¹³ vg/kg, or about 2.0×10¹³ vg/kg.

In some embodiments, the AAV vector is administered intravenously at a dose of 1×10¹³ vg/kg, 3×10¹³ vg/kg, 5×10¹³ vg/kg, 7×10¹³ vg/kg, 1×10¹⁴ vg/kg, 3×10¹⁴ vg/kg, or 5×10¹⁴ vg/kg. In some embodiments, the AAV vector is intravenously administered at a dose of 6.7×10¹³ vg/kg, 1.1×10¹³ vg/kg, or 2.0×10¹³ vg/kg.

In some embodiments, a therapeutically effective amount of the AAV virion is between about 1×10¹² and about 5×10¹⁴ vector genomes (vg) of the AAV virion per kilogram (vg) of total body mass of the subject (vg/kg). In some embodiments, the therapeutically effective amount is between about 1×10¹³ and about 5×10¹⁴ vg/kg. In some embodiments, the therapeutically effective amount is between about 5×10¹³ and about 3×10¹⁴ vg/kg. In some embodiments, the therapeutically effective amount is between about 5×10¹³ and about 1×10¹⁴ vg/kg. In some embodiments, the therapeutically effective amount is less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, or less than about 5×10¹⁴ vg/kg.

In some embodiments, the therapeutically effective amount of the AAV virion is between about 6.7×10¹³ and 2×10¹⁴ vg/kg. In some embodiments, the AAV virion is administered at a dose of between about 6.7×10¹³ and about 1.1×10¹⁴ vg/kg.

In some embodiments, the therapeutically effective amount of the AAV virion is about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, or about 5×10¹⁴ vg/kg. In some embodiments, the therapeutically effective amount is about 6.7×10¹³ vg/kg, about 1.1×10¹³ vg/kg, or about 2.0×10¹³ vg/kg.

In some embodiments, the therapeutically effective amount of the AAV virion is 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg. In some embodiments, the therapeutically effective amount is 6.7×10¹³ vg/kg, 1.1×10¹³ vg/kg, or 2.0×10¹³ vg/kg.

### Definitions

The terms “lysosome-associated membrane protein 2” and “LAMP-2” interchangeably refer to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 300, 400, or more amino acids, or over the full-length, to an amino acid sequence encoded by a LAMP-2 nucleic acid (see, e.g., GenBank Accession Nos. NM_002294.2 (isoform A). NM_013995.2 (isoform B), NM_001122606.1 (isoform C)) or to an amino acid sequence of a LAMP-2 polypeptide (see e.g., GenBank Accession Nos. NP_002285.1 (isoform A), NP_054701.1 (isoform B), NP_001116078.1 (isoform C)); (2) bind to antibodies, e.g., polyclonal antibodies, raised against an immunogen comprising an amino acid sequence of a LAMP-2 polypeptide (e.g., LAMP-2 polypeptides described herein); or an amino acid sequence encoded by a LAMP-2 nucleic acid (e.g., LAMP-2 polynucleotides described herein), and conservatively modified variants thereof; (3) specifically hybridize under stringent hybridization conditions to an anti-sense strand corresponding to a nucleic acid sequence encoding a LAMP-2 protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a LAMP-2 nucleic acid (e.g., LAMP-2 polynucleotides, as described herein, and LAMP-2 polynucleotides that encode LAMP-2 polypeptides, as described herein).

The terms “lysosome-associated membrane protein 2B” and “LAMP-2B” interchangeably refer to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 300, 400, or more amino acids, or over the full-length, to an amino acid sequence encoded by a LAMP-2B nucleic acid (see e.g., NM_013995.2) or to an amino acid sequence of a LAMP-2B polypeptide (see e.g., NP_054701.1); (2) bind to antibodies, e.g., polyclonal antibodies, raised against an immunogen comprising an amino acid sequence of a LAMP-2B polypeptide (e.g., LAMP-2B polypeptides described herein); or an amino acid sequence encoded by a LAMP-2B nucleic acid (e.g., LAMP-2B polynucleotides described herein), and conservatively modified variants thereof; (3) specifically hybridize under stringent hybridization conditions to an anti-sense strand corresponding to a nucleic acid sequence encoding a LAMP-2B protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a LAMP-2B nucleic acid (e.g., LAMP-2B polynucleotides, as described herein, and LAMP-2B polynucleotides that encode LAMP-2B polypeptides, as described herein).

The term “functional variant” in respect to a protein (e.g. a LAMP-2B) refers to a polypeptide sequence, or a fragment of a polypeptide sequence having at least about 30, at least about 40, at least about 50, at least about 60, at least about 70, or at least about 80 amino acid resides, that retains one or more functional attributes of the protein. For example, a functional variant of LAMP-2B is a LAMP-2B (as defined herein) that retains one or more functions such as: (1) regulating human cardiomyocyte function (Chi et al. (2019) PNAS USA 116 (2) 556-565); (2) improving metabolic and physiological function in Danon disease (Adler et al. (2019) J. Am. College Cardiology S0735-1097(19)31295-1); and/or (3) autophagy (Rowland et al. (2016) J. Cell Sci. (2016) 129, 2135-2143).

LAMP-2B has a lumenal domain (residues 29-375), a transmembrane domain (residues 376-399), and a cytoplasmic domain (residues 400-410), see UniProt Accession No. P13473. LAMP-2B functions in include chaperone-mediated autophagy, a process that mediates lysosomal degradation of proteins in response to various stresses and as part of the normal turnover of proteins with a long biological half-live (Cuervo et al. Science 273:501-503(1996), Cuervo et al. J. Cell Sci. 113:4441-4450(2000), Bandyopadhyay et al. Mol. Cell. Biol. 28:5747-5763(2008), Li et al. Exp. Cell Res. 327:48-56(2014), Hubert et al. Biol. Open 5:1516-1529(2016)). LAMP-2B may target GAPDH and MLLT11 for lysosomal degradation. LAMP-2B may be required for the fusion of autophagosomes with lysosomes during autophagy. It has been suggested that cells that lack LAMP2 express normal levels of VAMP8, but fail to accumulate STX17 on autophagosomes, which is the most likely explanation for the lack of fusion between autophagosomes and lysosomes. LAMP-2B may be required for normal degradation of the contents of autophagosomes. LAMP-2B may be required for efficient MHCII-mediated presentation of exogenous antigens via its function in lysosomal protein degradation; antigenic peptides generated by proteases in the endosomal/lysosomal compartment are captured by nascent MHCII subunits (Crotzer et al. Immunology 131:318-330(2010)).

Functional variants of LAMP-2B therefore include fragments of LAMP-2B that are capable of mediating any of the foregoing functions. In some embodiments, the function fragment of LAMP-2B includes one or more of the lumenal, transmembrane, and cytoplasmic domains. In some embodiments, the functional variant of LAMP-2B comprises one or more C-terminal or N-terminal deletions with respect to native LAMP-2B. In some embodiments, the functional variant of LAMP-2B comprises one or more internal deletions with respect to native LAMP-2B.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., LAMP-2 polynucleotide or polypeptide sequence as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to LAMP-2 nucleic acids and proteins, the BLAST and BLAST 2.0 algorithms and the default parameters are used.

A “comparison window,” as used herein, includes reference to a segment of any one of the number of contiguous positions selected from the group consisting of 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402(1977), respectively. Software for performing BLAST analyses is publicly available through the National Center for Biotechnology Information (on the worldwide web at ncbi.nlm.nih.gov/).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

As used herein, “administering” refers to local and systemic administration, e.g., including enteral, parenteral, pulmonary, and topical/transdermal administration. Routes of administration for compounds (e.g., polynucleotide encoding one or more LAMP-2 isoforms) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial. Other modes of delivery include, but are not limited to, the use of liposomal formulations, intravenous infusion, transdermal patches, etc.

The term “correction” refers to a change in a clinical parameter relative to a baseline level in the subject that causes the parameter normalize to a level equal to or approximately equal to the level of that parameter observed in a person that does not have Danon disease.

The term “improvement” refers to a change in a clinical parameter relative to a baseline level in the subject that causes the parameter increase (or decrease) to a level substantially greater than (or less than) the level of that parameter observed in the subject prior to administration of treatment. For example, an improvement may include reduction in size or number of autophagic vacuoles in the heart of the subject.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the compounds (e.g., LAMP-2 polynucleotides) and/or analogs thereof and another active agent, refers to administration of the compound and/or analogs and the active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, e.g., 30% or 40% or greater, e.g., 50% or 60% or greater, e.g., 70% or 80% or 90% or greater) of their maximum serum concentration for any given dose.

The term “therapeutically effective amount” refers to the amount and/or dosage, and/or dosage regime of a gene therapy vector necessary to bring about the desired result e.g., increased expression of one or more LAMP-2 isoforms in an amount sufficient to reduce the ultimate severity of a disease characterized by impaired or deficient autophagy (e.g., Danon disease).

The term “effective amount” refers to the amount and/or dosage, and/or dosage regime of a gene therapy vector necessary to bring about the desired result, e.g., the immunosuppressive effect of an immunosuppresive drug.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The terms “treating” and “treatment” also include preventing, mitigating, ameliorating, reducing, inhibiting, eliminating and/or reversing one or more symptoms of the disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In certain embodiments, the reduction or elimination of one or more symptoms of pathology or disease can include, e.g., measurable and sustained increase in the expression levels of one or more isoforms of LAMP-2.

As used herein, the phrase “consisting essentially of refers to the genera or species of active pharmaceutical agents recited in a method or composition, and further can include other agents that, on their own do not have substantial activity for the recited indication or purpose.

The terms “subject,” “individual,” and “patient” interchangeably refer to a human subject.

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells.

The term “vector” is used herein (when appearing alone) to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication or reverse transcription in a cell, or may include sequences sufficient to allow integration into host cell DNA. “Vectors” include gene therapy vectors. As used herein, the term “gene therapy vector” refers to a vector (such as an AAV virion) capable of use in performing gene therapy, e.g., delivering a polynucleotide sequence encoding a therapeutic polypeptide to a subject. Gene therapy vectors may comprise a nucleic acid molecule (“transgene”) encoding a therapeutically active polypeptide, e.g., a LAMP-2B or other gene useful for replacement gene therapy when introduced into a subject. Useful vectors include, but are not limited to, viral vectors. The terms “AAV vector” and “AAV virion” are used interchangeably herein to refer to a vector genome packaged into an AAV capsid.

As used herein, the term “expression cassette” refers to a DNA segment that is capable in an appropriate setting of driving the expression of a polynucleotide (a “transgene”) encoding a therapeutically active polypeptide (e.g., LAMP-2B) that is incorporated in said expression cassette. When introduced into a host cell, an expression cassette inter alia is capable of directing the cell's machinery to transcribe the transgene into RNA, which is then usually further processed and finally translated into the therapeutically active polypeptide. The expression cassette can be comprised in a gene therapy vector. Generally, the term expression cassette excludes polynucleotide sequences 5′ to the 5′ ITR and 3′ to the 3′ ITR.

All patents, patent publications, and other publications referenced and identified in the present specification are individually and expressly incorporated herein by reference in their entirety for all purposes.

### EXAMPLES

### Example 1: Recombinant AAV9 Vector Expressing LAMP2B

Recombinant AAV9 vector expressing LAMP2B was generated through a 3-plasmid, helper virus-free system. Transient transfection of pAAV-LAMP2B transfer plasmid, pAAV-2/9 packaging plasmid, and pAd-Helper adenovirus helper plasmid into HEK293T producer cells generated recombinant AAV particles containing serotype 9 capsid proteins and AAV2 ITRs flanking a human LAMP2B expression cassette (AAV9.LAMP2B).

The structure of the AAV cis transfer plasmid (pAAV-LAMP2B) contains the transgene expression cassette flanked by viral ITR regions derived from AAV2 as depicted in FIG. 1. The expression cassette contains the human LAMP2B coding sequence driven by a chimeric promoter containing the CMV IE enhancer (CMV IEE), chicken β-actin (CBA) promoter, chimeric chicken β-actin and rabbit globin intron. The expression cassette also includes a woodchuck hepatitis virus post-transcriptional regulatory element (WPRE) and is terminated by the rabbit globin polyA signal (RGpA).

### Example 2: Preclinical In Vitro and In Vivo Efficacy Studies

In vitro studies were conducted in iPSC-derived cardiomyocytes from a patient with Danon Disease (DD) indicated a dose-dependent increase in LAMP2B expression and a beneficial effect on mitochondrial membrane potential, a key cellular feature of cardiomyocytes in DD. After demonstration of in vitro phenotypic correction, in vivo studies of LAMP-2B gene therapy were performed in a clinically relevant calorically-restricted LAMP2 knockout (KO) mouse model.

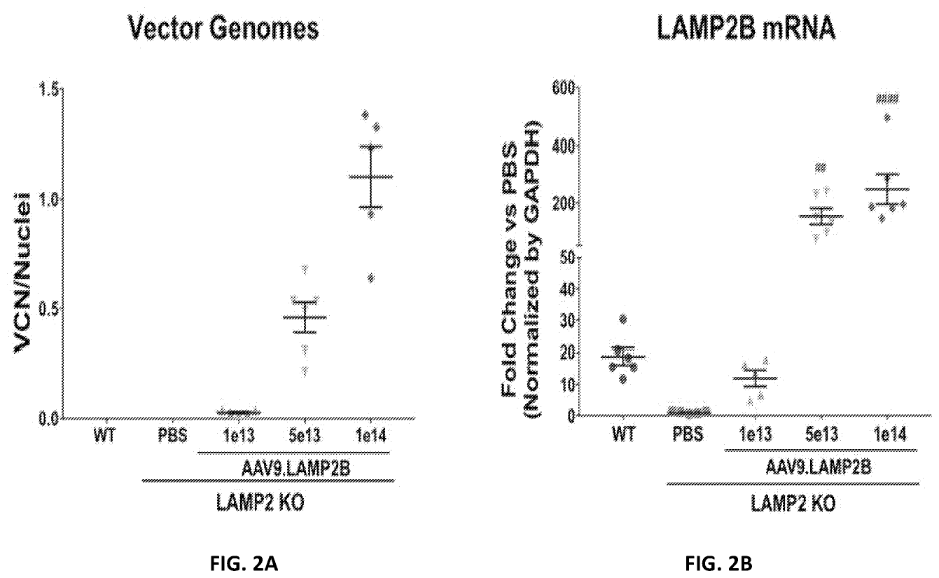

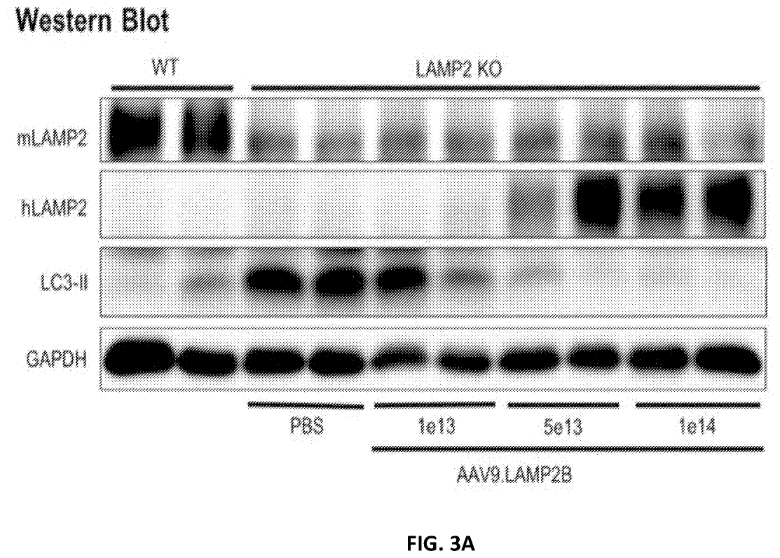

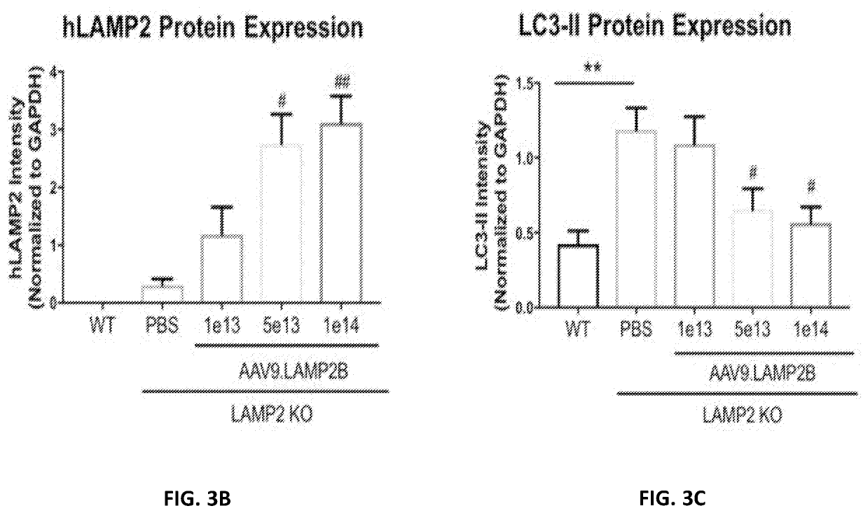

Lamp2 KO mice were intravenously injected with phosphate buffered saline (PBS) or AAV9.LAMP2B at doses of 1×10¹³, 5×10¹³, and 1×10¹⁴ vg/kg at 2 months of age and subjected to 6-weeks alternate fasting prior to their evaluation at 3 months post-treatment. As shown in FIGS. 2 and 3, dose-dependent increases in human LAMP2B and reductions in LC3-II (a marker of autophagic flux) were observed in organs particularly affected in DD including the heart, liver, and skeletal muscle of the AAV9-treated KO mice.

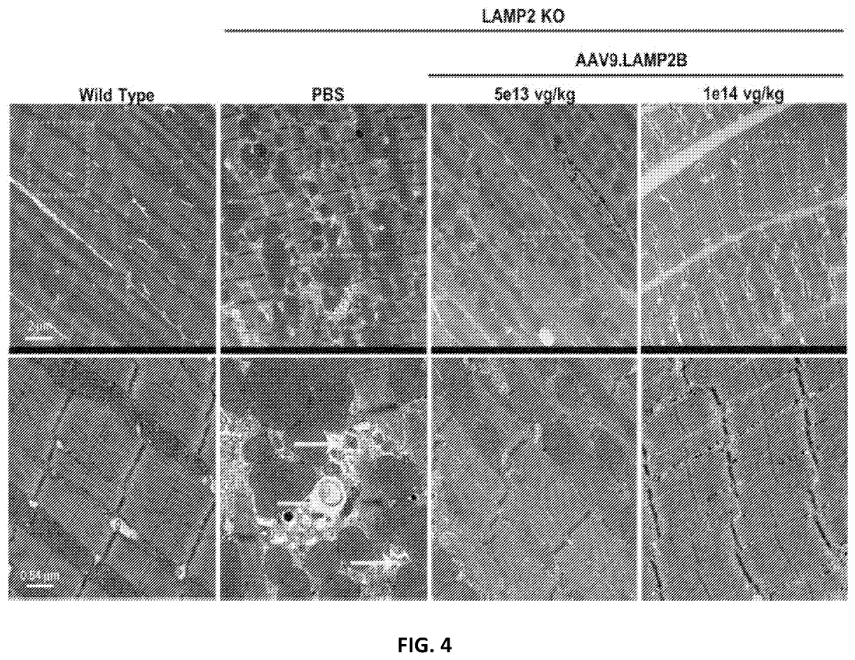

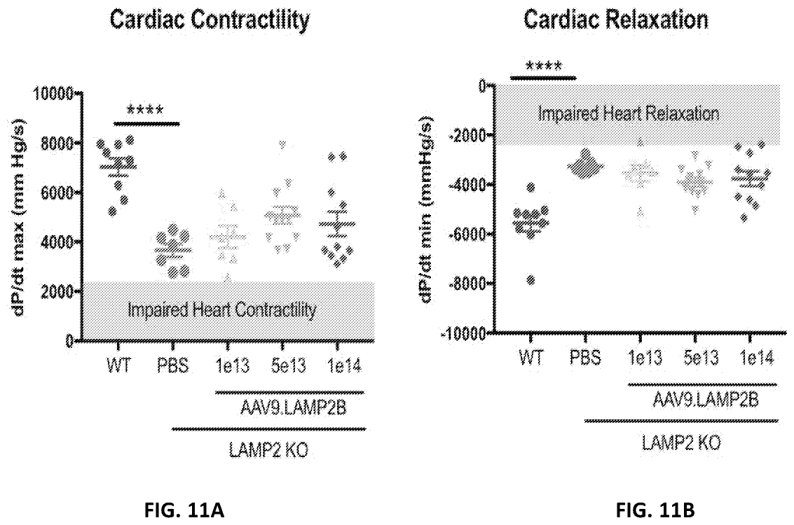

As shown in FIG. 4, AAV9.LAMP2B administration resulted in improved cardiac ultrastructure, including fewer visible autophagic vacuoles in Lamp2 KO mice treated at doses of 5×10¹³ and 1×10¹⁴ vg/kg 3 months after treatment. Cardiac ultrastructure from the Lamp2 KO mice cohort dosed at 1×10¹⁴ vg/kg was significantly improved and similar to the control WT animals. Conversely, KO mice that received only PBS showed an increased number of vacuoles in their cardiac tissue. To examine cardiac function, invasive hemodynamics were performed prior to study termination. As shown in FIG. 11, contractility was evaluated by invasive left intraventricular pressure (dP/dt max and dP/dt min) and found to be significantly decreased in untreated PBS-control Lamp2 KO mice compared to WT controls. Average dP/dt max and dP/dt min values in WT mice were 7050 and −5550 mmHg/s, respectively. In LAMP2 KO mice, average dP/dt max and dP/dt min values were: 3656 and −3265 mmHg/s for PBS (n=7), and 4197 and −3542 mmHg/s for 1×10¹³ vg/kg (n=7), 5073 and −3905 mmHg/s for 5×10¹³ vg/kg (n=12), and 4729 and −3765 mmHg/s for 1×10¹⁴ vg/kg (n=11) AAV9.LAMP2B treated groups.

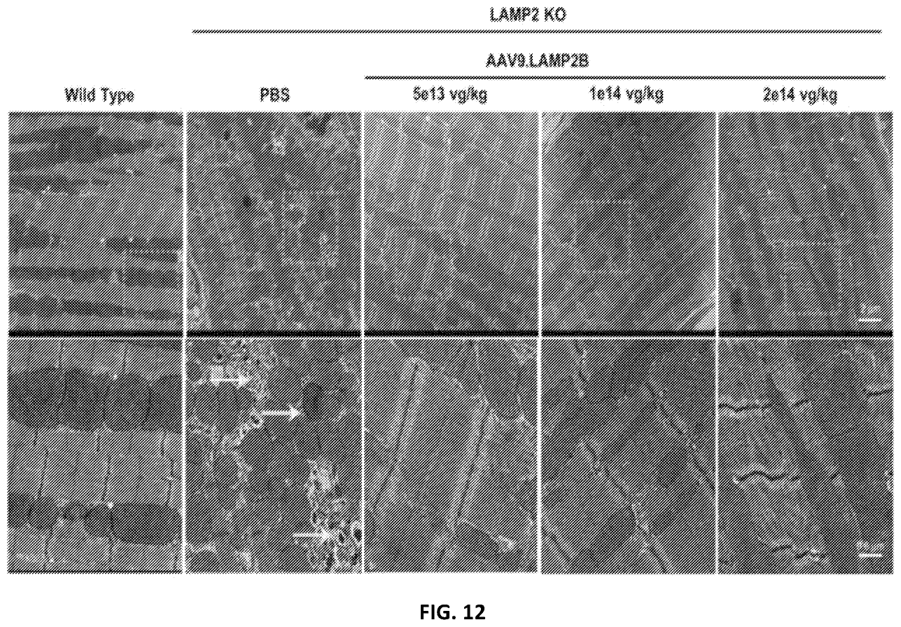

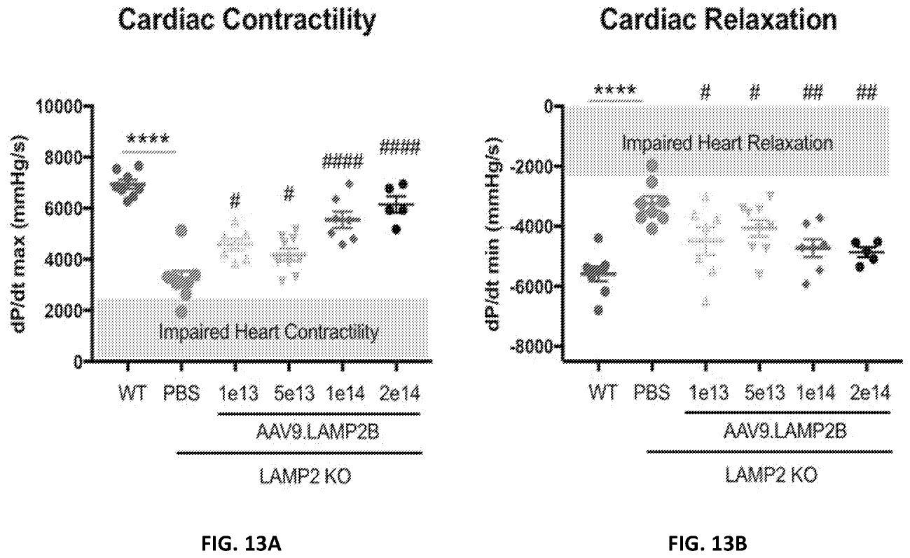

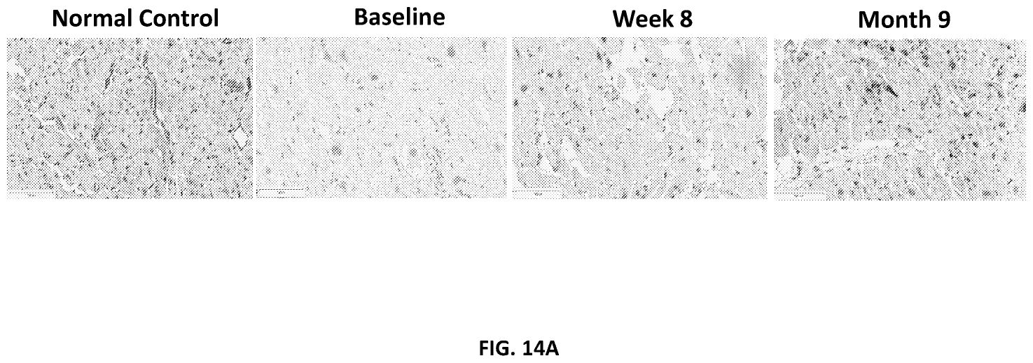

As shown in FIG. 12, AAV9.LAMP2B significantly improved the ultrastructure in hearts of adult Lamp2 KO mice. As shown in FIG. 13, a significant improvement in cardiac function was demonstrated by invasive hemodynamics at the 3-month time-point, and improvement was dose-dependent through the highest dose tested of 2×10¹⁴ vg/kg. At this highest dose, cardiac hemodynamics were comparable to the wild-type phenotype, thereby suggesting maximum possible efficacy and likely optimal efficacious dose. A significant reduction in hepatic enzymes suggested improved liver abnormality was also observed in the AAV9-dose animals. Results from this study indicated that AAV9.LAMP2B mediated gene therapy was safe and was successful at reversing the established disease phenotype by improving cardiac physiology, autophagic flux, and liver abnormality in older mice at an efficacious dose of 2×10¹⁴ vg/kg.

### Example 3: Preclinical Pharmacology

Safety measurements including clinical pathology, histopathology, and immune response to LAMP-2B gene therapy were performed in Lamp2 KO mice. A serum chemistry panel showed no adverse outcomes in mice that were either PBS controls or dosed with AAV9.LAMP2B vector. A significant increase in serum potassium (K+) levels was noted in the untreated PBS control KO mice relative to WT animals. These K+ levels were significantly reduced, returning to the expected range in mice treated with 5×10¹³ vg/kg, 1×10¹⁴ vg/kg and 2×10¹⁴ vg/kg of AAV9.LAMP2B compared to PBS-injected Lamp2 KO control mice. Elevated serum potassium in the setting of normal serum creatine and blood urea nitrogen levels may result from excessive muscle breakdown (Lehnhardt 2011). The observed reduction in serum potassium with AAV9.LAMP2B administration therefore shows a reduced myopathy in LAMP-2B gene therapy treated mice, an important feature of DD. Histopathology of the heart, liver, and skeletal muscle tissue showed no noteworthy changes to the AAV9.LAMP2B-treated samples. Biodistribution of vector was examined by qPCR, which revealed significant distribution among systemic organs with highest levels of vector genomes (vg) noted in liver with 10-fold less vg in heart and skeletal muscle and 100-fold less in brain compared to liver. The spleen and gonads exhibited the lowest amount of vector genome distribution. Quantitation of transgene expression by mRNA estimation was examined in a subset of tissues using RT-qPCR in mice dosed with 1×10¹⁴ vg/kg and 2×10¹⁴ vg/kg of AAV9.LAMP2B. On average, heart had the highest level of human LAMP2B mRNA followed closely by levels in liver and skeletal muscle. Brain, lungs, kidneys, and gonads also showed moderate levels of human LAMP2B mRNA, and very low levels were detected in the spleen at the 6-month timepoint post dosing.

The preclinical studies described herein were performed according to good laboratory practice (GLP) showed no unexpected mortality, physical, behavioral, morphologic, hematologic or biochemical abnormalities associated with doses up to 3×10¹⁴ vg/kg. Results from this study showed that AAV9.LAMP2B-mediated gene therapy demonstrated safety and durable benefit in the DD phenotype in Lamp2 KO mice. This study demonstrates a safe and effective profile for IV (intravenous) administration of LAMP-2B gene therapy and indicated minimal efficacious dose of AAV9.LAMP2B to be in the range of 5×10¹³ to 1×10¹⁴ vg/kg, demonstrating LAMP-2B gene therapy's clinical potential in humans for the treatment of Danon disease.

### Example 4: Preclinical Toxicology

Additionally, a 102-day non-GLP study was performed in 2-year-old non-human primates (NHPs) (cynomolgus monkeys) treated with the therapeutic vector at the highest dose level tested in the GLP murine toxicology study (3×10¹⁴ vg/kg). Animals were pre-screened for AAV9 neutralizing antibodies prior to shipment. All animals assigned to treatment and vehicle control groups demonstrated complete seronegativity (no virus neutralization at 1:5, 1:20, 1:80 dilutions). Two monkeys were assigned to the therapeutic vector group (3×10¹⁴ vg/kg) and 2 monkeys were assigned to the vehicle control group. Following dosing (intravenous injection into the saphenous vein), animals were evaluated at baseline and Days 3, 7, 15, 21, 30 42, 50, 60, 91, and 102. There was no unexpected mortality or significant changes in body weight. Blood samples were obtained from both cohorts at multiple timepoints for clinical pathology. A mild, transient transaminase increase at Day 7 and a concomitant transient decrease in platelets at Day 7 (within normal range) were observed in the AAV9.LAMP2B-injected group without any pathologic sequelae. Mild elevations (but within normal range) in creatinine kinase and lactate dehydrogenase at study Day 50 were also noted, with no accompanying clinical signs of toxicity. No other significant treatment-related effects were noted for any of the other hematologic or biochemical assessments performed.

### Example 5: LAMP-2B Gene Therapy Clinical Studies

An initial dose of 6.7×10¹³ GC/kg was selected for clinical study. The approach for dose escalation involves evaluation of intermediate doses between 6.7×10¹³ GC/kg and 2.0×10¹⁴ GC/kg.

The dose units for preclinical studies evaluating AAV9.LAMP2B are expressed in vector genomes (vg) per kg body weight. The clinical doses of AAV9.LAMP2B administered in this study are expressed as vector genome copies (GC) per kg, as this nomenclature is considered an optimal description of the investigational material as quantified in the manufacturing process. Both of these unit terminologies (GC/kg and vg/kg) are intended to describe the same entity with respect to transgene quantity.

The study will exclude subjects who have high pre-existing anti-AAV9 serum neutralizing antibody titers (Anti-AAV9 neutralizing antibody titer >1:40). Patients with evidence of synthetic or cholestatic hepatic dysfunction (PT/INR>1.5×ULN; bilirubin >1.5×ULN) will also be excluded (transaminase elevations up to 10×ULN or GGT up to 2×ULN are permitted because this is a prominent component of DD and are believed to predominantly reflect muscle aberrancies). Systemic corticosteroid therapy will be administered one day prior to AAV infusion, continued during the weeks following administration, and tapered to discontinuation between 8 and 12 weeks subsequent to infusion.

Safety monitoring included frequent testing of liver enzymes (including transaminases, bilirubin, ALP and coagulation parameters). Platelets and comprehensive coagulation profile (including PT/aPTT/fibrinogen/D-Dimer) will also be evaluated, as will complement pathway components. Serum and whole blood will also be obtained for evaluation of potential humoral and cell-mediated immune response against both viral capsid components and LAMP-2B.

### Objectives and Endpoints

### Primary Objectives

To characterize the safety and toxicity associated with infusion of a rAAV9 capsid containing the human LAMP2B transgene (investigational product).
To evaluate a range of single IV doses of investigational product with respect to safety, toxicity, and preliminary efficacy.
To determine if infusion of investigational product results in cardiomyocyte (and skeletal muscle) transduction and gene expression (as determined by assessment of myocardial LAMP2B DNA, RNA, and protein) via endomyocardial biopsy, to determine if there is correction of disease-associated histologic abnormalities (autophagic vacuoles, myofibrillar disarray), and to enable preliminary characterization of the extent of cardiomyocyte molecular and histologic correction.
To enable preliminary assessment of clinical stabilization, as determined by medical, radiographic, and cardiopulmonary exercise/physiologic parameters at approximately 8-12 weeks following investigational product infusion (similar evaluations at subsequent timepoints will be conducted to evaluate secondary objectives, as described subsequently).

### Secondary Objectives

To determine the percentage of patients for whom infusion of investigational product results in a sustained (6-month through 3-year following investigational product) improvement, stabilization (or reduced rate of deterioration versus historical controls) in cardiovascular pathophysiology, as determined by medical evaluation, radiographic evaluation of cardiac structure and function, and cardiopulmonary exercise/physiologic parameters.
To determine the percentage of patients in whom cardiomyocytes contain the corrected LAMP2B gene and/or protein and improvement in DD-associated histologic abnormalities, and when feasible to quantify the extent of genetic and histologic correction in the myocardium.
To determine and characterize immunologic response to investigational product (immunogenicity), including evaluation of humoral (antibody) and cellular (T-lymphocyte) anti-AAV9 and anti-LAMP-2B protein activity.
To evaluate the percentage of patients receiving investigational product who require and/or receive subsequent cardiac transplantation, LVAD, implantable cardioverter-defibrillator or pacemaker placement, electrophysiologic ablative procedure for cardiac conduction aberrancy, or subsequent hospitalizations for heart failure.
To evaluate overall survival in patients receiving investigational product, including 1- and 3-year overall survival assessment of all patients and specifically patients receiving investigational product at the dose selected for subsequent evaluation.

### Exploratory Objectives

To evaluate potential correlations between evidence of molecular and histologic correction in cardiomyocytes, with parameters of clinical stabilization or improvement, and to evaluate potential correlations between cardiomyocyte molecular/histologic correction and evidence of LAMP2B gene/protein in skeletal muscle and blood.
To evaluate serologic markers of muscle injury (including CPK and transaminases) and of congestive heart failure (including BNP, high-sensitivity troponin) and ascertain if earlier (i.e., 8-12 week) improvement in these blood markers may be potential surrogates of clinical, structural, and histologic modification of DD.
To evaluate patient-reported outcomes/quality-of-life (PRO/QOL) in patients receiving investigational product, as evaluated by means of the Kansas City Cardiomyopathy Questionnaire (KCCQ-12) and PedsQL.
To evaluate the presence and extent of improvement, stabilization (or reduced rate of deterioration versus historical controls) in non-cardiovascular aspects of DD, including assessment of neuromuscular and ophthalmologic function.

### Endpoints

### Safety Endpoints

The safety and tolerability endpoints are:
Incidence of treatment-emergent adverse events (TEAEs) and SAEs, overall and by intensity.
Incidence of TEAEs and SAEs, overall and by intensity, considered by the Investigator to be at least possibly related to AAV9.LAMP2B.
Proportion of patients requiring cardiac interventions, including cardiac transplant, implantable cardioverter-defibrillator, or pacemaker placement, electrophysiologic ablative procedure for cardiac conduction aberrancy, or subsequent hospitalizations for heart failure.
Characterization of immune response to AAV9.LAMP2B, as evidenced by antibodies or T-lymphocytes reactive to AAV-9 or to LAMP-2B protein.
Evidence of hepatotoxicity, based on changes from baseline in liver transaminases (AST and ALT), GGT, bilirubin, and ALP.
Evidence of coagulopathy, based on changes from baseline in platelet count, prothrombin time (PT, or International Normalized Ratio (INR)), activated partial thromboplastin time (aPTT), fibrinogen, D-dimer, thrombin-antithrombin complex (TAT), and complement components (complement 3 (C3), complement 4 (C4), and serum membrane attack complex (sC5b-9).
Changes from baseline in:
   Vital signs measurements.
   Safety laboratory test results.
   Physical examination findings.

### Efficacy Endpoints

The efficacy endpoints include assessments of clinical improvement, stabilization (or reduced rate of deterioration versus historical controls) in cardiovascular pathophysiology, as determined by medical evaluation, radiographic evaluation of cardiac structure and function, and cardiopulmonary exercise/physiologic parameters. Preliminary assessments of efficacy endpoints will occur during the initial safety-focused follow-up (initial 8-12 weeks following investigational product infusion) and during a more sustained (6-month through 3-year) follow-up period.

### Study Design

This is a non-randomized, open-label, Phase 1 study in patients with DD.

During this Phase 1 study, approximately 11-23 patients will receive a single IV infusion of investigational product (IP), with cohorts of patients receiving AAV9.LAMP2B at sequentially higher dose-levels according to the guidelines detailed below. Study sites that are not administering IP may participate in the trial. These sites would perform the initial screening and visits subsequent to IP dosing. The IP administration and immediate subsequent study visits are to be performed at the study site performing the IP administration. This is intended to reduce the burden of extensive travel for patients and their families.

Three dose levels are planned to be investigated in 6 distinct cohorts. Dosing of a cohort evaluating a given dose in a pediatric population (ages 8-14) will be feasible only when it is determined that fewer than 33% of patients within the comparable dose adult cohort have experienced dose-limiting toxicity (DLT). Dosing of cohorts in which adult or pediatric patients receive higher doses will be feasible only when it is determined that fewer than 33% of patients within a prior lower-dose cohort have experienced DLT. To be evaluable for DLT assessment, a patient must have received the intended dose of IP and remained available for follow-up during the 8 weeks subsequent to infusion of IP (with the exception of patients with fatal AEs considered related to investigational product during the initial 8 weeks subsequent to infusion).

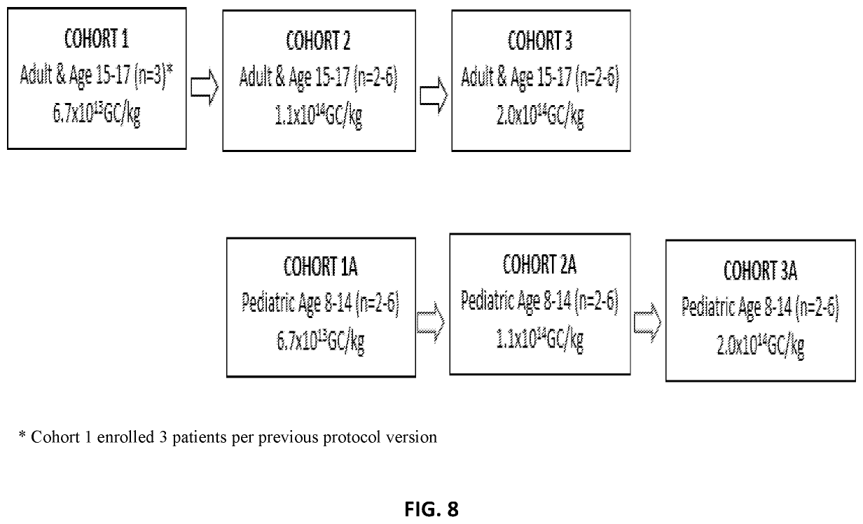

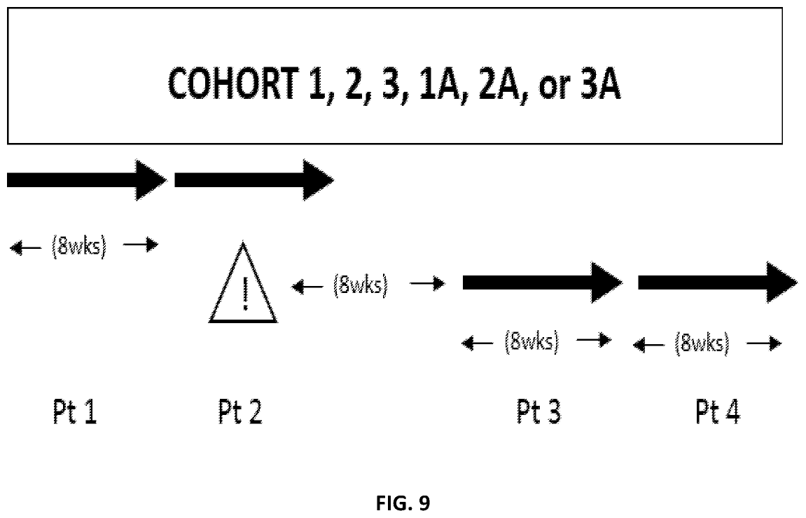

Pediatric patients (ages 8-14) at a given dose level will commence only pending determination of safety of the dose level in the older (adults and ages 15-17) cohort. FIG. 8 depicts the overall sequence of planned enrollment in the cohorts, according to a scenario in which DLT is not identified. FIG. 9 depicts the sequence of enrollment within any given cohort. Decisions regarding expansion of cohorts, opening of a subsequent cohort (involving an increased or otherwise modified investigational product dose), and recommendation of the dose(s) to be evaluated in subsequent clinical development will be made by the IDSMC based on the safety profile identified in prior cohorts and the prospect of direct benefit. Additional regularly scheduled reviews of investigational product safety by the IDSMC will occur subsequent to the initial 8-week DLT-evaluation periods for patients in each study cohort.

### Investigational Product

The Investigational Product (AAV9.LAMP2B) is gene therapy product consisting of an AAV9 capsid containing the human LAMP2B transgene with ITR elements, CAG promoter comprising CMV IEE, CBA promoter, CBA and rabbit globin introns, WPRE, and RGpA as shown in FIG. 1 and described in Example 1.

### Dosage Form

The composition of AAV9.LAMP2B consists of the active ingredient (recombinant AAV9.LAMP2B viral particles at a concentration of [3.0-6.0×10¹³ vg/mL] and capable of transducing target cells to express the therapeutic protein LAMP2B) formulated in buffer (200 mM NaCl, 10 mM NaH₂PO4, 1% (w/v) sucrose, 0.01% Poloxamer 188, pH 7.2±0.1) suitable for infusion. AAV9.LAMP2B is provided to the clinical site as a frozen product and the final volume of the dose of AAV9.LAMP2B is predicated on the patient weight in kilograms (kg) and the calculated vector genome (vg) copies of AAV9 per milliliter (mL).

### Doses to be Investigated

During this Phase 1 study, 11-23 patients will receive a single IV infusion of investigational product, with up to 3 specific cohorts of adult and pediatric patients receiving AAV9.LAMP2B at dose-levels according to the guidelines below. Activation of a cohort evaluating a given dose in a pediatric population (8-14) is only feasible when it is determined that fewer than 33% of patients within the comparable dose adult cohort have experienced a dose-limiting toxicity (DLT). Three AAV9.LAMP2B dose levels will be investigated according to a dose escalation design, as follows:
Cohort 1: Adult and age 15-17: 6.7×10¹³ GC/kg (n=3)*
Cohort 2: Adult and age 15-17: 1.1×10¹⁴ GC/kg (n=2-4)
Cohort 3: Adult and age 15-17: 2.0×10¹⁴ GC/kg (n=2-4)
Cohort 1A: Pediatric age 8-14: 6.7×10¹³ GC/kg (n=2-4)
Cohort 2A: Pediatric age 8-14: 1.1×10¹⁴ GC/kg (n=2-4)
Cohort 3A: Pediatric age 8-14: 2.0×10¹⁴ GC/kg (n=2-4)
Cohort 1 enrolled 3 patients per previous protocol version

Evaluation of AAV9.LAMP2B in pediatric patients (age 8-14) at a given dose level will commence only pending determination of safety of the dose level in the older (adults and those aged 15-17 and generally capable of providing assent) population. AAV9.LAMP2B will be administered at doses based on total body weight. If the patient is obese (body mass index (BMI)>85%, per the Centers for Disease Control and Prevention (CDC) growth chart, the IP may be administered at doses based on lean body mass (LBM) using the Hume formula below. LBM=(0.32810×W)+(0.33929×H)−29.5336

The study excluded patients with pre-existing anti-AAV9 serum neutralizing antibody titers (>1:40). Patients received prophylaxis for anti-AAV immunogenic response with rituximab, tacrolimus, and corticosteroids. Rituxumab was administered prior to IP infusion. Tacrolimus was administered for 3 months and started prior to IP administration. Corticosteroids started the day prior to administration of the therapeutic vector and then daily through Week 8 post-treatment, followed by a 4-week taper prior to discontinuation. The incorporation of tacrolimus and rituximab as part of the immunosuppressive regimen enables a reduced overall corticosteroid administration.

### Clinical Safety Profile for Cohort 1

A cohort of three patients 15 years and older (Cohort 1) received LAMP-2B gene therapy at a dose of 6.7×10¹³ GC/kg with concomitant corticosteroids as per protocol. Subject characteristics are shown in Table 1. The second and third patients also received tacrolimus. Treatment regimen and LAMP2B relative expression are shown in Table 2. No significant anti-drug antibody (ADA) response was noted to LAMP2B.

The patients showed some constitutional symptoms (such as nausea, vomiting, abdominal pain, and low grade fever) in the days after receiving IP. As expected, the patients developed an immune response subsequent to IP administration. This immune response was associated with decreased blood cell counts (platelets, white blood cells), elevated transaminases, elevated skeletal muscle- and heart-related enzymes and peptide levels. The decrease in platelet count that occurred approximately 1 to 2 weeks after treatment was associated with a corresponding increase in D-dimer and decreases in C3 and C4 (complement moieties). Decreases in platelet count have been observed in other AAV gene therapy programs and are consistent with an acute complement mediated immune reaction against the AAV9 capsid. The patients treated to date with LAMP-2B gene therapy were maintained on corticosteroids and were not treated with eculizumab. The observed changes in platelets, D-dimer and C3/C4 levels improved after several days.

Increases in aspartate transaminase (AST) and alanine aminotransferase (ALT) have been demonstrated in other AAV gene therapy programs. The AST/ALT increases observed were not associated with increases in bilirubin above normal range and with clinical signs/symptoms of hepatobiliary disease. Elevations in GGT (a liver enzyme that is not affected by injury to skeletal muscle, unlike AST/ALT) were more modest, and did not exceed levels greater than 5× baseline. The EliSpot assay has been negative for both the AAV9 capsid and the LAMP2B transgene for the first three patients in Cohort 1. Because the GGT increases from baseline have been proportionally lower than the AST and ALT increases from baseline, these increases in AST and ALT are predominantly skeletal muscle in origin. This is also seen with the associated elevations of CPK. Two of the three patients in Cohort 1 developed a skeletal muscle weakness consistent with a steroid induced myopathy. The skeletal muscle weakness recovered as the corticosteroids were tapered to discontinuation. At 6 months, 9 months, and 12 months post-treatment, the patients are stable at home.

TABLE 1

| | Age at | Dosing | | |
|---|---|---|---|---|
| Patient ID | Treatment | Weight | Cohort Dose | Total Dose |
| A501-005-1001 | 17-years | 52.2 kg | 6.7 × 1013 GC/kg | 3.25 × 1015 GC |
| A501-005-1002 | 20-years | 89.1 kg | 6.7 × 1013 GC/kg | 5.97 × 1015 GC |
| A501-005-1005 | 18-years | 97.8 kg | 6.7 × 1013 GC/kg | 6.08 × 1015 GC |

TABLE 2

| | | LAMP2B Relative Expression vs. Normal* | | | |
|---|---|---|---|---|---|
| Subject | Regimen | Baseline | Week 8 | Month 6 | Month 12 |
| Subject 1001 | Steroids | 1.0% | 7.8% | 9.4% | 2.6% |
| | only | | | | |
| | (limited | | | | |
| | compliance) | | | | |
| Subject 1002 | Steroids | 8.9% | 36.9% | ND | 67.8% |
| | only (local | | | | |
| | monitoring) | | | | |
| Subject 1005 | Steroids → | 3.3% | 17.6% | 92.4%** | January 2021 |
| | Tacrolimus | | | | |

*Percent area of cell staining was quantitated using software in a blinded fashion and expression compared to normal heart tissue. Values represent average of 3-14 sections.
**Sample taken at Month 9

### Gene Expression and Efficacy Endpoints of Cohort 1

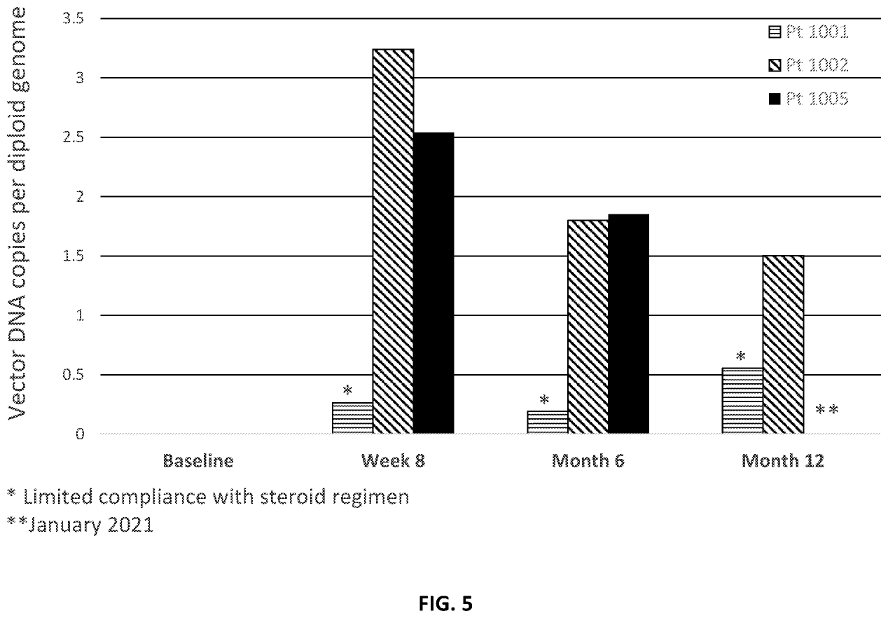

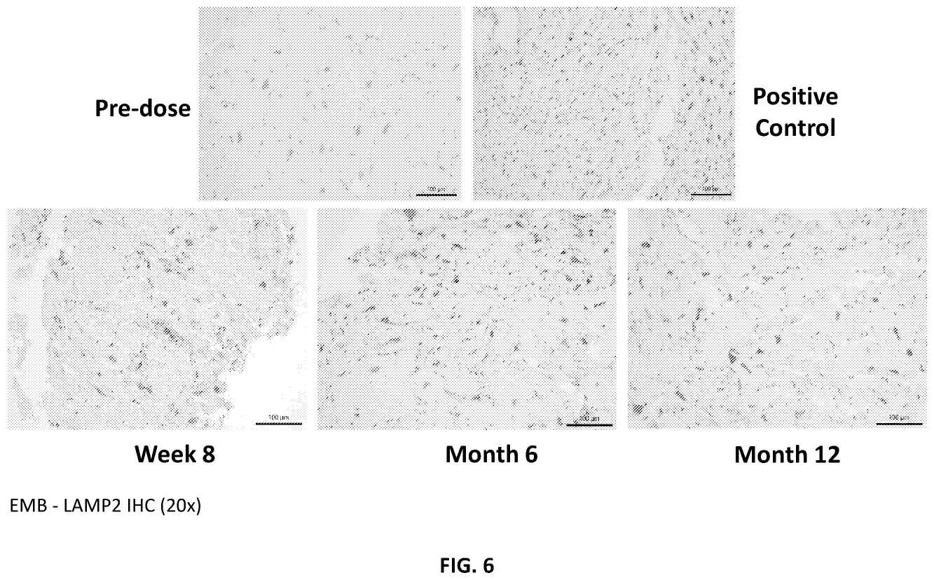

A cohort of three patients 15 years and older (Cohort 1) received LAMP-2B gene therapy at a dose of 6.7×10¹³ GC/kg with concomitant corticosteroids as per protocol. Following LAMP-2B gene therapy treatment, the vector DNA copy numbers were analyzed, as shown in FIG. 5. As shown in Table 2, all three patients demonstrated evidence of cardiac LAMP2B expression by Western Blot and immunohistochemistry, including the first patient whose compliance with the immunosuppressive regimen was limited. Patients 1002 and 1005 who had good compliance with the immunosuppressive regimen demonstrated high levels of cardiac LAMP2B expression along with clinical biomarker improvements. In cardiac biopsies of patients treated at a systemic dose of 6.7×10¹³ gc/kg, LAMP2B gene expression was demonstrated to be present in 68-92% of cells versus normal as determined by immunohistochemistry (IHC) at 9 and 12-months as well as up to 61% of normal LAMP2B protein expression that was measured by Western blot assessment in one patient. As shown in FIG. 6, patient 1002 demonstrated robust cardiac expression of LAMP2 following LAMP-2B gene therapy treatment. Patients 1002 and 1005 showed a consistent increase in percentage and level of IHC staining at the later time points.

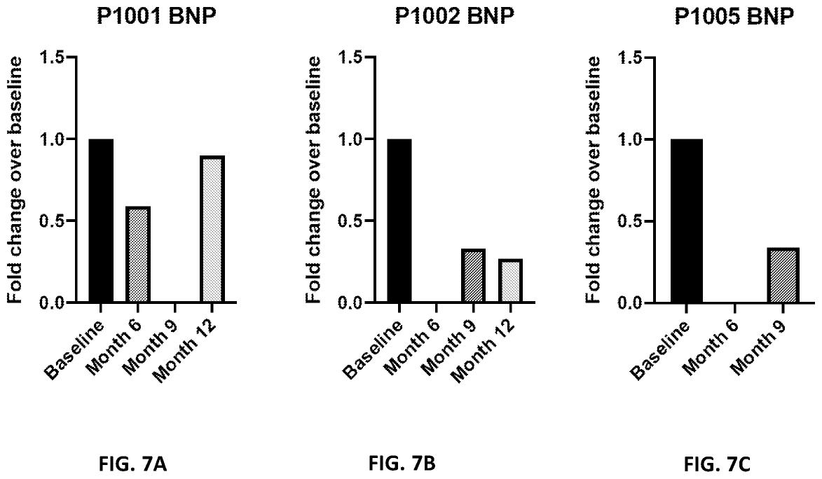

As shown in FIG. 7, two of the three patients demonstrated key clinical biomarker improvements consistent with improved cardiac function. Brain natriuretic peptide (BNP), a key marker of heart failure, improved (i.e. decreased) in all three patients, including by greater than 50% in patients 1002 and 1005 (FIGS. 7B and 7C) with confirmed immunosuppressive regimen compliance. Creatine kinase myocardial band (CPK-MB) either improved or was stabilized in patients 1002 and 1005. Notably there were visible improvements in autophagic vacuoles, a hallmark of Danon pathology, as assessed by electron microscopy. As shown in Table 3, patients 1002 and 1005 with confirmed immunosuppressive regimen compliance and follow-up at 9 and 12-months demonstrated improvement in cardiac output as measured by invasive hemodynamics. Because patients with Danon Disease do not improve independently, the benefits observed in all three patients in the present studies are clinically meaningful and offer a transformative gene therapy approach to this otherwise devastating disease.

TABLE 3

| Cardiac Output | | | | |
|---|---|---|---|---|
| (L/min)a | Baseline | Week 8 | Month 6 | Month 12 |
| Subject 1001 | 5.2 | 6.2 | 6.1 | 4.12 |
| Subject 1002 | 3.58 | 5 | ND | 5.8 |
| | | | | (1.62x increase) |
| Subject 1005 | 4.5 | 3.7 | 6.08* | ND |
| | | | (1.35x increase) | |

*Month 9

### Definition of Dose-Limiting Toxicity

Dose-limiting Toxicity (DLT) is defined as any occurrence of AE(s) occurring within 8 weeks after investigational product administration, as follows:
National Cancer Institute Common Terminology Criteria for AEs (NCI CTCAE), version 5.0, Grade 3 or higher infusion-related reactions.
Grade 4 transaminase elevations persisting for more than 2 weeks associated with liver injury despite supportive therapy.
Grade 3 transaminase elevations that do not improve or resolve with supportive regimens.
Other Grade 3 or higher AE, with the exception of Grade 3 nausea/vomiting, diarrhea, constipation, fever, fatigue, or skin rash that resolve to Grade <3 within 72 hours, or Grade 3 laboratory abnormalities not associated with clinical symptoms that resolve to Grade <3 within 2 weeks.

DLTs will be determined irrespective of Investigator-attributed causality. In settings in which there is a likely etiology not related to study therapy (for example Grade 3 pain secondary to motor vehicle accident), a determination that an event does not represent a DLT may be made but will require evaluation by the IDSMC. The baseline values for each patient will be based on available clinical data within 6 months prior to IP administration.

### Dose Escalation Procedure

Enrollment within a cohort will be staggered such that the initial patient must be followed for at least 8 weeks before subsequent patients in a cohort may receive IP. The addition of rituximab and tacrolimus is anticipated to reduce the immune response following IP administration. After the suppression of the immune response has been demonstrated, the duration of 8 weeks between subsequent patients in a cohort may be reduced with IDSMC agreement. Each cohort will consist of at least 2 patients. If 1 out of the first 2 patients in a cohort experiences a DLT, an additional 2 patients will be enrolled in the cohort. In order for activation of a subsequent (higher-dose or pediatric) cohort to commence, all patients in a prior cohort must have been followed for at least 8 weeks subsequent to infusion of IP and there has been evidence of DLT resolution or stabilization. Pediatric patients (ages 8-14) at a given dose level will commence only pending determination of safety of the dose level in cohorts 1, 2, and 3 (adults and ages 15-17) cohort.

If it is determined that administration of 6.7×10¹³ GC/kg (Low Dose) results in sub-therapeutic LAMP-2B expression with acceptable safety in an initial 2 or 3 patients in Cohort 1, Cohort 2 at an intermediate dose of 1.1×10¹⁴ GC/kg will be activated. Cohort 1A (Pediatric age 8-14) at the 6.7×10¹³ GC/kg dose will not be performed if the 6.7×10¹³ GC/kg is considered sub-therapeutic. Cohort 2A (Pediatric age 8-14) at an intermediate dose of 1.1×10¹⁴ GC/kg will commence enrollment only after completion of Cohort 2 (Adult and age 15-17) at an intermediate dose of 1.1×10¹⁴ GC/kg, and pending review of safety with IDSMC. Refer to FIG. 8.

### Investigational Product Administration

Patients will receive AAV9.LAMP2B gene therapy product via IV infusion on Day 0; it is intended that the IP will be infused as a single dose to the patient. Patients will receive investigational product in an inpatient (hospitalized) setting in a facility experienced with investigational therapeutics for cardiovascular disorders. Patients will remain hospitalized for 48-72 hours and may remain hospitalized for up to 14 days subsequent to investigational therapy infusion, and may be discharged thereafter at the discretion of the treating Study Investigator. Daily assessments will continue through Day 7 and may be extended at the discretion of the Study Investigator.

### Pre-Treatment Medications and Concomitant Treatment

Prophylaxis for anti-AAV immunogenic response will be administered prior to and following infusion of investigational product. In addition to corticosteroids, rituximab and tacrolimus will be administered to suppress the immune response to IP.

### Pre-Treatment Medications

### Pre-Medications Prior to Rituximab Infusion:

Acetaminophen—15 mg/kg PO (maximum 1 g)
Diphenhydramine—12.5 to 50 mg PO, or as per product insert
Rituximab: Patients will receive rituximab at a dose of 750 mg/m2 on Days −14 and −7 prior to IP infusion.
Corticosteroids: Patients will receive prednisone (oral or IV) at a dose of 1 mg/kg body weight starting the day prior to investigational product infusion (Day −1) and then daily through Week 8 post-treatment, followed thereafter by a 4-week taper prior to discontinuation by Month 3; in settings in which enhanced immunosuppressive therapy (i.e. rituximab, tacrolimus) is administered as prophylaxis, the corticosteroid taper may commence at an earlier juncture, including within the initial 4 weeks following IP administration, at the discretion of the Study Investigator with Sponsor's Medical Monitor.
   In settings in which prednisone results in unacceptable AEs or is unavailable, methylprednisolone or dexamethasone may be substituted at equivalent doses (0.8 mg/kg methylprednisolone; 0.15 mg/kg dexamethasone).
   The corticosteroid taper may extend over a period longer than 4 weeks in settings of increased liver enzymes (bilirubin or transaminases) over baseline or other AEs attributable to investigational product.
   The corticosteroid taper may commence earlier than Week 8 in settings of intolerable corticosteroid-related AEs or in settings of enhanced prophylactic immunosuppression as noted above, at the discretion of the Study Investigator in conjunction with the Sponsor's Medical Monitor.
   Medications to be co-administered to prevent corticosteroid complications may be given in accordance with institutional standards and should include prophylaxis for Pneumocystis jirovecii pneumonia (PCP), gastrointestinal ulcers, and constipation. If no institutional guidelines exist, then these may include and not limited to atovaquone or trimethoprim/sulfamethoxazole 3 times per week, either an H2-antagonist (i.e., ranitidine) or a proton-pump inhibitor (i.e., omeprazole), and a stool softener (i.e., docusate) with or without a laxative, administered daily at age/weight-appropriate doses.
Tacrolimus: Patients will receive tacrolimus per oral daily in 2 divided doses. Dosage will be adjusted as needed based on tacrolimus trough serum levels to maintain a level of 2-5 ng/mL, with administration starting on Day −7 through 3 months. Tacrolimus may be discontinued at the discretion of the Study Investigator.
Supportive Medications during corticosteroid course: As per institutional practice, patients will receive prophylaxis for Pneumocystis jiroveci pneumonia (PJP/PCP) prophylaxis, gastrointestinal ulcers, and constipation.
Additional pre-treatment medication(s): not required prior to infusion of investigational product but may be considered if required by institutional guidelines for infusion of AAV investigational agents; such agents may include acetaminophen (paracetamol) and/or histaminergic H1- or H2-antagonists.

### Concomitant (Supportive Care) Medications:

Additional supportive therapies may be administered, prior to or following IP administration, to prevent or treat adverse effects at the discretion of the Study Investigator. These may include and are not limited to:
Corticosteroids
Eculizumab in settings of complement activation. Prior to eculizumab administration the patient must have received meningococcal vaccination as recommended by Centers for Disease Control as appropriate for age and health condition. The meningococcal vaccination(s) may be administered by the study site prior to receiving investigational product;
Transfusion of platelets or plasma products in settings of coagulopathy;
Anti-emetics (such as ondansetron);
Growth factors (such as G-CSF [Neupogen®]) in settings of neutropenia;
Analgesic and anti-inflammatory agents in settings of infusion-related reactions

### Patient Inclusion Criteria

Patients must meet all the following criteria (and none of the exclusion criteria) to be eligible for study participation:
1. DD diagnosis with a confirmed LAMP2 mutation(s).
2. Cardiac involvement as documented by at least one abnormal finding on ECG, echocardiogram, gadolinium-enhanced cardiac MRI, or electrophysiology study.
3. Age ≥15 years for cohorts 1, 2, and 3; 8-14 years for cohorts 1A, 2A, and 3A.
4. Male gender.
5. NYHA Class II or III. Patients with NYHA Class I are eligible if unable to walk ≥450 meters during the 6MWT.
6. Adequate hematologic function as defined by:
   a. Hemoglobin ≥10 g/dL (6.2 mmol/L; Grade 1 anemia, per NCI CTCAE version 5.0).
   b. Absolute neutrophil count ≥1,500/mm3 (1.5×109/L; Grade ≤1 neutropenia).
   c. Platelet count ≥75,000/mm3 (75×109/L; Grade ≤1 thrombocytopenia).
7. Hepatic function as defined by:
   a. AST and ALT ≤10.0×ULN or GGT ≤2×ULN (transaminase elevations in DD are considered extensively to result from muscle injury; hence the relatively high upper limit for these transaminases and consideration of GGT levels, and the presence of additional hepatic eligibility markers of bilirubin and PT/INR).
   b. Serum bilirubin ≤1.5×ULN (i.e., Grade bilirubin increase).
   c. PT/INR≤1.5×ULN (in the absence of anticoagulation)
   d. Absence of cirrhosis on liver ultrasound
8. Renal function as follows: creatinine ≤1.5×ULN; (if creatinine is >1.5 ULN, then creatinine clearance ≥50 mL/min/1.73 m² is required, as calculated by Modification of Diet in Renal Disease (MDRD) equation (Stevens 2006), the revised Schwartz formula (for patients under 18 years old) (Schwartz 2009), or 24-hour urine collection).
9. Ability to provide informed consent (for adult patients and parents/legal guardians of pediatric patients) and assent (for patients age 15-17).
10. Ability to comply with study procedures including investigational therapy and follow-up evaluations.
11. Able to walk >150 meters unassisted during the 6MWT.
12. Patient has received meningococcal vaccination recommended by Centers for Disease Control as appropriate for age and health condition.

### Patient Exclusion Criteria

Patients meeting any of the following criteria are excluded from study participation:
1. IV therapy with positive inotropes, vasodilators or diuretics within the 30 days prior to enrollment (i.e., patient must be stable on oral medical therapy).
2. Prior cardiac transplantation or prior transplant of other organ (lung, liver, other).
3. Cardiac surgery, percutaneous cardiac intervention, or valvuloplasty within 30 days prior to enrollment.
4. Presence or requirement of a LVAD.
5. Myocardial infarction, unstable angina, stroke, or TIA within 90 days prior to enrollment.
6. Significant (greater than moderate) valvular stenosis or regurgitation on echocardiogram.
7. Requires mechanical ventilation.
8. Anti-AAV9 neutralizing antibody titer >1:40.
9. Concurrent enrollment in any other clinical investigation involving use of an investigational agent for the treatment of CHF or cardiomyopathy.
10. Active hepatitis B or C infection (including patients with positive HBsAg, HBeAg, or detectable HBV or HCV viral load). Patients with previous, adequately resolved HBV or HCV are eligible.
11. Significant medical conditions including documented HIV infection, active viral or other hepatitis, poorly-controlled hypertension or diabetes, poorly controlled cardiac arrhythmia, or uncontrolled viral, bacterial, or fungal infection.
12. Any concomitant medical or psychiatric condition that in the opinion of the Investigator renders the patient unfit for study participation or at higher than acceptable risk for study participation.
13. Active hematologic or solid organ malignancy, not including non-melanoma skin cancer or other carcinoma in situ. Patients with previously resected solid organ malignancies or definitively treated hematologic malignancies may be eligible if there has been no evidence of active malignancy during the prior 3 years.
14. Any contraindication to use of tacrolimus which includes hypersensitivity to tacrolimus or HC-60 (polyoxyl 60 hydrogenated castor oil).

### Study Duration

Patients will be screened and have screening assessments performed within approximately 60 days before investigational product administration on Day 0. All patients are planned to be followed for 36 months after investigational product administration under the auspices of this protocol. Overall survival will be assessed and patients who elect to discontinue other components of follow-up (strongly discouraged unless in the context of severe, prohibitive deterioration in health status); such patients will be provided the option of maintaining contact with study personnel for evaluation of overall health status and survival. After the end of the follow-up period, patients will enter a Long-Term Follow-Up (LTFU) study enabling follow-up for an additional 2 to 5 years post-IP administration.

### Long-Term Follow-Up

Following assessment of Phase 1 safety endpoints (initial 8-12 weeks post-infusion) and subsequent follow-up (initial 3 years post-infusion); ongoing follow-up for safety and toxicity (i.e., AEs), overall survival, overall health status including requirement for cardiac transplant and other adverse health outcomes will be conducted at regular intervals for an additional 2-5 years (5-8 years total, inclusive of the 3-year follow-up stipulated for the study). Long-Term Follow-Up will be planned for 5 years but will be re-evaluated if no serious AEs attributable to the investigational therapy are identified during the first 2 years.

If it is determined that no serious adverse drug reactions are identified during the study, then Long-Term Follow-Up will be planned for an additional 2 years, with longer follow-up (up to 5 years) depending on the extent, severity and resolution of AEs related to investigational product.

### Schedule of Events and Study Procedures

### Schedule of Events

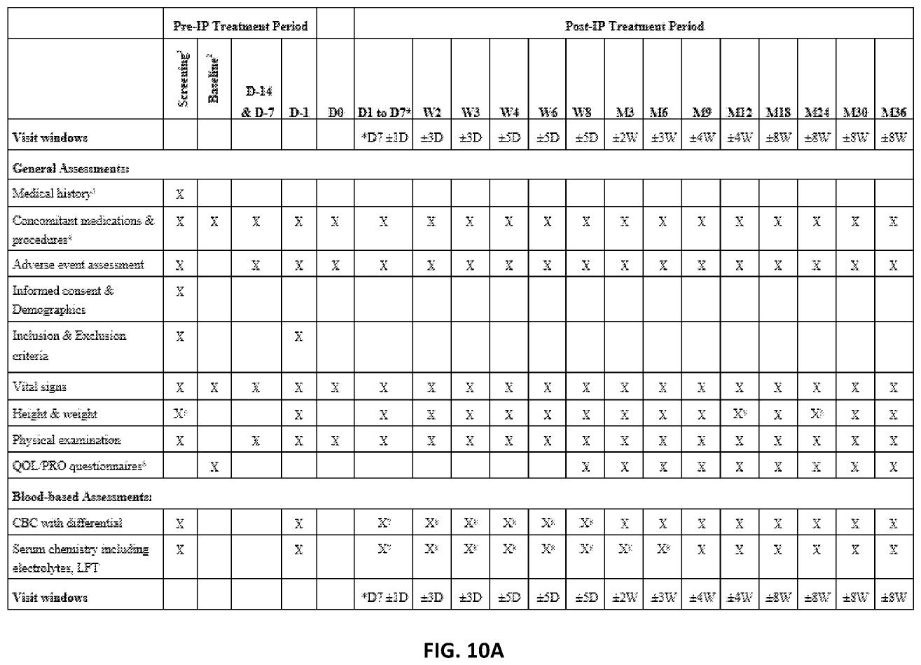

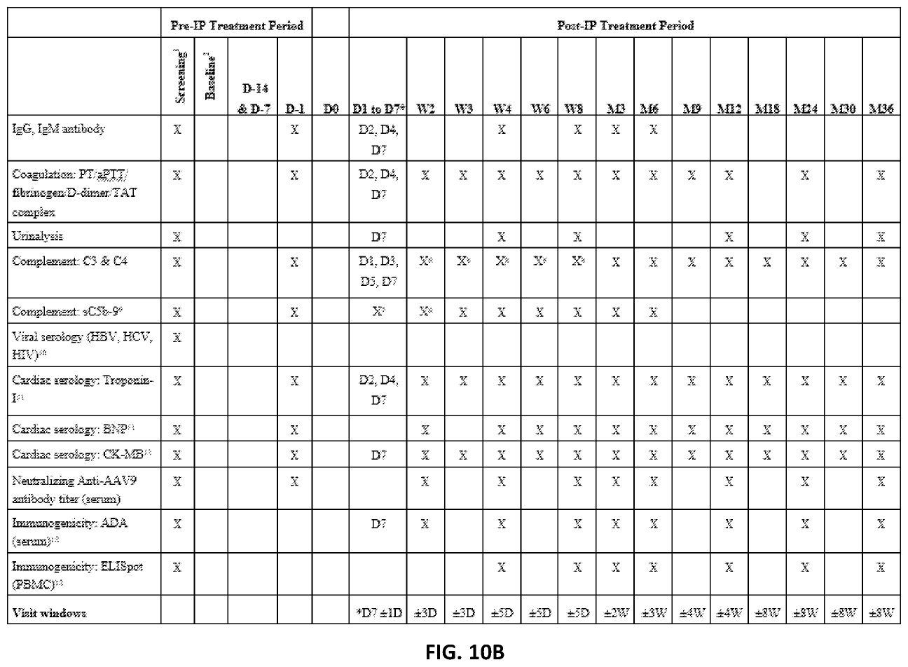

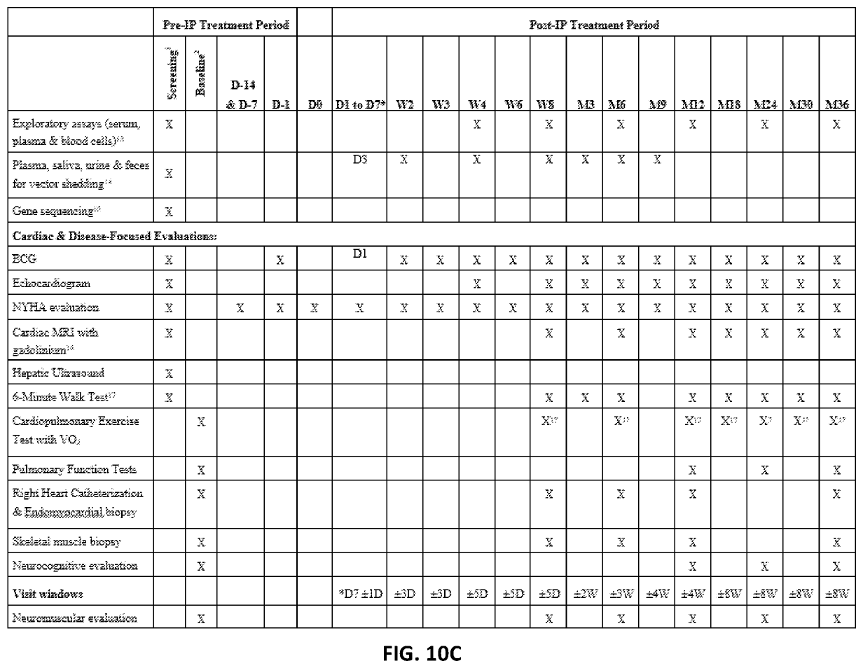

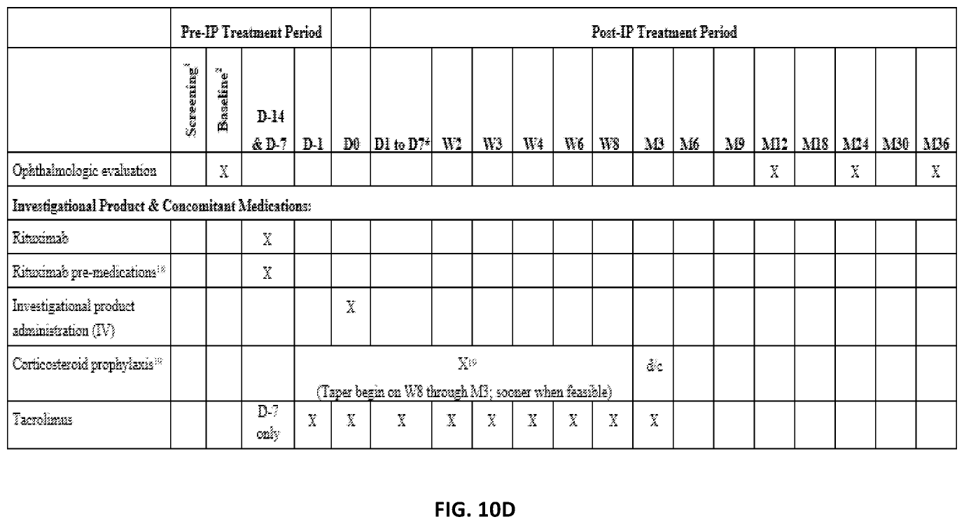

The investigational product will be administered via IV infusion on Day 0. After investigational product infusion, patients will remain hospitalized or in-patient in a dedicated research facility for at least 48-72 hours and up to 14 days after infusion at the discretion of the Study Investigator's clinical judgement. Post-IP infusion, patients should attend study visits at frequencies outlined in FIGS. 10A-10D. Assessments that are performed daily for 7 days following investigational product. Daily evaluations may continue post-Day 7.

Follow-up visits will involve a series of clinical, clinical-laboratory, cellular and genetic, cardiac imaging and cardio-physiologic evaluations occurring in the days, weeks, months and years following infusion of investigational product. An endomyocardial biopsy (performed via central venous access catheterization of the right ventricle) and a skeletal muscle biopsy will also be required prior to, and at selected time points subsequent to investigational product infusion.

Follow-up regarding evidence LAMP2B gene and protein in cardiomyocytes (and histologic evaluation of DD histology including quantification of autophagic vacuoles) will occur via endomyocardial biopsy 1 at Week 8 and Months 6, 12, and 36 post-infusion. As detailed subsequently, additional less invasive assessments, including skeletal muscle biopsy, evaluation of LAMP2B blood levels (plasma (LAMP-2B protein) and mononuclear cells (LAMP2B DNA) and other serologic and radiographic parameters of cardiomyopathy and CHF, will be assessed concomitantly with the exploratory intent of identifying potential surrogate markers of molecular and histologic improvement in myocardium. The detailed Schedule of Events is presented in FIGS. 10A-10D, footnotes to which are as follows:
1. Screening visit assessments will be performed over a 60-day period. Assessments performed as part of standard of care prior to enrollment may be used for the study as screening assessments and do not need to be repeated (based on Study Investigator judgement in consultation with the Sponsor).
2. Baseline visit assessments may be performed after patient has completed the screening visit and have met all inclusion and exclusion criteria. All assessments must be completed by Day −1 and prior to the administration of investigational product. Baseline visit assessments do not need to be completed before rituximab and tacrolimus treatment has started.
3. During the screening visit, a comprehensive medical history will include any relevant cardiovascular and other potential disease-related prior events, including hospitalizations and cardiac interventions; family history of disease will also be recorded.
4. Concomitant medications and procedures during the screening visit and Day −1 should include any medications & procedures during the prior 30 days. During the follow-up visits, this should include any medications and procedures subsequent to the most recent prior evaluations.
5. Height will be collected once at the screening visit and annually thereafter.
6. Quality of Life/Patient Reported Outcomes questionnaire will include the KCCQ-12 and PedsQL.
7. Daily laboratory assessments Day 1 through Day 7.8. Laboratory assessments will be performed up to 3 times per week.
9. Serum membrane attack complex (sC5b-9) is required at the protocol specified time points post-administration of the investigational product. The test may be performed at additional timepoints or more frequently depending on the clinical status of the patient, and at the discretion of the Investigator.
10. Viral serology during the screening visit should include HIV, HBV and HCV viral load, and comprehensive HBV antigen/antibody panel including HBsAg, HBeAg, and HBsAb.
11. Cardiac serology will include potential markers of CHF including BNP, CK-MB, and Troponin-I levels.
12. Immunologic response to the investigational product (AAV9 and LAMP-2B) will include evaluations in serum (anti-drug antibody (ADA); IgG, IgM) and in peripheral blood T lymphocytes (ELISpot).
13. Whole blood will be collected, processed for serum, plasma and PBMCs (for screening assessment only) and stored for potential exploratory assays, including evaluation of LAMP2 levels in serum and blood cells. The study ICF will provide patients the opportunity to provide or withhold consent that stored samples may be utilized for potential additional future research not specified in the protocol.
14. A screening saliva, urine, feces, and blood (plasma) sample will be collected subsequent to investigational product infusion for the evaluation of vector particle shedding. Evaluation of each bodily fluid/substance will continue as indicated until there are two negative evaluations (or when the vector levels have plateaued at low or negligible levels) for a given fluid/substance, at which point no subsequent evaluations are required.
15. Gene sequencing to confirm and define LAMP2 mutations will be conducted by means of an Arrhythmia and Cardiomyopathy Panel via either saliva or blood (mononuclear cells). If the LAMP2 genotype is not defined as pathogenic, a protein assessment for LAMP2 will be performed from isolated peripheral blood mononuclear cells or biopsy tissue to confirm LAMP2 deficiency.
16. MRI with gadolinium will be performed when not contraindicated by presence of implanted pacemakers, defibrillators, other indwelling devices or medical conditions (i.e., renal dysfunction precluding the use of gadolinium contrast, per institutional guidelines). In settings of renal dysfunction, non-gadolinium contrast agents may be utilized at the discretion of the consulting radiologist or in accordance with institutional guidelines. In settings where MRI is contraindicated, CT scan may be used.
17. When feasible, 6-minute walk should be performed at a similar time during the day for the initial and subsequent evaluations. During follow-up assessments, the 6-minute walk and cardiopulmonary exercise tests should, whenever possible, not be performed on the same day.
18. Rituximab pre-medications, acetaminophen and diphenhydramine, will be given 30-60 minutes prior to Rituximab infusion. Refer to the Pharmacy Manual for details.
19. Corticosteroids will be administered daily starting on Day −1 at a dose of prednisone (IV or PO) 1 mg/kg (or methylprednisolone or dexamethasone equivalent) through W8. Corticosteroid taper will commence on W8 (Day 56) following investigational product, with discontinuation (d/c) planned by 3 months following investigational product. A more protracted taper, or initiation of taper at a time point prior to or subsequent to Day 56 is permitted at the discretion of the treating Investigator in conjunction with the study medical monitor. Supportive medications for corticosteroid administration may be provided per institutional standards.

### Neutralizing Anti-AAV9 Antibody Titers in Serum

A blood sample for determination of neutralizing anti-AAV9 antibody titer in serum is to be collected at screening; Day −1, Week 2, Week 4, Week 8, Month 3, Month 6, Month 12, Month 24, Month 36 as per Schedule of Events (FIGS. 10A-10D). Patients with anti-AAV9 neutralizing antibody titers >1:40 are not eligible for study participation.

### Hepatic Ultrasound

In addition to laboratory assessment of bilirubin, PT/INR and transaminase levels, an ultrasound of the liver will be performed at screening to evaluate findings consistent with cirrhosis or other hepatic compromise. Subsequent (post-infusion) ultrasounds may be performed if clinically indicated.

### Safety Assessments

### Vital Signs

Vital signs to be measured include systolic/diastolic blood pressure, pulse, respiration rate, pulse oximetry, and temperature, and will be performed in accordance with institutional standards. Vital signs will be measured at every study visit as per the Schedule of Events (FIGS. 10A-10D).

### Height and Weight

Height (cm) will be measured at screening and annually thereafter. Weight (kg) will be measured at screening; Day −1; once a week from Week 1 through Week 8; once every 3 months (Month 3-Month 12); and once every 6 months (Month 12-Month 36) as per the Schedule of Events (FIGS. 10A-10D). The weight measurement taken on Day −1 will be used in the calculation of the patient's dose.

### Clinical and Physical Examination

A complete physical examination (including performance status, general appearance; head eyes, ears, nose, and throat; cardiovascular; dermatologic, abdominal; genitourinary; lymph nodes; hepatic; musculoskeletal; respiratory; and neurological) is to be conducted screening; Day −14 and Day −7; Day −1; Day 0; daily Day 1 through Day 7; once a week from Week 2 through Week 8; once every 3 months (Month 3-Month 12); and once every 6 months (Month 12-Month 36) as per the Schedule of Events (FIGS. 10A-10D).

### Hematology, Coagulation Studies, and Chemistry

Blood samples for clinical laboratory tests, including CBC and differential, coagulation studies, and chemistry, will be performed as specified below, and in the Schedule of Events (FIGS. 10A-10D). Clinical laboratory tests are to be performed and reviewed by the Investigator or qualified designee (e.g., physician's assistant, nurse practitioner). The following clinical laboratory parameters are to be determined:
Hematology: Hemoglobin, hematocrit, erythrocyte (RBC) count, mean cell volume (MCV), mean cell hemoglobin concentration (MVHC), platelets, white blood cell (WBC) count and differential including neutrophils, lymphocytes, monocytes, eosinophils, and basophils.
   Chemistry: Sodium, potassium, chloride, carbon dioxide (or bicarbonate), blood urea nitrogen (BUN), creatinine, glucose, ALP, ALT, AST, bilirubin, GGT, calcium, magnesium, and phosphorus.

Hematology and chemistry evaluations will be performed at screening; Day −1; daily Day 1 through Day 7; up to 3× per week from Week 2 through Week 4 (hematology), through Week 8 (chemistry); once every 3 months (Month 3-Month 12); and once every 6 months (Month 12-Month 36). During the screening visit if serum creatinine is >1.5×ULN, then creatinine clearance (CrCl) may be calculated, and the patient will be considered eligible if CrCl>50 mL/min/1.73 m², as calculated by MDRD equation.
Coagulation Studies: PT (and/or INR), aPTT, D-dimer, thrombin-antithrombin complex (TAT), and fibrinogen. Evaluations will be performed at screening; Day −1, Day 2, Day 4, Day 7; once a week from Week 2 through Week 8; once every 3 months (Month 3-Month 12); and once every 6 months (Month 12-Month 36).
Complement: C3, C4, and sC5b-9 membrane attack complex.

C3 and C4 evaluations will be performed at screening; Day −1, Day 1, Day 3, Day 5, Day 7; up to 3× per week from Week 2 through Week 8; once every 3 months (Month 3-Month 12); and once every 6 months (Month 12-Month 36). sC5b-9 evaluations will be performed at screening; Day −1; daily Day 1 through Day 7; up to 3× per week during Week 2; once a day from Week 3 through Week 8; Month 3 and Month 6. Additional clinical laboratory tests may be performed at the Study Investigator's discretion.

### Cardiac Serology

Blood samples for cardiac serology are to be collected per the Schedule of Events (FIGS. 10A-10D). Parameters to be measured include:
BNP collected at screening; Day −1, once during Week 2, Week 4, Week 6, Week 8; once every 3 months (Month 3-Month 12); and once every 6 months (Month 12-Month 36)
CK-MB collected at screening; Day −1, Day 7, once a week from Week 2 through Week 8; once every 3 months (Month 3-Month 12); and once every 6 months (Month 12-Month 36)
High-sensitivity troponin levels collected at screening; Day −1, Day 2, Day 4, Day 7; once a week from Week 2 through Week 8; once every 3 months (Month 3-Month 12); and once every 6 months (Month 12-Month 36)

### Urinalysis

Urine sample for specific gravity, pH, protein, glucose, ketones, blood, urine leukocyte esterase will be collected at screening; Day 7, Week 4, Week 8; once during Month 12, Month 24, and Month 36 as per the Schedule of Events (FIGS. 10A-10D).

### Immunogenicity

Blood samples for immunogenicity are to be collected for determination of humoral (antibody) and cellular (T-lymphocyte) anti-AAV9 and anti-LAMP-2B protein activity in whole blood and serum; IgG and IgM will also be collected as per the Schedule of Events (FIGS. 10A-10D). Blood samples for antibody evaluation will be collected at screening; Day 7; once during Week 2, Week 4, Week 8, Month 3, Month 6, Month 12, Month 24, and Month 36. Blood samples for cellular evaluation will be collected at screening; once during Week 4, Week 8, Month 3, Month 6, Month 12, Month 24, and Month 36. Blood samples for IgG and IgM evaluation will be collected at screening; Day −1, Day 2, Day 4, Day 7; once during Week 4, Week 8, Month 3, and Month 6.

### Vector Shedding

Blood (plasma), saliva, urine, and fecal samples will be collected at screening; Day 3, once during Week 2, Week 4, Week 8, Month 3, Month 6, and Month 9 according to the Schedule of Events (FIGS. 10A-10D) for evaluation of vector particle shedding. Evaluation of each bodily fluid/substance will continue as indicated until there are two negative evaluations or when the vector levels have plateaued at low or negligible levels for a given fluid/substance, at which point no subsequent evaluations are required.

### Adverse Events

All AEs occurring from provision of informed consent and, if applicable, assent will be recorded. This includes AEs the patients report spontaneously, those observed by the Investigator, and those elicited by the Investigator in response to open-ended questions during scheduled study center visits. Information to be systematically recorded incudes the type of AE, dates of onset and resolution, severity, and perceived relationship to experimental therapy. The severity of each AE will be rated based on the NCI CTCAE, version 5.0.

### Efficacy Assessments

### Electrocardiogram

Twelve-lead ECGs are to be performed at screening; Day −1; Day 1; once a week from Week 2 through Week 8; once every 3 months (Month 3-Month 12); and once every 6 months (Month 12-Month 36) as per the Schedule of Events (FIGS. 10A-10D).

### Echocardiogram

Echocardiography is to be performed at screening; Week 4, Week 8; once every 3 months (Month 3-Month 12); and once every 6 months (Month 12-Month 36) as per the Schedule of Events (FIGS. 10A-10D). Components of the echocardiogram include assessment of Left Ventricular Ejection Fraction (LVEF), Left and Right Ventricular Dimensions (for example, LV end-systolic and end-diastolic dimensions, volumes and indices, septal and posterior wall thickness and LV outflow tract dimension), assessments of concentric hypertrophy and diastolic patterns, assessments of valvular stenosis and regurgitation, assessments of wall motion abnormalities, pulmonary pressures, IVC size and changes with respiration, pericardium, LV mass, Left Atrial (LA) diameter and volume, Isovolumetric relaxation time, Doppler velocity measurements, global longitudinal strain, and Left Ventricular Outflow Tract (LVOT) grading.

### New York Heart Association (NYHA) Classification

The NYHA classification provides a simple way of classifying the extent of heart failure according to the severity of symptoms, as shown in Table 4. It places patients in one of the four categories based on how much they are limited during physical activity. NYHA evaluation will be performed at every study visit, with the exception of the baseline visit as per the Schedule of Events (FIGS. 10A-10D).

TABLE 4

| Classification | Patient Symptoms |
|---|---|
| I | No limitation of physical activity. Ordinary physical activity does not cause |
| | undue fatigue, palpitations, dyspnea (shortness of breath). |
| II | Slight limitation of physical activity. Comfortable at rest. Ordinary physical |
| | activity results in fatigue, palpitations, dyspnea (shortness of breath). |
| III | Marked limitation of physical activity. Comfortable at rest. Less than ordinary |
| | activity causes fatigue, palpitation, or dyspnea. |
| IV | Unable to carry on any physical activity without discomfort. Symptoms of heart |
| | failure at rest. If any physical activity is undertaken, discomfort increases. |

### Cardiac Magnetic Resonance Imaging

MRI with gadolinium will be performed when not contraindicated by presence of implanted pacemakers, defibrillators, other indwelling devices or medical conditions (i.e., renal dysfunction precluding the use of gadolinium contrast, per institutional guidelines). In settings of renal dysfunction, non-gadolinium contrast agents (i.e., ferumoxytol) may be utilized at the discretion of the consulting radiologist or in accordance with institutional guidelines. Evaluations will be performed during screening; Week 8, Month 6, Month 12, Month 18, Month 24, Month 30, and Month 36 as per the Schedule of Events (FIGS. 10A-10D).

Cardiac MRI will involve injection of IV gadolinium and acquisition of multiple sequences over an estimated 30-40-minute total scan time. Assessments will include LVEF, LV mass indexed for body surface area (BSA), Maximal LV wall thickness, z Score for maximal wall thickness, LV end-diastolic and end-systolic volumes, LA diameter, volume and volume index, assessment of Late Gadolinium Enhancement (LGE), and LGE patterns (Raja 2018). Additional assessments will include resting perfusion myocardial blood flow (MBF), extracellular volume (T1 map pre- and post-gadolinium), and Right Ventricular Ejection Fraction (RVEF). In settings where MM is contraindicated, cardiac CT scan with intravenous contrast may be used instead of MM.

### Six-Minute Walk Test

The 6MWT is a practical and simple test that requires a 100-ft hallway but no exercise equipment or advanced training for technicians. This test measures the distance that a patient can quickly walk on a flat, hard surface in a period of 6 minutes, and thereby is a quantitative assessment of an important day-to-day activity that is progressively compromised in patients with DD. During the screening visit, patients must be able to walk >150 meters unassisted during the 6MWT to be eligible for study participation. Additionally, Ross Class I patients will be considered eligible if they are unable to walk at least 450 meters unassisted during the 6MWT.

The 6MWT is to be completed at screening; Week 8, Month 3, Month 6, Month 12, Month 18, Month 24, Month 30, and Month 36 as per the Schedule of Events (FIGS. 10A-Whenever possible should be performed at the same time of the day at each timepoint evaluated during study follow-up. Identical instructions at specified intervals during the 6MWT will be given to each patient at each timepoint when the evaluation is conducted. The 6MWT should be performed on a different day than the CPET at timepoints when both evaluations are stipulated. This assessment will be performed twice at the time of each study visit, approximately 24 hours apart.

### Cardiopulmonary Exercise Testing

Cardiopulmonary Exercise Testing (CPET), including assessment of oxygen consumption (VO₂), is to be performed at baseline; Week 8; Month 6, Month 12, Month 18, Month 24, Month 30, and Month 36 as per the Schedule of Events (FIGS. 10A-10D). CPET involves evaluations on a cycle ergometer or treadmill including resting, unloaded exercise and incremental ramp exercise designed to yield 8-12 minutes of total exercise duration with measurement of expired gases for determination of oxygen consumption and carbon dioxide production. Measurements include vital signs, pulmonary indices (including maximum voluntary ventilation), ventilatory threshold measurements (including respiratory exchange ratio and the ratio of minute ventilation to carbon dioxide production), peak exercise measurements (including peak vital signs and peak VO₂), anaerobic threshold measurements (including VO₂) and recovery assessments. The CPET should be performed on a different day than the 6MWT at each timepoint when both evaluations are stipulated.

### Pulmonary Function Testing

Pulmonary Function Testing (PFTs) will be evaluated at baseline; Month 12, Month 24, Month 36 as per the Schedule of Events (FIGS. 10A-10D) to enable assessment of both pulmonary and diaphragmatic muscle capacity. PFT evaluations will include measurements of flow volume (including FVC, FEV₁), vital capacity, and diffusion capacity (including DLCO₂). Maximal inspiratory and expiratory pressures will also be evaluated.

### Right Heart Catheterization and Endomyocardial Biopsy

Right heart catheterization and endomyocardial biopsy will be performed at baseline; Week 8, Month 6, Month 12, and Month 36 as per the Schedule of Events (FIGS. 10A-10D). The catheterization and biopsy will be performed by an interventional cardiology team with expertise in this procedure. The use of anesthesia during the right heart catheterization and endomyocardial biopsy procedure will be based on Institutional guidelines and per the Study Investigator's clinical judgement. Risks associated with anesthesia are described in product package inserts.

Right Heart Catheterization with Hemodynamic Assessment

A right heart catheterization will be performed to enable assessment of cardiopulmonary hemodynamic parameters. Hemodynamic parameters assessed will include right atrial and pulmonary artery pressures, pulmonary artery wedge pressure, mixed venous oxygen saturation, and assessments of cardiac output and cardiac index (Fick formula), pulmonary capillary wedge pressure, pulmonary vascular resistance, and pulmonary hypertension.

### Endomyocardial Biopsy

The endomyocardial biopsy will be performed via central venous access catheterization of the right ventricle and catheter-mediated biopsy of the intraventricular septum, involving approximately 3-5 samples per procedure.

The biopsy will enable evaluation of any therapy-related alterations in LAMP2B gene/protein expression and changes in DD-related myocardial histology (i.e., autophagic vacuoles, myofibrillar disarray). The recommendations for right heart catheterization and endomyocardial biopsy during subsequent investigations will be evaluated based on the extent of observed histologic changes and LAMP-2B expression during Phase 1 and including potential correlation of myocardial molecular and histologic changes with improvements in parameters from less invasive assessments, including LAMP-2B expression from skeletal muscle biopsy, LAMP-2B levels in blood, and other serologic and radiographic parameters of cardiomyopathy and CHF.

### Skeletal Muscle Biopsy

Skeletal muscle biopsies will be performed to evaluate LAMP2B gene/protein expression in skeletal muscle, both in order to ascertain the potential of the investigational product to prevent or reverse the musculoskeletal components of DD, and to enable assessment as to whether a LAMP2 skeletal muscle is a potential viable surrogate of LAMP2 myocardial genetic correction and protein expression. An open biopsy of the Vastus Lateralis Muscle will be performed at baseline; Week 8, Month 6, Month 12, and Month 36 to enable assessment of the parameters detailed above. Biopsies at sequential assessments (i.e., baseline and 8 weeks post-therapy) will alternate between contralateral muscles (i.e., right leg at baseline, left leg at post-therapy assessment) in order to minimize potential procedure-associated side-effects. In subsequent (Phase 2) studies, the requirement for muscle biopsy will be evaluated based on the extent of observed histologic changes and LAMP-2B expression during Phase 1 study, and correlation between endomyocardial and skeletal muscle gene/protein expression. Consideration will also be given to utilization of a needle biopsy during subsequent studies. The use of anesthesia during skeletal muscle biopsy procedures will be based on Institutional guidelines and per the Study Investigator's clinical judgement. Risks associated with anesthesia are described in product package inserts. The skeletal muscle biopsy may be performed in conjunction with the endomyocardial biopsy to limit anesthesia or sedation if needed.

### Neurocognitive Evaluation

For patients aged ≥16 years, and considered to possess normal or near-normal (mildly limited) cognitive function, the neurocognitive evaluation will include the following components:
Wechsler Adult Intelligence Scale (WAIS-IV).
Vineland Adaptive Behavior Scale, Third Edition (Vineland-3).

Patients aged ≥16 years with more limited cognitive function will not be evaluated by means of the WAIS-IV. These patients will be evaluated by the following components:
Vineland Adaptive Behavior Scale, Third Edition (Vineland-3).
Differential Ability Scales (DAS-II).

The determination of the most appropriate evaluation will be made by the evaluating psychologist/neurocognitive assessment expert, in conjunction with the Study Investigator.

For patients aged <18 years, the neurocognitive evaluation will include the following components:
WAIS-IV (if age 16 or 17 and considered of normal or near-normal cognitive function).
Vineland-3 Parent/Caregiver assessment.
Differential Ability Scales (DAS-II).
   Neurocognitive assessments are evaluated at baseline; Month 12, Month 24, and Month 36 as per the Schedule of Events (FIGS. 10A-10D). These should be undertaken in the same sequence at each assessment whenever possible. Brief descriptions of each instrument are provided below.

### Wechsler Adult Intelligence Scale (WAIS-IV)

The WAIS-IV provides a brief, reliable measure of cognitive ability. Contains sets of standardized questions and tasks for assessing an individual's potential for purposeful and useful behavior. Designed to measure major mental abilities. This test yields standardized scores of an overall estimate of general cognitive ability, verbal comprehension, and nonverbal abilities.

### Vineland Adaptive Behavior Scale, Third Edition (Vineland-3)

The Vineland-3 is a standardized measure of adaptive behavior—the things that people do to function in their everyday lives. Whereas ability measures focus on what the examinee can do in a testing situation, the Vineland-3 focuses on what he or she actually does in daily life. Because it is a norm-based instrument, the examinee's adaptive functioning is compared to that of others his or her age. This is the leading instrument for supporting the diagnosis of intellectual and developmental disabilities. The Vineland-3 Parent/Caregiver Interview Form measures adaptive behavior functioning across 4 domains: communication, daily living, socialization, and motor functioning in individuals (birth through 90 years).

### Differential Ability Scales (DAS-II)

The DAS-II is a comprehensive, individually administered, clinical instrument for assessing the cognitive abilities that are important to learning. The test may be administered to children ages 2 years 6 months (2:6) through 17 years 11 months (17:11) across a broad range of developmental levels. This test yields standardized scores for overall general conceptual ability, verbal ability (verbal concepts and knowledge), nonverbal ability (complex, nonverbal, inductive reasoning requiring mental processing), and spatial ability (complex visual processing).

### Neuromuscular Evaluation

The neuromuscular evaluation will be performed at baseline; Week 8, Month 6, Month 12, Month 24, and Month 36. It will include timed tests of essential neuromuscular activities, including the following:
Rise from floor,
4-stair climb and descend,
10-meter walk/run,
Timed up-and-go (TUG), and
North-Star Ambulatory Assessment Test.

### Ophthalmologic Examination

Ophthalmologic examinations will be evaluated at baseline; Month 12, Month 24, and Month 36 as per the Schedule of Events (FIGS. 10A-10D). It will include retinal evaluation by direct ophthalmoscopy/fundoscopy, fundus photography, optical coherence, tomography, autofluorescence testing, and electroretinography.

### Patient-Reported Outcomes/Quality-of-Life (PRO/QOL)

PRO/QOL measures to be employed in this study include the KCCQ-12 and PedsQL. Assessments will be collected at baseline; Week 8, once every 3 months (Month 3-Month 12); and once every 6 months (Month 12-Month 36) as per the Schedule of Events (FIGS. 10A-10D).

### Efficacy Assessment

Assessments for clinical efficacy endpoints will include the following assessments performed as per the Schedule of Events (FIGS. 10A-10D) during the initial months and year following investigational product administration:
CPET, including assessment of VO2.
6MWT (distance) assessment.
Endomyocardial biopsy (performed via central venous access catheterization of the right ventricle) for evaluation of DD-associated histologic abnormalities and presence of LAMP2B gene/RNA/protein in cardiomyocytes.
Gadolinium-enhanced cardiac MRI (when not contraindicated by presence of implanted pacemakers, defibrillators, other indwelling devices, or medical conditions).
Echocardiogram.
Overall survival will be assessed and patients who elect to discontinue other components of follow-up (strongly discouraged unless in the context of severe, prohibitive deterioration in health status); such patients will be provided the option of maintaining contact with study personnel for evaluation of overall health status and survival.
Evaluation of requirement for subsequent cardiac transplantation, LVAD, implantable cardioverter-defibrillator or pacemaker placement, electrophysiologic ablative procedure for cardiac conduction aberrancy or hospitalizations for CHF.

### Statistical Methods

A sample size of up to 3 patients for a dosing cohort, with expansion up to 6 patients (in the event of a DLT in 1 of 3 patients) is considered a standard and safe approach regarding dose-evaluation of a novel investigational therapeutic. Assuming a true DLT rate of 5% or less, there would be a 3% chance that dose escalation would be halted based on a given cohort (i.e., observation of 2 or more patients with DLT). If a true DLT rate of 50% is assumed, then there would be an 83% chance that dose escalation would be halted based on a given cohort.

### Number of Patients Planned to be Enrolled

11-23 (Cohort 1 enrolled 3 patients per previous protocol version)

### Study Populations

Study populations evaluated will include the overall study population, the study populations will receive a range of investigational product. Additional evaluated populations will be adult patients (both age 18 and over and including patients age 15-17) and pediatric populations including the population age 8-14.

### Example 6: Phase 1 Study Results

Phase 1 study results have demonstrated strong trends in many key clinical biomarkers and endpoints.

DD is a genetically inherited cardiomyopathy, the features and progression of disease are distinct from those of a typical adult cardiomyopathy. The majority of patients are well compensated with respect to functional impairment until late in the disease course; measurements such as LVEF and even 6MWT may be normal or only mildly abnormal until the cardiomyopathy has progressed. Mild improvement or stabilization of disease-related abnormalities are desirable in this patient population and are likely to represent an improvement over the emerging natural history; these may be accompanied by stabilization and/or improvement in clinical biomarkers that have been shown to correlate with progression in natural history studies.

The clinical data in both low and high dose adult and adolescent cohorts ages >15 yo (6.7×10¹³ GC/kg and 1.1×10¹⁴ GC/kg) have demonstrated efficacy of treatment. The data include:
1. Confirmation of durable protein expression of LAMP2 in the primary target organ.
2. Demonstration at the cellular level of improvement in morphologic DD hallmarks.
3. Improvement and/or stabilization of key cardiac parameters including BNP (decreases in each of the 4 patients with long term follow-up, including 75%-79% decreases from baseline in the 2 low dose patients for whom there was confirmed compliance with the immunosuppressive regimen).
4. Improvement in the NYHA Class in 3 of 4 adults with confirmed immunosuppression in the low and high dose cohort and stabilization in the other low dose patient, and stabilization to mild improvements in 6MWT in each of the 4 patients
5. Self-reported improvement in quality of life and function in all low dose-treated subjects.

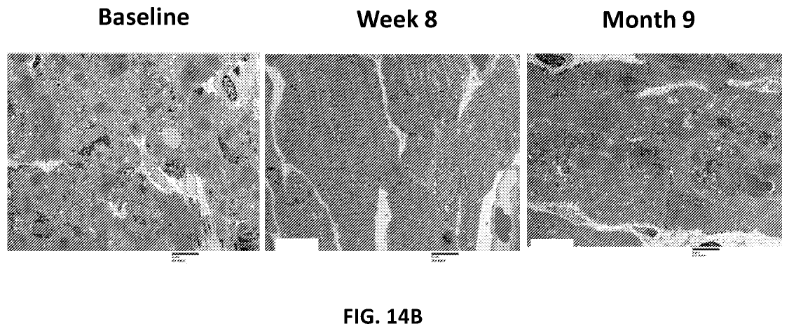

Stable LAMP2B expression data (FIG. 14) has been accompanied by demonstrable and favorable changes in the myocardial architecture, and resolution of the pathologic DD hallmarks, as assessed via electron microscopy of endomyocardial biopsy samples from pre- and post-treatment timepoints. These are illustrated FIG. 14 depicting myocardial tissue from Subject 1005. Resemblant of the findings in the murine knockout model, the pre-treatment biopsy indicates numerous and widespread autophagic vacuoles and profound derangement of the cardiac myofibrils such that distinct muscle elements are minimally discernable. A similar endomyocardial biopsy at 8 weeks post-treatment demonstrates marked diminution of the autophagic vacuoles, and restoration of myofibrillar architecture with widespread evident striation. These findings are confirmed at the later 9 month timepoint, suggesting that the molecular and histologic resolution is sustained.

Further data regarding relevant cardiac clinical markers in the low dose cohort also demonstrate either improvement or stabilization of important markers of cardiac function including B-type natriuretic peptide (BNP) (Table 5), creatine kinase-myocardial band(MB), and cardiac output as measured by invasive hemodynamics Importantly, the observed improvements in BNP from baseline (1002, 79% and 1005, 75% at 18 and 15 months respectively) were considered highly relevant and promising by experts in the field as natriuretic peptides strongly correlate with prognosis in heart failure (Januzzi et al. J Investig Med. 2013 August; 61(6): 950-955).

One of the most meaningful parameters of clinical benefit is a patient's level of function, and specifically within the context of heart failure, ability to tolerate physical activity as part of daily life. Endpoints such as New York Heart Association(NYHA) classification are therefore considered a strong measure of clinical improvement that is linked to global patient functional status (Russel et al. Am Heart J. 2009 October; 158(4 Suppl): S24-S30.). It is significant and highly inconsistent with the natural history of DD that 3 of the 4 subjects with long term follow up in the low and high dose cohort have demonstrated improvement in NYHA classification from II to I (Table 5) and the fourth subject has remained at a stable level of II.

As of the most recent long-term assessment in November 2021, all three patients have demonstrated stabilization and/or slight improvement in their 6MWT results (Table 5). In addition, as reported from the PI interview at the most recent visits, all subjects report feeling generally well and do not report having limitations in their activities. Subjects 1002 and 1005 also have reported that they have not had any firing of their implantable cardiac defibrillators since receiving RP-A501.

TABLE 5
Phase 1 Clinical Endpoints in the Low and High Dose Cohort

| | Patient | | | Most Recent | Time of |
|---|---|---|---|---|---|
| Cohort | ID | Variable | Baseline | Follow-up | Follow-up |
| Adult- | 1001* | NYHA | II | II | 24 months |
| Low | | class | | | |
| Dose | | BNP | 70 | 30 | |
| | | (pg/mL) | | | |
| | | 6 MWT | 443 | 467 | |
| | | (meters) | | | |
| | 1002 | NYHA | II | I | 18 months |
| | | class | | | |
| | | BNP | 942 | 200 | |
| | | (pg/mL) | | | |
| | | 6 MWT | 405 | 410 | |
| | | (meters) | | | |
| | 1005 | NYHA | II | I | 15 months |
| | | class | | | |
| | | BNP | 176 | 44 | |
| | | (pg/mL) | | | |
| | | 6 MWT | 427 | 435 | |
| | | (meters) | | | |
| Adult- | 1006 | NYHA | II | I | 12 months |
| High | | class | | | |
| Dose | | BNP | 123 | 41 | |
| | | (pg/mL) | | | |
| | | 6 MWT | 436 | 492 | |
| | | (meters) | | | |

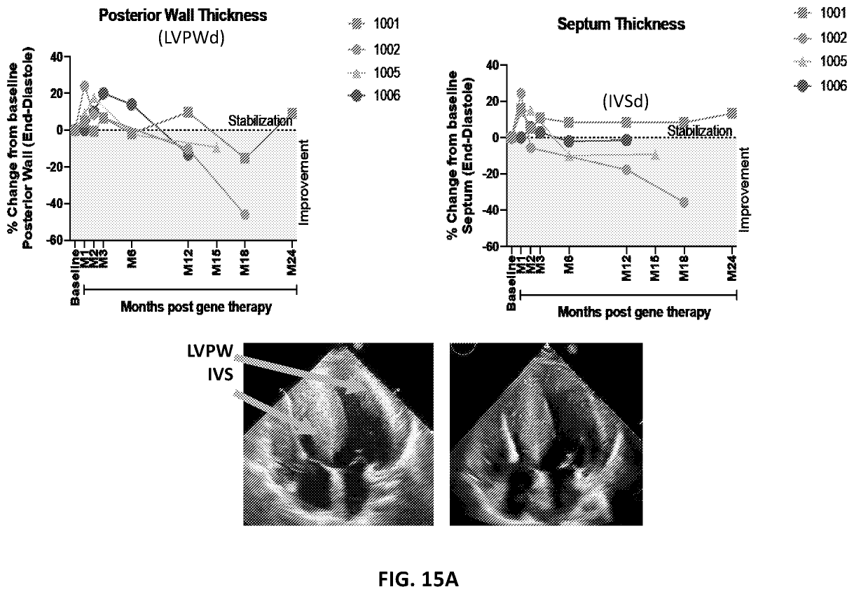

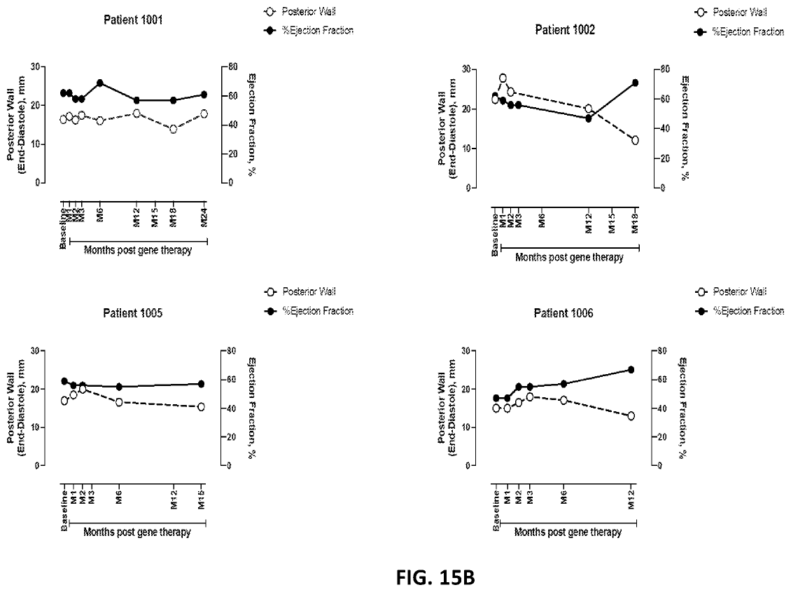

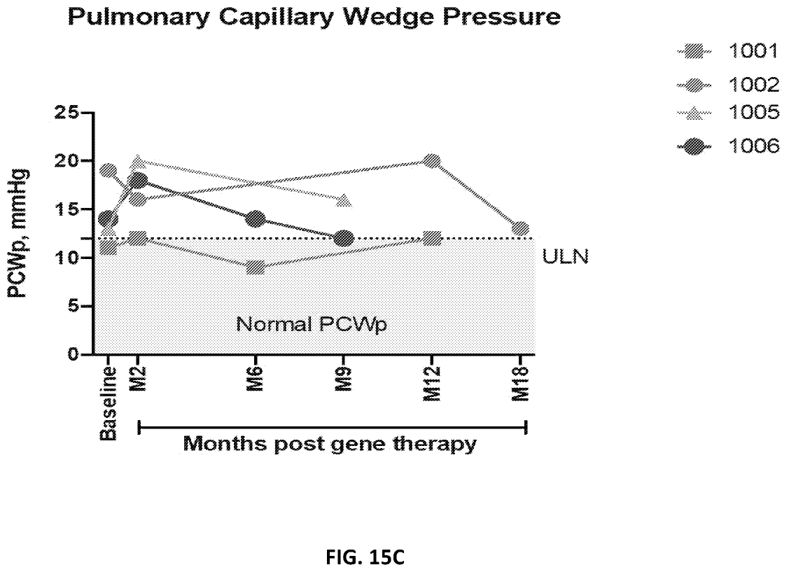

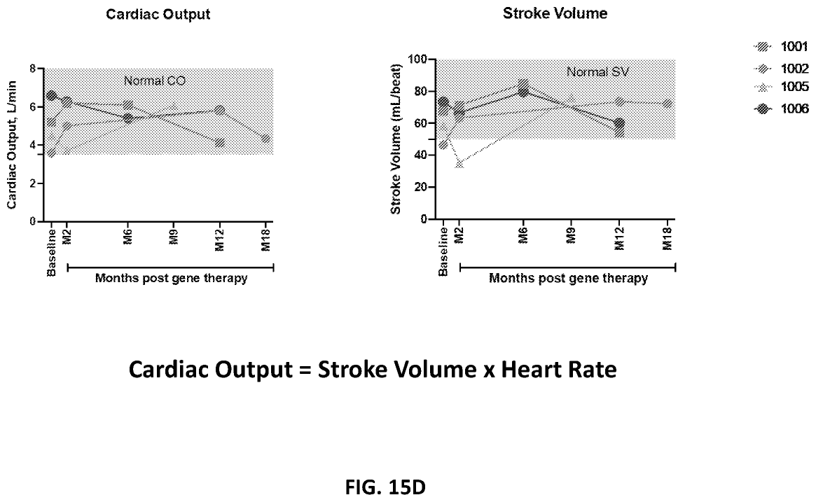

A hallmark of Danon cardiomyopathy in males especially is increased wall thickness resulting from hypertrophy as well as accompanying diastolic dysfunction. As illustrated in FIGS. 15A-15B, 4 of the 5 evaluable patients in the high and low dose either demonstrated stability or decrease in wall thickness as measured by serial echocardiography. In some subjects the decrease in wall thickness was accompanied by mild improvement or stability of their ejection fraction, which is a late manifestation of Danon disease (FIG. 15B). In addition to echo-based parameters, invasive hemodynamics enables measurement of pulmonary capillary wedge pressures which are a measure of diastolic dysfunction and left-sided filling pressures. Consistent with the other cardiac parameters the serial wedge pressures and the cardiac output/stroke volume in treated patients demonstrated either an improvement or stabilization (FIGS. 15C-15D). Given the natural history of the disease, this is in contrast to the normal progression of these patients.

Table 6 shows RP-A501 demonstrated stable cardiac vector copy numbers (VCN).

TABLE 6

| | | Cardiac VCN | |
|---|---|---|---|
| Cohort | Patient ID | Week 8 | Month 12 |
| Adult-Low Dose | 1001* | 0.5 | 0.6 |
| | 1002 | 6.5 | 1.5 |
| | 1005 | 2.5 | 1.9¹ |
| Adult-High Dose | 1006 | 3.9 | 1.1 |
| | 1007 | 5.9 | 6.8 (RV)² |
| | | | 9.2 (LV)² |

*Patient 1001 was only locally monitored for compliance for two weeks; longer compliance monitoring initiated after 100.
VCN = Vector Copies per diploid nucleus.
¹Month 9 data.
²Explanted heart samples at Month 5.

Table 7 shows endomyocardial LAMP2B protein expression by immunohistochemistry (IHC).

TABLE 7

| | | LAMP2B Protein Expression (by IHC)** | |
|---|---|---|---|
| Cohort | Patient ID | Week 8 | Month 12 |
| Adult-Low Dose | 1001* | 7.3% | 2.5% |
| | | | (Previously <15%)¹ |
| | 1002 | 36.9% | 67.8% |
| | 1005 | 17.6% | 92.4%² |
| Adult-High Dose | 1006 | 5.0% | 100% |
| | 1007 | 6.9% | 100%³ |

*Patient 1001 was only locally monitored for compliance for two weeks; longer compliance monitoring initiated after 1001.
**Endomyocardial biopsies stained for LAMP2 compared to normal control samples. Percent area of cell staining was quantitated using software in a blinded fashion from 2 to 14 sections. Qualitative assessment reported for samples with high variance.
¹Previously disclosed as a range due to high variance, now clarified.
²Month 9 data.
³Explant sample at Month 5

Table 8 shows endomyocardial LAMP2B Western Blot protein expression.

TABLE 8

| | | LAMP2B Protein Expression | |
|---|---|---|---|
| | | (by Western Blot) | |
| Cohort | Patient ID | Week 8 | Month 5-18 |
| Adult-Low Dose | 1001 | 20.7% | 17.9%¹ |
| | 1002 | 27.3% | 21.2%² |
| | 1005 | 42.8% | 61.1%³ |
| Adult-High Dose | 1006 | 14.6% | 18.2%¹ |
| | 1007 | 25.0% | RV: 45.1%⁴ |
| | | | LV: 44.0%⁴ |

¹Month 6 data; inadequate sample at Month 12.
²Month 18 data; inadequate sample at Month 12.
³Month 9 data.
⁵Explanted heart; Month 5 data.

As detailed above for the low and high dose cohorts from the Phase 1 study, relevant cardiac assessments, including serum heart failure markers, invasive hemodynamic output measurements, echocardiographic assessment and overall functional assessments, all demonstrate improvement in a disease in which even stabilization would represent a positive outcome compared to the natural history of progressive and fatal cardiomyopathy with median mortality of 19 years. These clinical evaluations are accompanied by evidence of LAMP2 expression in heart muscle and histologic improvement in the vacuolar pathology and myofibrillar disarray that are hallmarks of DD. For these reasons, the totality of the evidence—including both nonclinical and especially clinical data—overwhelmingly demonstrates the prospect of direct benefit of RP-A501.

RP-A501 was generally well tolerated at the low and high dose levels. All observed adverse effects were reversible with no lasting sequelae. Early transaminase and creatinine kinase elevations as well as platelet and hemoglobin decreases returned to baseline or eventually improved. RP-A501 r-AAV dose-dependent toxicity was seen in one of the two patients treated at the high dose level. The affected patient, who received the largest total dose, developed thrombotic microangiopathy (TMA) that fully resolved with supportive treatment including transient hemodialysis. Across both dose levels, the adverse events were reversible and largely resolved at 3 months following treatment with a tailored immune suppressive regimen.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method for treating Danon disease in a subject identified as suffering from or at risk for Danon disease and/or having an inactivating mutation in one or more isoforms of the LAMP-2 gene, comprising:
administering to the subject a therapeutically effective amount of a recombinant adeno-associated virus (rAAV) virion comprising a capsid and a vector genome,
wherein the vector genome comprises a polynucleotide sequence encoding a LAMP-2 protein, preferably a LAMP-2B protein.

2. The method of claim 1, wherein the therapeutically effective amount is less than about 2×10¹⁴ vector genomes (vg) per kilogram (kg) of the subject's body weight.

3. The method of claim 1, wherein the therapeutically effective amount is less than about 1.5×10¹⁴ vg/kg of the subject's body weight.

4. The method of claim 1, wherein the therapeutically effective amount is less than about 1×10¹⁴ vg/kg of the subject's body weight.

5. The method of any one of claims 1-4, wherein the therapeutically effective amount is at least about 1×10¹² vg/kg of the subject's body weight.

6. The method of any one of claims 1-4, wherein the therapeutically effective amount is at least about 1×10¹³ vg/kg of the subject's body weight.

7. The method of claim 1, wherein the therapeutically effective amount is about 6.7×10¹³ vg/kg of the subject's body weight.

8. The method of claim 1, wherein the therapeutically effective amount is about 1.1×10¹⁴ vg/kg of the subject's body weight.

9. The method of claim 1, wherein the therapeutically effective amount is about 2.0×10¹⁴ vg/kg of the subject's body weight.

10. The method of any one of claims 1-9, wherein the method further comprises administering to the subject an effective amount of tacrolimus.

11. The method of any one of claims 1-9, wherein the method further comprises administering to the subject an effective amount of rituximab.

12. The method of any one of claims 1-9, wherein the method comprises administering to the subject an effective amount of tacrolimus and administering to the subject an effective amount of rituximab.

13. The method of any one of claims 1-9, wherein the method comprises administering to the subject an effective amount of eculizumab.

14. The method of any one of claims 1-9, wherein the method further comprises administering to the subject an effective amount of rituximab; administering to the subject an effective amount of tacrolimus; and/or administering to the subject an effective amount of eculizumab.

15. The method of claim 13, wherein the subject is at risk for atypical hemolytic-uremic syndrome (aHUS), optionally aHUS resulting in reversible thrombocytopenia and/or acute kidney injury (AKI).

16. The method of any one of claims 1-15, wherein the method further comprises administering to the subject an effective amount of corticosteroids.

17. The method of claim 16, wherein the method comprises administering to the subject an effective amount of corticosteroids prior to administering the effective amount of tacrolimus.

18. The method of any one of claims 1-17, wherein the subject is a juvenile subject, optionally having an age of 8-14 years old and/or 15-17 years old.

19. The method of any one of claims 1-17, wherein the subject is a pediatric subject, optionally having an age of 0-8 years old.

20. The method of any one of claims 1-17, wherein the subject is an adult subject, optionally having an age of 18 years old or older.

21. The method of any one of claims 1-20, wherein the therapeutically effective amount of the AAV is administered intravenously.

22. The method of any one of claims 1-20, wherein the therapeutically effective amount of the AAV is administered by direct cardiac injection.

23. The method of any one of claims 1-20, wherein the therapeutically effective amount of the AAV is administered by intraperitoneal injection.

24. The method of any one of claims 1-23, wherein the method results in one or more of:
a) transduction of cardiomyocyte and/or skeletal muscle by the AAV;
b) expression of exogenous ribonucleic acid polynucleotide encoding LAMP-2B and/or expression of exogenous LAMP-2B protein, optionally in cardiomyocyte and/or skeletal muscle;
c) correction or improvement of one or more Danon disease-associated histologic abnormalities, optionally autophagic vacuoles or myofibrillar disarray;
d) correction or improvement of cardiomyocyte molecular marker expression; and/or
e) correction or improvement of cardiomyocyte histology.

25. The method of any one of claims 1-24, wherein the method results in one or more of:
a) sustained improvement or stabilization in cardiovascular pathophysiology, radiographic evaluation, and/or cardiopulmonary exercise/physiologic parameters;
b) correction of LAMP2B gene and/or protein expression;
c) improvement in Danon disease-associated histologic abnormalities; and/or
d) tolerable immunologic response to the AAV.

26. The method of any one of claims 1-25, wherein the method results in one or more of:
a) sustained improvement or stabilization in cardiovascular pathophysiology, radiographic evaluation, and/or cardiopulmonary exercise/physiologic parameters;
b) correction of LAMP2B gene and/or protein expression;
c) improvement in Danon disease-associated histologic abnormalities; and/or
d) tolerable immunologic response to the AAV.

27. The method of any one of claims 1-26, wherein the method results in one or more of:
a) low incidence and/or intensity of treatment-emergent adverse events (TEAEs) and SAEs;
b) low incidence of cardiac interventions, including cardiac transplant, implantable cardioverter-defibrillator, or pacemaker placement, electrophysiologic ablative procedure for cardiac conduction aberrancy, or subsequent hospitalizations for heart failure;
c) low immune response to the AAV, optionally antibodies or T-lymphocytes reactive to AAV and/or to LAMP-2B protein;
d) low incidence and/or intensity of hepatotoxicity, optionally low changes in liver transaminases (AST and ALT), GGT, bilirubin, and ALP; and/or
e) low incidence and/or intensity of h coagulopathy, based on changes from baseline in platelet count, prothrombin time (PT, or International Normalized Ratio (INR)), activated partial thromboplastin time (aPTT), fibrinogen, D-dimer, thrombin-antithrombin complex (TAT), and complement components (complement 3 (C3), complement 4 (C4), and serum membrane attack complex (sC5b-9).

28. The method of any one of claims 1-27, wherein the AAV comprises an expression cassette comprising the polynucleotide sequence encoding the LAMP-2B protein operatively linked to a promoter, and wherein the polynucleotide sequence shares at least 95% identity to SEQ ID NO: 2 and/or the LAMP-2B protein shares at least 95% identity to SEQ ID NO: 1.

29. The method of any one of claims 1-28, wherein the polynucleotide sequence comprises or consists of SEQ ID NO: 2 and/or the LAMP-2B protein comprises or consists of SEQ ID NO: 1.

30. The method of any one of claims 1-29, wherein the promoter is a CAG promoter.

31. The method of claim 30, wherein the promoter comprises an enhancer/promoter region that shares at least 95% identity to SEQ ID NO: 22.

32. The method of claim 30, wherein the enhancer/promoter region comprises or consists of SEQ ID NO: 22.

33. The method of claim 28, wherein the expression cassette comprises, in 5′ to 3′ order:
(a) an enhancer/promoter region that comprises SEQ ID NO: 22;
(b) the polynucleotide sequence that encodes the LAMP-2B protein, wherein the polynucleotide sequence comprises SEQ ID NO: 3;
(c) a 3′ UTR sequence comprising SEQ ID NO: 27; and/or
(d) a poly-adenylation sequence comprising SEQ ID NO: 7.

34. The method of claim 28, wherein the expression cassette is flanked by: (i) a 5′ ITR that comprises SEQ ID NO: 11; and (ii) a 3′ ITR that comprises SEQ ID NO: 12.

35. The method of claim 28, wherein the expression cassette comprises SEQ ID NO: 8.

36. The method of claim 28, wherein the capsid is an AAV9 capsid.

37. The method of claim 36, wherein the AAV9 capsid comprises one or more capsid proteins that comprise amino acids 1 to 736 of SEQ ID NO: 28, amino acids 138 to 736 of SEQ ID NO: 28, or amino acids 203 to 736 of SEQ ID NO: 28.

38. A unit dose, pharmaceutical composition, or composition for use in treating Danon disease, comprising a therapeutically effective amount of an adeno-associated virus (AAV) comprising a polynucleotide sequence encoding a LAMP-2 protein, preferably a LAMP-2B protein.

39. The unit dose, pharmaceutical composition, or composition for use of claim 38, wherein the therapeutically effective amount is less than about 2×10¹⁴ vector genomes (vg) per kilogram (kg) of the subject's body weight.

40. The unit dose, pharmaceutical composition, or composition for use of claim 38, wherein the therapeutically effective amount is less than about 1.5×10¹⁴ vg/kg.

41. The unit dose, pharmaceutical composition, or composition for use of claim 38, wherein the therapeutically effective amount is less than about 1×10¹⁴ vg/kg.

42. The unit dose, pharmaceutical composition, or composition for use of any one of claims 38-41, wherein the therapeutically effective amount is at least about 1×10¹² vg/kg.

43. The unit dose, pharmaceutical composition, or composition for use of any one of claims 38-41, wherein the therapeutically effective amount is at least about 1×10¹³ vg/kg.

44. The unit dose, pharmaceutical composition, or composition for use of any one of claims 38-41, wherein the therapeutically effective amount is about 6.7×10¹³ vg/kg.

45. The unit dose, pharmaceutical composition, or composition for use of any one of claims 38-41, wherein the therapeutically effective amount is about 1.1×10¹⁴ vg/kg.

46. The unit dose, pharmaceutical composition, or composition for use of any one of claims 38-41, wherein the therapeutically effective amount is about 2.0×10¹⁴ vg/kg.

47. The unit dose, pharmaceutical composition, or composition for use of any one of claims 38-46, wherein the AAV comprises an expression cassette comprising the polynucleotide sequence encoding the LAMP-2B protein operatively linked to a promoter, and wherein the polynucleotide sequence shares at least 95% identity to SEQ ID NO: 2 and/or the LAMP-2B protein shares at least 95% identity to SEQ ID NO: 1.

48. The unit dose, pharmaceutical composition, or composition for use of any one of claims 38-47, wherein the polynucleotide sequence comprises or consists of SEQ ID NO: 2 and/or the LAMP-2B protein comprises or consists of SEQ ID NO: 1.

49. The unit dose, pharmaceutical composition, or composition for use of any one of claims 38-48, wherein the promoter is a CAG promoter.

50. The unit dose, pharmaceutical composition, or composition for use of any one of claims 38-49, wherein the promoter comprises an enhancer/promoter region that shares at least 95% identity to SEQ ID NO: 22.

51. The unit dose, pharmaceutical composition, or composition for use of any one of claims 38-50, wherein the enhancer/promoter region comprises or consists of SEQ ID NO: 22.

52. The unit dose, pharmaceutical composition, or composition for use of any one of claims 38-51, wherein the expression cassette comprises, in 5′ to 3′ order:
(a) an enhancer/promoter region that comprises SEQ ID NO: 22;
(b) the polynucleotide sequence that encodes the LAMP-2B protein, wherein the polynucleotide sequence comprises SEQ ID NO: 3;
(c) a 3′ UTR sequence comprising SEQ ID NO: 27; and/or
(d) a poly-adenylation sequence comprising SEQ ID NO: 7.

53. The unit dose, pharmaceutical composition, or composition for use of any one of claims 38-52, wherein the expression cassette is flanked by: (i) a 5′ ITR that comprises SEQ ID NO: 11; and (ii) a 3′ ITR that comprises SEQ ID NO: 12.

54. The unit dose, pharmaceutical composition, or composition for use of any one of claims 38-53, wherein the expression cassette comprises SEQ ID NO: 8.

55. The unit dose, pharmaceutical composition, or composition for use of any one of claims 38-54, wherein the capsid is an AAV9 capsid.

56. The unit dose, pharmaceutical composition, or composition for use of claim 55, wherein the AAV9 capsid comprises one or more capsid proteins that comprise amino acids 1 to 736 of SEQ ID NO: 28, amino acids 138 to 736 of SEQ ID NO: 28, or amino acids 203 to 736 of SEQ ID NO: 28.

57. A kit comprising the unit dose, pharmaceutical composition, or composition for use of any one of claims 38-55; and instructions for use in treating Danon disease.

58. The kit of claim 57, wherein the kit further comprises one or more unit dose, pharmaceutical composition, or composition comprising one or more of: rituximab; tacrolimus; eculizumab; and a corticosteroid.
